(19) United States
(12) Patent Application Publication
(10) Pub. No.: US 2003/0082722 A1
(43) Pub. Date: May 1, 2003

(54) Method for amplifying expression from a cell specific promoter

(76) Inventor: Bingliang Fang, Pearland, TX (US)

Correspondence Address: Steven L. Highlander, FULBRIGHT & JAWORSKI L.L.P., Suite 2400, 600 Congress Avenue, Austin, TX, 78701-3171, US

(21) Appl. No.: 10/212,667

(22) Filed: Aug. 5, 2002

Related U.S. Application Data
(60) Provisional application No. 60/310,905, filed on Aug. 8, 2001.

(51) Int. Cl. C12P 21/02; C12N 5/06; C12N 15/00

(52) U.S. Cl. 435/69.1; 435/455; 435/325; 435/320.1

(57) ABSTRACT

The present invention provides, in one aspect, methods for selective expressing gene products using a binary or bicistronic expression system based on the use of a tissue-preferential promoter to drive expression of a transcriptional activator, which in turn drives a gene of interest. In another aspect, the invention provides for methods of cancer therapy comprising expressing Bax, TRAIL or various other therapeutic proteins using a tissue preferential promoter such as hTERT or CEA, optionally coupled with a binary or a bicistronic expression system.

The present application claims priority to co-pending U.S. Provisional Patent Application Serial No. 60/310,905 filed on Aug. 8, 2001. The entire text of the above-referenced disclosure is specifically incorporated herein by reference without disclaimer.

The government owns rights in the present invention pursuant to grant number P50CA70907-01 from the National Institutes of Health (University of Texas SPORE in Lung Cancer).

### BACKGROUND OF THE INVENTION

1. Field of the Invention

The present invention relates generally to the fields of oncology, molecular biology and gene therapy. More particularly, it concerns gene therapy of diseases where limiting the expression of therapeutic genes to certain cells and tissues is required for treatment benefit, for example, in reducing toxicity and/or enhancing effects of the delivered genes.

2. Description of Related Art

Targeting of pharmaceutical effects of a therapeutic gene to a specific site or tissue is a highly desirable goal in cancer gene therapy. One of the common approaches to targeted expression is to control the gene expression via tissue- or cell-specific promoters. Several promoters already identified are more active in particular tumor types than in the tissues or organs from which they arise and so have been extensively exploited to restrict transgene expression in tumors after non-specific gene delivery. For example, the tyrosinase promoter has been used to achieve specific expression of therapeutic genes in melanoma; the carcinoembryonic antigen promoter, in colorectal and lung cancer cells; the MUC1 promoter, in breast cancer; and the E2F promoter in cancers with a defective retinoblastoma gene.

While these reports suggest that achieving relatively tumor-specific transgene expression is feasible, they also revealed several limitations. A major problem of most promoters currently used to drive tumor-specific expression is their weak transcriptional activity. In fact, most are much weaker than commonly used viral promoters such as the cytomegalovirus early promoter, Rous sarcoma virus long terminal repeats and the SV40 promoter. The consequence is low gene expression, with a corresponding lack of antitumor activity for the expressed therapeutic gene. Thus, while tumor specific promoters provide an interesting option for cancer therapy, their inherent weakens—low level transcription—presents a fundamental impediment in their use.

### SUMMARY OF THE INVENTION

Thus, in accordance with the present invention, there is provided a method for expressing gene product in a cell type-preferential manner comprising (a) providing a first expression cassette comprising a cell type-preferential promoter that directs the expression of a nucleic acid encoding a transcription factor; (b) providing a second expression cassette comprising an inducible promoter, responsive to the transcription factor, that directs the expression of a nucleic acid encoding a selected polypeptide; and (c) transferring the first and second expression cassettes into a cell in which the cell type-specific preferential promoter is active, wherein the transcription factor is expressed and directs expression of the selected polypeptide.

The cell type-preferential promoter may be hTERT, CEA, PSA, probasin, ARR2PB, or AFP. The transcription factor may be GAL4-VP16 fusion and the inducible promoter may be GAL4/TATA. Alternatively, the transcription factor may be tetR-VP 16 fusion and the inducible promoter may be the tet operator. The first and second expression cassettes may be located in different expression constructs or located in the same expression construct. The expression cassettes may be located in a viral expression construct or a non-viral expression construct. The viral expression construct may be adenoviral expression construct, a herpesviral expression construct, a retroviral expression construct, a vaccinia viral expression construct, an adeno-associated viral expression construct or a polyoma viral expression construct. The cell may be a tumor cell, such as a brain tumor cell, a head & neck tumor cell, an esophageal tumor cell, a lung tumor cell, a thyroid tumor cell, a stomach tumor cell, a colon tumor cell, a liver tumor cell, a kidney tumor cell, a prostate tumor cell, a breast tumor cell, a cervical tumor cell, an ovarian tumor cell, a testicular tumor cell, a rectal tumor cell, a skin tumor cell or a blood tumor cell.

The selected polypeptide may be a tumor suppressor, an inducer of apoptosis, a cytokine, an enzyme or a toxin. The tumor suppressor may be, for example, p53, Rb, PTEN, BRCA1 or BRCA2. The inducer of apoptosis may be, for example, Bax, Bad, Bid, Bik, Bak, TRAIL, FasL, Noxa, PUMA, p53AIP1, TGF-β, Granzyme A or Granzyme B. The cytokine may be, for example, IL-2, IL-4, IL-10, IL-12, GM-CSF, MCP-3, TNF-α or INF-β. The enzyme may be, for example, cytosine deaminase. The toxin may be, for example, ricin A chain, cholera toxin and pertussis toxin.

In another embodiment, there is provided a method of treating a human subject having cancer comprising (a) providing a first expression cassette comprising a CEA or hTERT promoter that directs the expression of a nucleic acid encoding a transcription factor; (b) providing a second expression cassette comprising an inducible promoter, responsive to the transcription factor, that directs the expression of a nucleic acid encoding a therapeutic polypeptide; and (c) transferring the first and second expression constructs into a cancer cell in the subject, wherein the transcription factor is expressed and directs expression of the therapeutic polypeptide.

The cell type-preferential promoter may be hTERT, CEA, PSA, probasin, ARR2PB, or AFP. The transcription factor may be GAL4-VP16 fusion and the inducible promoter may be GAL4/TATA. Alternatively, the transcription factor may be tetR-VP16 fusion and the inducible promoter may be the tet operator. The first and second expression cassettes may be located in different expression constructs or located in the same expression construct. The expression cassettes may be located in a viral expression construct or a non-viral expression construct. The viral expression construct may be adenoviral expression construct, a herpesviral expression construct, a retroviral expression construct, a vaccinia viral expression construct, an adeno-associated viral expression construct or a polyoma viral expression construct. The cell may be a tumor cell, such as a brain tumor cell, a head & neck tumor cell, an esophageal tumor cell, a lung tumor cell, a thyroid tumor cell, a stomach tumor cell, a colon tumor cell, a liver tumor cell, a kidney tumor cell, a prostate tumor cell, a breast tumor cell, a cervical tumor cell, an ovarian tumor cell, a testicular tumor cell, a rectal tumor cell, a skin tumor cell or a blood tumor cell.

The selected polypeptide may be is a tumor suppressor, an inducer of apoptosis, a cytokine, an enzyme or a toxin. The tumor suppressor may be, for example, p53, Rb, PTEN, BRCA1 or BRCA2. The inducer of apoptosis may be, for example, Bax, Bad, Bid, Bik, Bak, TRAIL, FasL, Noxa, PUMA, p53AIP1, TGF-β, Granzyme A or Granzyme B. The cytokine may be, for example, IL-2, IL-4, IL-10, IL-12, GM-CSF, MCP-3, TNF-α or INF-β. The enzyme may be, for example, cytosine deaminase. The toxin may be, for example, ricin A chain, cholera toxin and pertussis toxin. The method may further comprise administering a second cancer therapy comprising surgery, immunotherapy, chemotherapy or radiation therapy.

In yet another embodiment, there is provided a method for treating a human cancer patient comprising (a) providing a non-viral expression cassette comprising an hTERT promoter that directs the expression of a nucleic acid encoding a tumor suppressor or an inducer of apoptosis; and (b) administering the expression cassette into the subject, wherein the tumor suppressor or inducer of apoptosis is expressed and inhibits growth of cancer cells, thereby treating the cancer. The cancer cells may simply be inhibited in their growth, or they may be killed. The method may further comprise administering a second cancer therapy comprising surgery, immunotherapy, chemotherapy or radiation therapy. The nucleic acid encoding a tumor suppressor or an inducer of apoptosis may further encode a screenable marker fused to the tumor suppressor or the inducer of apoptosis.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Basal and augmented transgene expression from the CEA promoter in cultured cells. A549 cells and NHFB were treated with adenoviral vectors. β-Galactosidase activities were determined at 48 h after treatment and expressed as relative light units (RLU)/μg of cellular protein. Each value represents the mean+S.D. of three assays. The differences between expression induced by Ad/CEA-LacZ and Ad/GT-LacZ +Ad/CEA-GV16 in each cell line are indicated here. In both cell lines, the difference in expression is significant (P<0.01).

FIG. 2. Basal and augmented transgene expression from the CEA promoter in subcutaneous tumors. Subcutaneous tumors derived from A549 cells were established in nude mice and treated with various adenoviral vectors. β-galactosidase activities determined by enzymatic assay. The treatment is indicated under each bar. β-galactosidase activities were expressed as RLU/μg of cellular protein. Each value represents the mean±S.D. for at least five animals.

FIG. 3. Cell viability after vector treatment. Cell viability was determined in three cell lines (A549, LoVo, and NHFB) by XTT assay at 0, 24, 48, and 72 h after adenovirus vector treatment. Cells treated with PBS were used as a control, and their viability was set at 1. Each value is the mean±S.D. for two quadruplicate assays. By 48 h, treatment with Ad/GT-Bax+Ad/PGK-GV16 had significantly reduced cell viability in all three lines when compared with controls at 48 h (P<0.01), whereas treatment with Ad/GT-Bax+Ad/CEA-GV16 had significantly reduced viability only in A540 and LoVo cells (P<0.01).

FIG. 4. Suppression of tumor growth by adenovirus-mediated gene transfer. Subcutaneous tumors derived from LoVo cells were treated with various vectors. Tumor volume was monitored over time after inoculation of tumor cells. Arrow, time point where treatment was given. Values represent the mean±SD of at least five animals per group. Treatment with Ad/CEA-GV16+Ad/GT-Bax or Ad/PGK-GV16+Ad/GT-Bax differs significantly from other control groups (P≦0.01).

FIG. 5. In vitro analysis of hTERT promoter activities. Biochemical analysis of β-galactosidase activities. The enzyme activity is presented as relative light units (RLU)/μg protein. Values are mean+SD for three assays.

FIG. 6. In vivo assessment of hTERT promoter. BALB/c mice treated with various vectors and analyzed for β-galactosidase activity. Biochemical analysis of β-galactosidase. β-galactosidase activities are presented as relative light units (RLU)/μg protein. Values are means+SD for five mice per group.

FIGS. 7A-7B. In vitro assessment of the antitumor effect of the Bax gene induced by hTERT or PGK promoter. FIG. 7A. Flow cytometric analysis of apoptotic (sub-G1) cells. Cell lines are indicated to the left of panels, treatments at the top of panels, and apoptotic cell percentages underneath each panel. FIG. 7B. Cell viability was determined by XTT assay after treatments. Cells treated with PBS were used as a control, and their viability was set at 100%. Values are means±SD for two quadruplicate assays. ♦=PBS, ▪=Ad/CMV-GFP+Ad/PGK-GV16, ▴=Ad/GT-Bax +Ad/CMV-GFP, ◯=Ad/GT-Bax+Ad/hTERT-GV16, [symbol] =Ad/GT-Bax+Ad/PGK-GV16.

FIG. 8. Suppression of tumor growth by adenovirus-mediated gene transfer. Subcutaneous tumors derived from H1299 cells were treated with various vectors as shown above. Tumor volume was monitored over time (days) after inoculation of tumor cells. Values represent the mean±SD of at least eight mice per group. Arrow indicates the time point where treatment (9×10¹⁰ total viral particles/mouse/treatment) was given. ♦=PBS, ▪=Ad/E1⁻, ▴=Ad/GT-LacZ+Ad/hTERT-GV16, □=Ad/GT-Bax+Ad/hTERT-GV16, ◯=Ad/GT-Bax+Ad/PGK-GV16.

FIG. 9. In vivo liver toxicity of Bax gene induced by the hTERT or PGK promoter. Serum levels of AST and ALT 48 h after intravenous viral infusion. Values represent the means of three animals per group; bars, SD.

FIG. 10. Diagram of Ad/gTRAIL. The E1 region (map unit 1.3˜9.3) of human adenovirus type 5 is replaced by therapeutic sequences composed of expression cassettes for the GAL/VP16 and GFP/TRAIL genes. Polyadenylation signal sequences from BGH and SV40 genes are used for these cassettes.

FIGS. 11A-11B. Transgene expression and cell killing effects of Ad/gTRAIL in vitro. FIG. 11A. Flow cytometric assay. H460, A549, DLD1, and Lovo cells were harvested 48 h after treatment, as indicated above each column. Levels of GFP expression (upper panel) and apoptotic cell death (lower panel) were determined by FACS. Percentage of GFP+ or apoptotic “sub-G1” cells is shown. FIG. 11B. Cell viability as determined by XTT assay. Cells were treated with PBS (●), Ad/CMV-GFP (▪), Ad/gTRAIL (▴), and Ad/GT-TRAIL+Ad/PGK-GV16 (*). Viability is expressed relative to that of cells treated with PBS, which was set at 1. Values represent the means ±s.d. of quadruplicate wells. In all the four cell lines, viability after treatment with TRAIL-expressing vectors versus control vectors differed significantly (p<0.001) at 2, 4, and 7 d after treatment.

FIG. 12. Suppression of tumor growth by Ad/gTRAIL in vivo. Tumor growth in subcutaneous xenograft model derived from DLD1 cells. Tumor volume was monitored over time (days) after inoculation of tumor cells. The values represent mean±standard error of 10 animals/group. The growth curve for tumors treated with Ad/gTRAIL or Ad/GT-TRAIL+Ad/PGK-GV16 overlaps almost completely. Both differed significantly from treatment with PBS or vector controls (p<0.001). Arrow, time point at which treatment was given..

FIGS. 13A-13B. Effects of Ad/gTRAIL on NHPH and NHFB. FIG. 13A. Flow cytometric assay. The analysis was performed as described in FIGS. 11A and 11B. Upper panel, levels of GFP expression. Lower panel, apoptotic cell death. Percentage of GFP-positive or apoptotic “sub-G1” cells is shown. FIG. 13B. Cell viability assay. NHPHs and NHFBs were treated with PBS (●), Ad/CMV-GFP (▪), Ad/gTRAIL (▴), and Ad/GT-TRAIL+Ad/PGK-GV16 (*). Viability was expressed relative to that of cells treated with PBS, which is set at 1. Values represent the means±s.d. of quadruplicate wells. Cell viability was significantly reduced in NHPHs treated with Ad/GT-TRAIL+Ad/PGK-GV16 when compared with that of other groups (p<0.001) at 2, 4, and 7 days after treatment.

FIG. 14. In vivo assessment after systemic delivery in Balb/c mice. Activity of serum liver enzymes, AST and ALT, at day 14. Open, PBS; dotted, Ad/CMV-GFP; striped, Ad/gTRAIL; and black, Ad/GT-TRAIL+Ad/PGK-GV16.

FIGS. 15A-15B. Transgene expression and cell-killing effects of Ad/gTRAIL in vitro. FIG. 15A. Transgene expression and apoptosis induction. FIG. 15B. Cell viability determined by XTT assay.

FIG. 16. Dose-response curve for TRAIL protein.

FIGS. 17A-17B. Apoptdsis of 231/ADR induced by Ad/gTRAIL. FIG. 17A. Dose response curves of MDA-MB-231 and 231/ADR cells incubated with different concentrations of doxorubicin for 48 h. FIG. 17B. Cell killing effect of Ad/gTRAIL in 231/ADR cells.

FIGS. 18A-18B. Transgene expression and cell killing in normal and transformed breast cells. FIG. 18A. The percentage of sub-G1 cells and GFP levels in MCF10A, MCF10F, NPMEC, and NMEC cells treated with Ad/gTRAIL were determined by flow cytometry 48 h after treatment. FIG. 18B. Cell killing effect of Ad/gTRAIL was tested by the XTT assay in MCF10A, MCF10F, NPMEC, and NMEC cells.

FIGS. 19A-19D. Antitumor effect of Ad/gTRAIL in vivo. Tumor growth (FIG. 19A, FIG. 19C) and survival (FIG. 19B, FIG. 19D) in animals bearing subcutaneous xenografts derived from MDA-MB-231 (FIG. 19A, FIG. 19B) or 231/ADR (FIG. 19C, FIG. 19D) cells.

FIG. 20. In vitro analysis of hTERT promoter activities. β-galactosidase activities are presented as relative light units (RLU)/μg protein. Values are means±s.d. for three assays.

FIGS. 21A-21B. In vitro assessment of the antitumor effect of the Bax gene on tumor cells induced by the hTERT or PGK promoter. FIG. 21A. Cell viability determined by XTT assay. Cells treated with PBS were used as a control, and their viability was set at 100%. Values are means±s.d. for two quadruplicate assays. (♦), PBS; (▪), Ad/CMV-GFP+Ad/PGK-GV16; (▴), Ad/GT-Bax+Ad/CMV-GFP; (□), Ad/GT-Bax+Ad/PGK-GV16; (O), Ad/GT-Bax+Ad/hTERT-GV16. FIG. 21B. Flow cytometric analysis of apoptotic (sub-G1) cells on UV2237m cells. Treatments are at the top of panels, and apoptotic cell percentages underneath each panel.

FIG. 22. Suppression of syngenic tumor growth by hTERT-induced and tumor-specific Bax gene expression. Subcutaneous tumors derived from UV-223m cells were treated with various vectors. Tumor volumes were moniored over time (days) after inoculation of tumor cells. values represent the mean±s.d. of ten mice per group. Arrow indicates the time point where treatment (9×10¹⁰ total viral particles/mouse/treatment) was given. (♦), PBS; (▪), Ad/E1-; (▴), Ad/GT-LacZ+Ad/hTERT-GV16; (O), Ad/GT-Bax+Ad/hTERT-GV16; (□), Ad/GT-Bax+Ad/PGK-GV16. Note that the results of treatment with Ad/GT-Bax+Ad/hTERT-GV16 or Ad/GT-Bax+Ad/PGK-GV16 differ significantly from those of the other control groups by ANOVA (P≦0.01).

FIG. 23. Analysis of hTERT promoter activities in human bone marrow CD34⁺ progenitor cells.

FIGS. 24A-24B. Characterization of DLD1/Bax-R and DLD1/TRAIL-R cells. FIG. 24A. Parental DLD1. DLD1/Bax-R and DLD1/TRAIL-R DLD1 cells infected with different adenovirus at a total MOI of 1000 vp/cells. FIG. 24B. Cell viability was determined 24, 48 and 72 h after treatment. Cells treated with PBS were used as a mock control, and their viability was set as 100%. Values are means±s.d. for quadruplicate assays. In parental DLD1 cells, levels of apoptosis after treatment with Ad/hTERT-GV16+Ad/GT-Bax or Ad/gTRAIL differed significantly from levels after treatments with PBS or Ad/CMV-GFP (P≦0.01), whereas there were no differences in apoptosis levels among treatment groups for DLD1/Bax-R or DLD1/TRAIL-R cells. (⋄), PBS; (□), Ad/CMV-GFP; (Δ), Ad/gTRAIL; (O), Ad/hTERT-GV16+Ad/GT-Bax.

FIGS. 25A-25B. Cell killing by dose escalation. Parental DLD1 cells were infected with Ad/hTERT-GV16+Ad/GT-Bax at a total MOI of 1000 vp/cell. DLD1/Bax-R were infected with Ad/hTERT-GV16+Ad/GT-Bax at a total MOI of 10,000 vp/cell. Cells treated with PBS were used as a mock control. Left, cell lines; top, treatments; number within each panel, percentages of apoptotic cells. FIG. 25A. Percentages of apoptotic (sub-G1) cells determined by FACS 48 h after treatment. FIG. 25B. Cell viability determined 24, 48 and 72 h after treatment. (⋄), PBS; (□), Ad/CMV-GFP; (O), Ad/hTERT-GV16+Ad/GT-Bax.

FIGS. 26A-26B. Effects of adenoviral vectors expressing alternative proapoptotic genes. Parental DLD1, DLD1/Bax-R and DLD1/TRAIL-R cells were infected with different adenoviruses at a total MOI of 1000 vp/cell. Cells treated with PBS were used as a negative control. FIG. 26A. Percentages of apoptotic (sub-G1) cells were determined by FACS 48 h after treatment. Left, cell lines; top, treatments; number within each panel, percentages of apoptotic cells. FIG. 26B. Cell viability was determined 24, 48, and 72 h after treatment. Cells treated with PBS were used as a positive control, and their viability was set as 100%. Values are means±s.d. for quadruplicate assays. In DLD1 cells, apoptosis levels after treatments with Ad/hTERT-GV16+Ad/GT-Bax or Ad/hTERT-GV16+Ad/GT-Bak or Ad/gTRAIL differed significantly from levels after treatments with PBS or Ad/CMV-GFP (P≦0.01). In DLD1/Bax-R cells, apoptosis levels after treatment with Ad/gTRAIL differed significantly from levels after treatment with PBS or Ad/CMV-GFP (P≦0.01), Ad/hTERT-GV16+Ad/GT-Bax, and Ad/hTERT-GV16+Ad/GT-Bak (P≦0.05). In DLD1/TRAIL-R cells, apoptosis levels after treatment with Ad/hTERT-GV16+Ad/GT-Bax or Ad/hTERT-GV16+Ad/GT-Bak differed significantly from levels after treatment with PBS, Ad/CMV-GFP, or Ad/gTRAIL (P≦0.01). (⋄), PBS; (□), Ad/CMV-GFP; (Δ), Ad/gTRAIL; (O), Ad/hTERT-GV16+Ad/GT-Bax; (−), Ad/hTERT-GV16+Ad/GT-Bak.

FIG. 27. Effect of Bcl-xL over-expression. Percentages of apoptotic cells as determined by FACS. Bcl-xL-transfected DLD1 clones 1-7 were transfected with different adenoviruses, each at a total MOI of 1000 vp/cell. Cell treated with PBS were used as mock control. Percentages of apoptotic (sub-G1) cells were determined by FACS 48 h after treatment. Left, Bcl-xL-transfected DLD1 clones; top; treatments; number within each panel, percentages of apoptotic cells.

FIG. 28. Schematic diagram of Ad/hTERT-gBax (Ad/gBax).

FIGS. 29A-29B. In vitro assessment of the antitumor effect of the GFP±Bax gene on tumor cells induced by Ad/hTERT±gBax. FIGS. 29A. Cell viability determined by XTT assay. Cells treated with PBS were used as a control, and their viability was set at 100%. Values are means+s.d. for two quadruplicate assays. (▴), PBS; (▪), Ad/CMV-GFP; (□), Ad/hTERT-gBax; (O), Ad/GT-Bax+Ad/hTERT-GV16. Dashed line in NHFB: Ad/GT±Bax+Ad/PGK±GV16. FIG. 29B. Flow cytometric analysis of apoptotic (sub-G1) cells in cancer and normal cell. FACS analysis was performed 72 h after virus treatment.

FIG. 30. Suppression of tumor growth by hTERT promote-induced and tumor-specific Bax gene expression. Subcutaneous tumors derived from H1299 cells were treated with various vectors: Tumor volumes were monitored over time (days) after inoculation of the tumor cells. Values are the mean±s.d with 10 mice per group. The arrow indicates the time point where treatment (9×10¹⁰ total viral particles/mouse/treatment) was given. (▴), PBS; (▪), Ad/CMV-GFP; (□), Ad/gBax, (O), Ad/GT-Bax+Ad/hTERT-GV16. Note that the results of treatment with Ad/gBax and Ad/GT±Bax+Ad/hTERT±GV16 differ significantly from those of the other control groups as shown by ANOVA (P≦0.01).

FIGS. 31A-31B. Transgene expression and apoptosis induction in cancer cells. FIG. 31A. Diagram of induction of GFP/TRAIL and Bax. Ad/gTRAIL contains expression cassettes for the GAL4/VP16 (GV16) and GFP/TRAIL genes in replace of the E1 region (map unit 1.3˜9.3) of human adenovirus type 5. FIG. 31B. H1299, DOV13 and SKOV3ip cells were treated using various vectors. Expression of GFP and GFP/TRAIL were determined by FACS analysis 48 h after treatment. Left, cell lines; top, treatments; number within each panel, percentage of GFP-positive cells.

FIGS. 32A-32B. Apoptosis induction and cell-killing effects in vitro. FIG. 32A. H1299, DOV13 and SKOV3ip cells treated using various vectors were tested for apoptosis induction by analyzing the cellular DNA content using a FACS. Left, cell lines; top, treatments; number within each panel, percentage of apoptotic cells. FIG. 32B. Cell viability was determined within 1 week after treatments. Cells treated using PBS were used as mock controls, and their viability was set as 1.0. Each value is the means±s.d. for quadruplicate assays. (♦), PBS; (□), Ad/gTRAIL; (▴), Ad/gTRAIL plus Ad/GT-Bax; (▪), Ad/hTERT-GV16 plus Ad/GT-Bax, and (*), Ad/CMVGFP plus Ad/GT-Bax.

FIGS. 33A-33B. Transgene expression and apoptosis induction in normal human ovarian surface epithelial cells (NHOE). FIG. 33A. GFP or GFP/TRAIL expression (upper panel) and apoptosis (low panel) 48 h after treatment as indicated above each panel. Number within each panel, percentage of GFP-positive cells (upper panel) and apoptotic sub-G1 cells (low panel). FIG. 33B. Cell viability was determined within 1 week after treatments. Cells treated using PBS were used as mock control, and their viability was set as 1.0. Values are mean±s.d. for quadruplicate assays. (♦), PBS; (□), Ad/gTRAIL; (▴), Ad/gTRAIL plus Ad/GT-Bax; (▪), Ad/hTERT-GV16 plus Ad/GT-Bax, and (*), Ad/CMV-GFP plus Ad/GT-Bax; (⋄), Ad/PGK-GV16 plus Ad/GT-Bax. Only the treatment using Ad/PGK-GV16 plus Ad/GT-Bax elicited significant cell killing in normal cells.

FIGS. 34A-34B. In vivo antitumor activity. FIG. 34A. Volumes of the largest tumor in the peritoneal cavity; ascites volumes; body weight. The volumes of the largest tumors and ascites were determined 28 days after tumor-cell inoculation, while body weight was measured both 4 and 28 days after tumor-cell inoculation. Treatment was started 4 days after tumor-cell inoculation. Treatment using the GFP/TRAIL- and Bax-expressing vectors both separately and combined resulted in a significant difference in the volumes of the largest tumors, volumes of ascites and body weight when compared with treatment using PBS or a control vector (P≦0.05). FIG. 34B. Survival curves for animals bearing abdominally spread SKOV3 tumors. The animals received four treatments using PBS, Ad/gTRAIL, Ad/GT-Bax, Ad/gTRAIL plus Ad/GT-Bax, or Ad/hTERT-LacZ. Survival was then monitored: the survival durations in animals receiving treatment using TRAIL, Bax or TRAIL plus Bax were significantly different from those in the mice that received treatment using controls (P≦0.05). In addition, the survival duration in animals that received treatment using TRAIL plus Bax were significantly different from those in animals that received treatment using Bax or TRAIL alone (P≦0.05).

FIG. 35. Serum AST and ALT levels after intraperitoneal administration of hTERT-LacZ. Serum samples were collected before treatment started (day 0), and one (day 1) and 14 days (day 14) after the last treatment.

FIG. 36. Sensitizing TRAIL resistant colon cancer cells DLD1/TRAIL-R, to Ad/gTRAIL by doxorubicin (ADR); floxuridine (FuDR); fluorouracil (5-FU) and mutamyci (MMC).

FIG. 37. Ad/CMV-LacZ delivered by aerosol. Biochemical analysis of cells treated with protamine, hydrocortisone or the combination.

FIG. 38. Combination therapy for lung metastatic tumor from 231/ADR cells. administered Ad/gTRAIL aerosolized vector in combination with paclitaxol. Biochemical analysis of cells treated.

### DESCRIPTION OF ILLUSTRATIVE EMBODIMENTS

Cancer gene therapy continues to attract great interest among physicians and researchers alike. Numerous clinical trials continue to be undertaken to test hundreds of possible genetic based tumor therapies. Two potential problems arise in any gene therapy: (a) the expression level of the gene and, (b) toxicity from the gene expression in non-tumor tissues. Obviously, these two issues often work against each other. For example, use of a strong constitutive promoter may be good for generating large amounts of a gene product, but it may prove to be an unacceptable alternative given the associated toxicity, especially if systemic administration is required. Conversely, tissue specific expression can provide for very selective expression of toxic products. However, to date, tissue specific expression has not been successful, largely due to the inability of tissue specific expression systems to generate sufficient levels of therapeutic gene products.

I. The Present Invention

The present invention seeks to address the issues presented above by providing for methods of expressing therapeutic proteins using a new approach to tissue specific gene expression. This involves the use of various tumor cell specific promoters, and the amplification of expression to produce greater amounts of gene products than would be possible using tissue specific promoters alone. At the same time, this system provides for exquisite control of expression, avoiding unwanted toxic effects. In addition, the present invention relies upon this unique expression system to drive the therapeutic genes Bax and TRAIL, both of which have been demonstrated to have toxic effects on non-tumor cells. The details of the invention are provided below.

II. Cell Specific Promoters

In accordance with the present invention, tumor specific promoters may be used in conjunction with an amplifying expression system, described further below. The expression system relies, in the first instance, on the ability of a tissue specific promoter to drive the expression of a transcriptional transactivator, which then turns on a second promoter of interest. In fact, the promoter need not be entirely specific for tumor tissue but, rather, should be active preferentially in tumor tissue. In other words, a small amount of expression in normal tissues, as compared to tumor tissues, may be tolerated. The following tumor specific (or preferential) promoters are contemplated for use in accordance with the present invention.

A. Carcinoembryonic Antigen (CEA) Promoter

CEA is a membrane glycoprotein that is overexpressed in many carcinomas and is widely used as a clinical tumor marker. Paxton et al. (1987); Thompson et al. (1991). Sequence analysis has identified several molecules that are closely related to CEA, including non-specific cross-reacting antigens (NCA) and biliary glycoprotein. Neumaier et al. (1988); Oikawa et al. (1987); Hinoda et al. (1991). CEA is expressed at low levels in some normal tissues and is usually overexpressed in malignant colon cancers and other cancers of epithelial cell origin. Both CEA and NCA expression is fairly homogenous within metastatic tumors, presumably due to the important functional role of these antigens in metastasis. Robbins et al. (1993); Jessup & Thomas (1989).

The cis-acting sequence that confers expression of the CEA gene (SEQ ID NO: 1) on certain cell types has been identified and analyzed. Hauck & Stanners (1995); Schrewe et al. (1990); Accession Nos. Z21818 and AH003050. It consists of approximately 400 nucleotides upstream from the translational start codon and has sequence homology with a similar sequence in NCA. Schrewe et al. (1990). This promoter has been used to drive some suicide genes and to mediate cell killing in tumor xenografts of stably transfected cells. Osaki et al. (1994); Richards et al. (1995). However, its application in gene therapy is limited by its relatively low transcriptional activity. To solve this problem, Kijima et al. recently used the Cre/loxP system to enhance transgene expression from the CEA promoter. Kijima et al. (1999). In their system, a stuffer DNA flanked by a loxP sequence was placed between a transgene and a strong upstream promoter. For coadministration with a second vector expressing a Cre gene driven by a CEA promoter, the stuffer DNA was removed to permit expression of the transgene from its upstream promoter. However, this approach requires rearrangement of vector molecules and is limited by the transcriptional activity of the upstream promoter which could be weak in some cell types.

B. hTERT Promoter

Recently, the human telomerase reverse transcriptase (hTERT) has been cloned by several groups and found to be expressed at high levels in primary tumors and cancer cell lines, but repressed in most somatic tissues. Nakamura et al. (1997); Meyerson et al. (1997); Kilian et al. (1997); Harrington et al. (1997). Data suggest that hTERT is a key determinant of telomerase activity. This includes the finding that hTERT expression is highly correlated with telomerase activity and that ectopic expression of hTERT in telomerase-negative cells is sufficient to reconstitute telomerase activity and extend the life span of normal human cells. Nakamura et al. (1997); Meyerson et al. (1997); Kilian et al. (1997); Harrington et al. (1997); Weinrich et al. (1997); Nakayama et al. (1998); Counter et al. (1998); Bodnar et al. (1998). More recently, it was reported that ectopic expression is required, but not sufficient, for direct tumorigenic conversion of normal human epithelial and fibroblast cells. Hahn et al. (1999).

The promoter region of the hTERT gene also has been cloned. Takakura et al. (1999); Horikawa et al. (1999); Cong et al. (1999); Acession Nos. AB016767 and AF097365. The promoter is high Gly/Cys-rich and lacks both TATA and CAAT boxes, but contains binding sites for several transcription factors, including Myc and Sp1. SEQ ID NO: 2. Deletion analysis of the hTERT promoter identified a core promoter region of about 200 bp upstream of the transcription start site. Transient assays revealed that he core promoter is significantly activated in cancer cell lines but is repressed in normal primary cells.

C. PSA Promoter

Prostate specific antigen (PSA) or KLK3 as it is sometimes called, is a serine protease which is synthesized primarily by both normal prostate epithelium and the vast majority of prostate cancers. Accession No. S81389. The expression of PSA is mainly induced by androgens at the transcriptional level via the androgen receptor (AR). The AR modulates transcription through its interaction with its consensus DNA binding site, GGTACAnnnTGTT/CCT, termed the androgen response element (ARE). Schuur et al. (1996). The core PSA promoter region exhibits low activity and specificity, but inclusion of the PSA enhancer sequence which contains a putative ARE increases expression, specifically in PSA-positive cells. Expression can be further increased when induced with androgens such as dihydrotestosterone. Latham et al. (2000).

D. AFP Promoter

Alpha-fetoprotein (AFP) is expressed at high levels in the yolk sac and fetal liver and at low levels in the fetal gut. Accession No. L34019. AFP transcription is dramatically repressed in the liver and gut at birth to levels that are barely detectable by postnatal day 28. This repression is reversible as the AFP gene can be reactivated during liver regeneration and in hepatocellular carcinomas. Previous studies in cultured cells and trahsgenic mice identified five distinct regions upstream of the AFP gene that control its expression. The promoter and three enhancers functioned as positive regulatory elements, whereas the repressor acted as a negative element. The promoter resides within the 250 bp directly adjacent to exon 1. The repressor, a 600 bp region located between −250 and −850, is required for postnatal AFP repression. Further upstream at −2.5, −5.0 and −6.5 kb are three enhancers termed Enhancer I (EI), EII, and EIII. These three enhancers are active, to varying degrees, in the three tissues where AFP is expressed.

E. Probasin and ARR2PB Promoter

One of the most well-characterized proteins uniquely produced by the prostate and regulated by promoter sequences responding to prostate-specific signals, is the rat probasin protein. Study of the probasin promoter region has identified tissue-specific transcriptional regulation sites, and has yielded a usefuil promoter sequence for tissue-specific gene expression. The probasin promoter sequence containing bases −426 to +28 of the 5′ untranslated region, has been extensively studied in CAT reporter gene assays (Rennie et al., 1993). Prostate-specific expression in transgenic mouse models using the probasin promoter has been reported (Greenberg et al., 1994). Gene expression levels in these models parallel the sexual maturation of the animals with 70-fold increased gene expression found at the time of puberty (2-6 weeks). The probasin promoter (−426 to +28) has been used to establish the prostate cancer transgenic mouse model that uses the fused probasin promoter-simian virus 40 large T antigen gene for targeted over expression in the prostate of stable transgenic lines (Greenberg et al., 1995). Thus, this region of the probasin promoter is incorporated into the 3′ LTR U3 region of the RCR vectors thereby providing a replication-competent MoMLV vector targeted by tissue-specific promoter elements.

The probasin promoter confers androgen selectivity over other steroid hormones, and transgenic animal studies have demonstrated that the probasin promoter will target androgen, but not glucocorticoid, regulation in a prostate-specific manner. Previous probasin promoters either targeted low levels of transgene expression or became too large to be conveniently used. Thus, a probasin promoter was designed that would be small, yet target high levels of prostate-specific transgene expression (Andriani et al., 2001). This promoter is ARR2PB which is a derivative of the rat prostate-specific probasin promoter which has been modified to contain two androgen response elements. ARR2PB promoter activity is tightly regulated and highly prostate specific and is responsive to androgens and glucocorticoids.

III. Transactivating Proteins/Promoters

In one aspect of the invention, the use of various transactivatable promoter systems is described. The basic requirement is that the transactivating element be a single, nucleic acid encoded factor that is functional in a selected target cell. Two particular systems are described below.

A. GAL4-VP16 System

Eukaryotic transcriptional regulatory proteins are typified by the Saccharomyces yeast GAL4 protein, which was one of the first eukaryotic transcriptional activators on which these functional elements were characterized. GAL4 is responsible for regulation of genes which are necessary for utilization of the six carbon sugar galactose. Galactose must be converted into glucose prior to catabolism; in Saccharomyces, this process typically involves four reactions which are catalysed by five different enzymes. Each enzyme is encoded by a GAL gene (GAL 1, 2, 5, 7, and 10) which is regulated by the transactivator GAL4 in response to the presence of galactose.

Each GAL gene has a cis-element within the promoter, termed the upstream activating sequence for galactose (UAS_{G}), which contains 17-basepair sequences to which GAL4 specifically binds. The GAL genes are repressed when galactose is absent, but are strongly and rapidly induced by the presence of galactose. GAL4 is prevented from activating transcription when galactose is absent by a regulatory protein GAL80. GAL80 binds directly to GAL4 and likely functions by preventing interaction between GAL4's activation domains and the general transcriptional initiation factors. When yeast are given galactose, transcription of the GAL genes is induced. Galactose causes a change in the interaction between GAL4 and GAL80 such that GAL4's activation domains become exposed to allow contact with the general transcription factors represented by TFIID and the RNA polymerase II holoenzyme and catalyse their assembly at the TATA-element which results in transcription of the GAL genes.

The functional regions of GAL4 have been carefully defined by a combination of biochemical and molecular genetic strategies. GAL4 binds as a dimer to its specific cis-element within the UASG of the GAL genes. The ability to form tight dimers and bind specifically to DNA is conferred by an N-terminal DNA-binding domain. This fragment of GAL4 (amino acids 1-147) can bind efficiently and specifically to DNA but cannot activate transcription. Two parts of the GAL4 protein are necessary for activation of transcription, called activating region 1 and activating region 2. The activating regions are thought to function by interacting with the general transcription factors. The large central portion of GAL4 between the two activating regions is required for inhibition of GAL4 in response to the presence of glucose. The C-terminal 30 amino acids of GAL4 bind the negative regulatory protein GAL80; deletion of this segment causes constitutive induction of GAL transcription. The VP16 fragment is a transactivation domain from the herpes simplex virus VP16 protein. A fusion product made from the DNA binding portion of GAL4 and VP16 creates a powerful transactivator of appropriate GAL4 promoters.

B. Tetracycline System

Another inducing transactivator system is based on the regulatory elements of a tetracycline-resistance operon, e.g., Tn/10 of E. coli (Hillen & Wissmann, 1989). There, transcription of resistance-mediating genes is negatively regulated by a tetracycline repressor (tetR). In the presence of tetracycline or a tetracycline analogue, tetR does not bind to its operators located within the promoter region of the operon and allows transcription. By combining tetR with a protein domain capable of activating transcription in eucaryotes, such as (i) acidic domains (e.g., the C-termninal domain of VP16 from HSV (Triezenberg et al., 1988) or empirically determined, noneucaryotic acidic domains identified by genetic means (Giniger and Ptashne, 1987) or (ii) proline rich domains (e.g., that of CTF/NF-1 (Mermod et al., 1989)) or (iii) serine/threonine rich domains (e.g., that of Oct-2 (Tanaka and Herr, 1990)) or (iv) glutamine rich domains (e.g., that of Spl (Courey and Tjian, 1988)) a hybrid transactivator is generated that stimulates minimal promoters fused to tetracycline operator (tetO) sequences. These promoters are virtually silent in the presence of low concentrations of tetracycline, which prevents the tetracycline-controlled transactivator (tTA) from binding to tetO sequences. U.S. Pat. No. 5,464,758, which is incorporated by reference, describes the use of this system.

IV. Therapeutic Polypeptides

In accordance with the present invention, one will provide various therapeutic genes for insertion into vector systems, which are then used to deliver the genes to cells and to subjects. Various therapeutic polypeptides are described below.

A. Tumor Suppressors

The tumor suppressor oncogenes function to inhibit excessive cellular proliferation. The inactivation of these genes destroys their inhibitory activity, resulting in unregulated proliferation. The tumor suppressors p53, Rb and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

B. Inducers of Apoptosis

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

C. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occumrng oncogenic growth factor. In one embodiment of the present invention, it is contemplated that antisense or ribozyme construct directed to a particular inducer of cellular proliferation is used to prevent expression of the inducer of cellular proliferation.

The proteins FMS, ErbA, ErbB and Neu are growth factor receptors. Mutations to these receptors result in loss of regulatable function. For example, a point mutation affecting the transmembrane domain of the Neu receptor protein results in the Neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc also are proteins that directly exert their effects on nuclear functions as transcription factors. An extensive list of oncogenes that could be the targets for antisense therapy is present below.

1. Antisense

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair. with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Particular oncogenes that are targets for antisense constructs are ras, myc, neu, raf erb, src, fms, jun, trk, ret, hst, gsp, bcl-2 and abl. Also contemplated to be useful will be anti-apoptotic genes and angiogenesis promoters.

2. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme. Targets for this embodiment will include angiogenic genes such as VEGFs and angiopoeiteins as well as the oncogenes (e.g., ras, myc, neu, raf erb, src, fms, jun, trk, ret, hst, gsp, bcl-2, EGFR, grb2 and abl).

TABLE 1
Oncogenes

| Gene | Source | Human Disease | Function |
|---|---|---|---|
| Growth Factors | | | FGF family member |
| HST/KS | Transfection | | |
| INT-2 | MMTV promoter | | FGF family member |
| | Insertion | | |
| INTI/WNTI | MMTV promoter | | Factor-like |
| | Insertion | | |
| SIS | Simian sarcoma virus | | PDGF B |

Receptor Tyrosine Kinases

| | | | |
|---|---|---|---|
| ERBB/HER | Avian erythroblastosis | Amplified, deleted | EGF/TGF-α/ |
| | virus; ALV promoter | squamous cell | amphiregulin/ |
| | insertion; amplified | cancer; glioblastoma | hetacellulin receptor |
| | human tumors | | |
| ERBB-2/NEU/HER-2 | Transfected from rat | Amplified breast, | Regulated by NDF/ |
| | Glioblatoms | ovarian, gastric cancers | heregulin and EGF- |
| | | | related factors |
| FMS | SM feline sarcoma virus | | CSF-1 receptor |
| KIT | HZ feline sarcoma virus | | MGF/Steel receptor |
| | | | hematopoieis |
| TRK | Transfection from | | NGF (nerve growth |
| | human colon cancer | | factor) receptor |
| MET | Transfection from | | Scatter factor/HGF |
| | human osteosarcoma | | receptor |
| RET | Translocations and point | Sporadic thyroid cancer; | Orphan receptor Tyr |
| | mutations | familial medullary | kinase |
| | | thyroid cancer; | |
| | | multiple endocrine | |
| | | neoplasias 2A and 2B | |
| ROS | URII avian sarcoma | | Orphan receptor Tyr |
| | Virus | | kinase |
| PDGF receptor | Translocation | Chronic | TEL(ETS-like |
| | | myclomonocytic | transcription factor)/ |
| | | leukemia | PDGF receptor gene |
| | | | fusion |
| TGF-β receptor | | Colon carcinoma | |
| | | mismatch mutation | |
| | | target | |

NONRECEPTOR TYROSINE KINASES

| | | | |
|---|---|---|---|
| ABI. | Abelson Mul. V | Chronic myelogenous | Interact with RB, RNA |
| | | leukemia translocation | polymerase, CRK, |
| | | with BCR | CBL |
| FPS/FES | Avian Fujinami SV;GA | | |
| | FeSV | | |
| LCK | Mul. V (murine leukemia | | Src family; T cell |
| | virus) promoter | | signaling; interacts |
| | insertion | | CD4/CD8 T cells |
| SRC | Avian Rous sarcoma | | Membrane-associated |
| | Virus | | Tyr kinase with |
| | | | signaling function; |
| | | | activated by receptor |
| | | | kinases |
| YES | Avian Y73 virus | | Src family; signaling |

SER/THR PROTEIN KINASES

| | | |
|---|---|---|
| AKT | AKT8 murine retrovirus | Regulated by PI(3)K; |
| | | regulate 70-kd S6 k |
| MOS | Maloney murine SV | GVBD; cystostatic |
| | | factor; MAP kinase |
| | | kinase |
| PIM-1 | Promoter insertion | |
| | Mouse | |
| RAF/MIL | 3611 murine SV; MH2 | Signaling in RAS |
| | avian sv | pathway |

MISCELLANEOUS CELL SURFACE

| | | | |
|---|---|---|---|
| APC | Tumor suppressor | Colon cancer | Interacts with catenins |
| DCC | Tumor suppressor | Colon cancer | CAM domains |
| E-cadherin | Candidate tumor | Breast cancer | Extracellular homotypic |
| | Suppressor | | binding; intracellular |
| | | | interacts with catenins |
| PTC/NBCCS | Tumor suppressor and | Nevoid basal cell cancer | 12 transmembrane |
| | Drosophilia homology | syndrome (Gorline | domain; signals |
| | | syndrome) | through Gli homogue |
| | | | CI to antagonize |
| | | | hedgehog pathway |
| TAN-1 Notch | Translocation | T-ALI. | Signaling |
| homologue | | | |

MISCELLANEOUS SIGNALING

| | | | |
|---|---|---|---|
| BCL-2 | Translocation | B-cell lymphoma | Apoptosis |
| CBL | Mu Cas NS-1 V | | Tyrosine- |
| | | | phosphorylated RING |
| | | | finger interact Ab1 |
| CRK | CT1010 ASV | | Adapted SH2/SH3 |
| | | | interact Ab1 |
| DPC4 | Tumor suppressor | Pancreatic cancer | TGF-β-related signaling |
| | | | pathway |
| MAS | Transfection and | | Possible angiotensin |
| | Tumorigenicity | | receptor |
| NCK | | | Adaptor SH2/SH3 |

GUANINE NUCLEOTIDE EXCHANGERS AND BINDING PROTEINS

| | | | |
|---|---|---|---|
| BCR | | Translocated with ABL | Exchanger; protein |
| | | in CML | kinase |
| DBL | Transfection | | Exchanger |
| GSP | | | |
| NF-1 | Hereditary tumor | Tumor suppressor | RAS GAP |
| | Suppressor | Neurofibromatosis | |
| OST | Transfection | | Exchanger |
| Harvey-Kirsten, N-RAS | HaRat SV; Ki RaSV; | Point mutations in many | Signal cascade |
| | Balb-MoMuSV; | human tumors | |
| | Transfection | | |
| VAV | Transfection | | S112/S113; exchanger |

NUCLEAR PROTEINS AND TRANSCRIPTION FACTORS

| | | | |
|---|---|---|---|
| BRCA1 | Heritable suppressor | Mammary | Localization unsettled |
| | | cancer/ovarian cancer | |
| BRCA2 | Heritable suppressor | Mammary cancer | Function unknown |
| ERBA | Avian erythroblastosis | | thyroid hormone |
| | Virus | | receptor (transcription) |
| ETS | Avian E26 virus | | DNA binding |
| EVII | MuLV promotor | AML | Transcription factor |
| | Insertion | | |
| FOS | FBI/FBR murine | | 1 transcription factor |
| | osteosarcoma viruses | | with c-JUN |
| GLI | Amplified glioma | Glioma | Zinc finger; cubitus |
| | | | interruptus homologue |
| | | | is in hedgehog |
| | | | signaling pathway; |
| | | | inhibitory link PTC |
| | | | and hedgehog |
| HMGG/LIM | Translocation t(3:12) | Lipoma | Gene fusions high |
| | t(12:15) | | mobility group |
| | | | HMGI-C (XT-hook) |
| | | | and transcription factor |
| | | | LIM or acidic domain |
| JUN | ASV-17 | | Transcription factor |
| | | | AP-1 with FOS |
| MLL/VHRX + ELI/MEN | Translocation/fusion | Acute myeloid leukemia | Gene fusion of DNA- |
| | ELL with MLL | | binding and methyl |
| | Trithorax-like gene | | transferase MLL with |
| | | | ELI RNA pol II |
| | | | elongation factor |
| MYB | Avian myeloblastosis | | DNA binding |
| | Virus | | |
| MYC | Avian MC29; | Burkitt's lymphoma | DNA binding with |
| | Translocation B-cell | | MAX partner; cyclin |
| | Lymphomas; promoter | | regulation; interact |
| | Insertion avian | | RB; regulate |
| | leukosis | | apoptosis |
| | Virus | | |
| N-MYC | Amplified | Neuroblastoma | |
| L-MYC | | Lung cancer | |
| REL | Avian | | NF-κB family |
| | Retriculoendotheliosis | | transcription factor |
| | Virus | | |
| SKI | Avian SKV770 | | Transcription factor |
| | Retrovirus | | |
| VHL | Heritable suppressor | Von Hippel-Landau | Negative regulator or |
| | | syndrome | elongin; transcriptional |
| | | | elongation complex |
| WT-1 | | Wilm's tumor | Transcription factor |

CELL CYCLE/DNA DAMAGE RESPONSE¹⁰⁻²¹

| | | | |
|---|---|---|---|
| ATM | Hereditary disorder | Ataxia-telangiectasia | Protein/lipid kinase |
| | | | homology; DNA |
| | | | damage response |
| | | | upstream in P53 |
| | | | pathway |
| BCL-2 | Translocation | Follicular lymphoma | Apoptosis |
| FACC | Point mutation | Fanconi's anemia group | |
| | | C (predisposition | |
| | | leukemia | |
| FHIT | Fragile site 3p14.2 | Lung carcinoma | Histidine triad-related |
| | | | diadenosine 5′,3″″- |
| | | | P¹·p⁴ tetraphosphate |
| | | | asymmetric hydrolase |
| hMLI/MutL | | HNPCC | Mismatch repair; MutL |
| | | | homologue |
| hMSH2/MutS | | HNPCC | Mismatch repair; MutS |
| | | | homologue |
| hPMS1 | | HNPCC | Mismatch repair; MutL |
| | | | homologue |
| hPMS2 | | HNPCC | Mismatch repair; MutL |
| | | | homologue |
| INK4/MTS1 | Adjacent INK-4B at | Candidate MTS1 | p16 CDK inhibitor |
| | 9p21; CDK complexes | suppressor and MLM | |
| | | melanoma gene | |
| INK4B/MTS2 | | Candidate suppressor | p15 CDK inhibitor |
| MDM-2 | Amplified | Sarcoma | Negative regulator p53 |
| p53 | Association with SV40 | Mutated >50% human | Transcription factor; |
| | T antigen | tumors, including | checkpoint control; |
| | | hereditary Li-Fraumeni | apoptosis |
| | | syndrome | |
| PRAD1/BCL1 | Translocation with | Parathyroid adenoma; | Cyclin D |
| | Parathyroid hormone | B-CLL | |
| | or IgG | | |
| RB | Hereditary | Retinoblastoma; | Interact cyclin/cdk; |
| | Retinoblastoma; | osteosarcoma; breast | regulate E2F |
| | Association with many | cancer; other sporadic | transcription factor |
| | DNA virus tumor | cancers | |
| | Antigens | | |
| XPA | | xeroderma | Excision repair; photo- |
| | | pigmentosum; skin | product recognition; |
| | | cancer predisposition | zinc finger |

D. Cytokines

Another class of genes that is contemplated to be inserted into the adenoviral vectors of the present invention include interleukins and cytokines. Interleukin 1 (IL-1), IL-2, IL-3, IL-4, IL-5, IL-6, IL-7, IL-8, IL-9, IL-10, IL-I1, IL-12, IL-13, IL-14, IL-15, β-interferon, α-interferon, γ-interferon, angiostatin, thrombospondin, endostatin, METH-1, METH-2, GM-CSF, G-CSF, M-CSF and tumor necrosis factor.

E. Toxins

Various toxins are also contemplated to be useful as part of the expression vectors of the present invention, these toxins include bacterial toxins such as ricin A-chain (Burbage, 1997), diphtheria toxin A (Massuda et al., 1997; Lidor et al., 1997), pertussis toxin A subunit, E. coli enterotoxin toxin A subunit, cholera toxin A subunit and pseudomonas toxin c-terminal. It has been demonstrated that transfection of a plasmid containing the fusion protein regulatable diphtheria toxin A chain gene was cytotoxic for cancer cells. Thus, gene transfer of regulated toxin genes might also be applied to the treatment of cancers (Massuda et al., 1997).

F. Single Chain Antibodies

In yet another embodiment, one gene may comprise a single-chain antibody. Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule.

Single-chain antibody variable fragments (scFvs) in which the C-terminus of one variable domain is tethered to the N-terminus of the other via a 15 to 25 amino acid peptide or linker, have been developed without significantly disrupting antigen binding or specificity of the binding (Bedzyk et al., 1990; Chaudhary et al., 1990). These Fvs lack the constant regions (Fc) present in the heavy and light chains of the native antibody.

Antibodies to a wide variety of molecules are contemplated, such as oncogenes, growth factors, hormones, enzymes, transcription factors or receptors. Also contemplated are secreted antibodies, targeted to serum, against angiogenic factors (VEGF/VSP; βFGF; αFGF) and endothelial antigens necessary for angiogenesis (i.e., V3 integrin). Specifically contemplated are growth factors such as transforming growth factor and platelet derived growth factor.

G. Transcription Factors and Regulators

Another class of genes that can be applied in an advantageous combination are transcription factors. Examples include C/EBPα, IκB, NfκB, Par-4 and C/EBPα.

H. Cell Cycle Regulators

Cell cycle regulators provide possible advantages, when combined with other genes. An example of a regulator that serves to inhibit cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Okamoto et al., 1994; Nobori et al., 1995; Orlow et al., 1999). Restoration of wild-type p16^{INK4} function by transfection with a plasmid expression vector reduced colony formation by some human cancer cell lines (Okamoto, 1994).

Other such cell cycle regulators include p27, p21, p57, p18, p73, p19, p15, E2F-1, E2F-2, E2F-3, p107, p130 and E2F-4. Other cell cycle regulators include anti-angiogenic proteins, such as soluble Flt1 (dominant negative soluble VEGF receptor), soluble Wnt receptors, soluble Tie2/Tek receptor, soluble hemopexin domain of matrix metalloprotease 2 and soluble receptors of other angiogenic cytokines (e.g. VEGFR1/KDR, VEGFR3/Flt4, both VEGF receptors).

I. Chemokines

Genes that code for chemokines also may be used in the present invention. Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Such chemokines include RANTES, MCAF, MIP1-alpha, MIP1-Beta, and IP-10. The skilled artisan will recognize that certain cytokines are also known to have chemoattractant effects and could also be classified under the term chemokines.

V. Treating Subjects With Cancer

Thus, in accordance with the present invention, a cancer patient may be treated with an appropriate gene therapy vector or vectors utilizing tissue preferential promoters in combination with a transactivation system, e.g., tetracycline or GAL4/VP16. Any number of cancers may be treated, for example, brain cancer, head and neck cancer, esophageal cancer, lung cancer, thyroid cancer, stomach cancer, colon cancer, liver cancer, kidney cancer, prostate cancer, breast cancer, cervical cancer, ovarian cancer, testicular cancer, rectal cancer, skin cancer or blood cancer. As discussed below, the constructs and methods of delivery may vary and can be used as appropriate.

A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Initiation Signals and Internal Ribosome Binding Sites

A specific initiation signal may be required for efficient translation of coding sequences. These signals include the ATG initiation codon or adjacent sequences. Exogenous translational control signals, including the ATG initiation codon, also may need to be provided. One of ordinary skill in the art would be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

b. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

c. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997).

d. Termination Signals

The vectors or constructs of the present invention will generally comprise at least one termination signal. A “termination signal” or “terminator” is comprised of the DNA sequences involved in specific termination of an RNA transcript by an RNA. polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

e. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

f. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

g. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

h. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

i. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Thus, nucleic acids of the present invention may be contained a viral vector. Non-limiting examples of virus vectors that may be used to deliver a nucleic acid of the present invention are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a~tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus et al.,, 1994).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al, 1988). Details concerning the generation and use of rAAV vectors are described in U.S. Pat. Nos. 5,139,941 and 4,797,368, each incorporated herein by reference.

3. Retroviral Vectors

Retroviruses have promise as antigen delivery vectors due to their ability to integrate their genes into the host genome, transferring a large amount of foreign genetic material, infecting a broad spectrum of species and cell types and of being packaged in special cell-lines (Miller, 1992).

In order to construct a vaccine retroviral vector, a nucleic acid is inserted into the viral, genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a CDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

B. Pharmaceutical Formulations & Routes of Administration

Pharmaceutical compositions of the present invention comprise an effective amount of one or more genetic constructs dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one vector or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remingtonis Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). Of particular interest is delivery local or regional to a tumor site, circumferential treatment of a tumor site, and treatment of post-operative tumor bed, including catheterization of a body cavity.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. In particular embodiments, prolonged absorption of an injectable composition can be brought about by the use in the compositions of agents delaying absorption, such as, for example, aluminum monostearate, gelatin or combinations thereof.

C. Combination Therapies

In order to increase the effectiveness of a therapy according to the present invention, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that gene therapy according to the present invention can be used similarly in conjunction with chemotherapeutic, radiotherapeutic or immunotherapeutic intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, gene therapy is “A” and the secondary agent, such as radio- or chemotherapy, is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

a. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxol, gemcitabien, navelbine, famesyl-protein tansferase inhibitors, transplatinum, 5-fluorouracil, floxuridine, mutamycin, vincristin, vinblastin and methotrexate, or any analog or derivative variant of the foregoing.

b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

c. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

d. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

e. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Augmenting Transgene Expression from Carcinoembryonic Antigen (CCEA) Promoter via a GAL4 Gene Regulatory System

Materials and Methods

Cell Lines. Human lung cancer cell line A549, colon adenocarcinoma cell lines LoVo and DLD-1, and uterine cervical cancer cell line HeLa were cultured in RPM1 1640 medium or Dulbecco's modified Eagle's medium (DMEM) supplemented with 5-10% FBS, 100 U/ml penicillin, and 100/μg ml streptomycin. Normal human lung fibroblasts (NHFB) were obtained from Clonetics (San Diego, Calif.) and cultured in media recommended by the manufacturer.

Construction and Production of Recombinant Adenovirus Vectors. Several adenoviral vectors were used in this study (Table 2). Ad/CMV-LacZ, Ad/GT-LacZ, Ad/CMV-E1⁻, and Ad/PGK-GV16 had been described previously (Fang et al., 1998; Kagawa et al., 2000b). Ad/CMV-GFP was a gift from Dr. T. J. Liu. Ad/CEA-LacZ and Ad/CEA-GV16 were constructed as described previously (Fang et al., 1997). In brief, pAd/CEA was constructed by replacing the PGK promoter in pAd/PGK with a fragment containing a 424-bp CEA promoter derived from pCEA424/2CAT (Schrewe et al., 1990). Plasmids pAd/CEA-LacZ and pAd/CEA-GV16 were then constructed by inserting LacZ or GV16 cDNA into sites between the CEA promoter and the bovine growth hormone polyadenylation sequence of pAd2/CEA. Recombinant adenovirus was constructed by co-transfecting 293 cells with pAd/CEA-LacZ or pAd/CEA-GV16 along with a 35-kb Cla I fragment from d1324. The recombinants from a single plaque were identified by DNA analysis, expanded in 293 cells, and twice purified by ultracentrifugation. Viral titers were determined by either excitation at A260 nm or TCID₅₀ as described previously (Fang et al., 1998). Titers determined by excitation of A260 nm (particles/ml) were used in subsequent experiments, while titers determined by TCID₅₀ were used as additive information. The ratios of particles to infectious units were usually between 30:1 and 100:1. All viral titers were analyzed by E+virus and endotoxins as described previously (Kagawa et al., 2000a) and were determined to be free of contamination.

TABLE 2
Adenoviral Vectors Employed

| Vector name | Promoter | Gene | Function |
|---|---|---|---|
| Ad/GT-LacZ | GAL4/TATA | LacZ | Augmentation |
| Ad/CEA-LacZ | CEA | LacZ | Basal activity |
| Ad/CEA-GV16 | CEA | GAL4-VP16 | Augmentation |
| Ad/CMV-LacZ | CMV | LacZ | Positive control |
| Ad/PGK-GV16 | PGK | GAL4-VP16 | Positive control |
| Ad/GT-Bax | GAL4/TATA | Bax | Therapeutic |
| Ad/CMV-GFP | CMV | GFP | Vector control |
| Ad/E1 | None | None | Vector control |

In Vitro Gene Transfer. The transduction efficiencies of the adenoviral vectors in A549, DLD1, LoVo, HeLa, and NHFB cells were determined by first infecting the cells with Ad/CMV-LacZ at various MOIs ranging from 500 to 5000 and then staining with the cells with X-gal 24 h after infection. The MOIs that resulted in 50% transduction efficiency were then used to compare CEA promoter activities in the different cell lines. In brief, cells were seeded on 24-well plates at a density of 1×10⁵/well 1 day before infection. A549 and DLD1 cells were infected with a total MOI of 2000, LoVo, NHFB, and HeLa cells were infected with a total MOI of 1000. In cases of coinfection with two vectors, the ratio for the two vectors was fixed at 1:1.

Animal Studies. Animal experiments were carried out in accordance with the Guidelines for the Care and Use of Laboratory Animals (National Institutes of Health publication No. 85-23) and the institutional guidelines of The University of Texas M. D. 15 Anderson Cancer Center. CEA-positive tumors were established by inoculating 1×10⁶ A549 cells into the flanks of adult (6-8 week old) nu/nu mice. Then, when tumors reached 0.5 cm in diameter, usually about 3 weeks after tumor cell inoculation, vectors were injected intratumorally. For reporter gene expression, mice were sacrificed 2 days after vector injection. Tumors, liver, spleen, ovary, brain, kidney, lung, intestine, and heart were harvested for histological and histochemical studies. For antitumor activity of the Bax gene, mice were given three sequential intratumoral injections of 9×10¹⁰ viral particles in a volume of 100 μl per dose. The vector ratio was 1:1 for the binary vector system. Tumor sizes were monitored three times a week. Tumor volumes were calculated as previously described (Gu et al., 2000; Kagawa et al., 2000a).

Biochemical and Histochemical Analysis. For biochemical analysis, cultured cells were lysed and tissues from mice homogenized in β-galactosidase assay buffer. Total protein content was determined using the BCA protein assay kit (Pierce, Rockford, Ill.) according to the manufacturer's instructions. β-Galactosidase activities were determined using the Galacto-Light Plus β-galactosidase assay system (Tropix, Inc., Bedford, Mass.) according to the manufacturer's instructions. Cell viability was determined by staining with tetrazolium dye XTT as described (Kagawa et al, 2000a). For histochemical analysis, tissues and tumors were sectioned and stained with hematoxylin and eosin in the Histology Laboratory of the Department of Veterinary Medicine and Surgery at M. D. Anderson Cancer Center. For X-gal staining, cultured cells or 8-μm frozen sections were fixed with 0.5% glutaraldehyde for 15 min at 4° C.; stained with a solution containing 5 mM K₄Fe(CN)₆, 5 mM K₃Fe(CN)₆, 2 mM MgCl₂, and 1 mg/ml 5-bromo-4-chloro-3-indolyl β-D-galactoside at 37° C. overnight; and then on the next day, counterstained with Nuclear Fast Red (Sigma Chemical Co., St. Louis, Mo.).

Statistical analysis. Differences in transgene expression among the treatment groups were assessed by analysis of variance (ANOVA) using Statistica software (StatSoft, Tulsa, Okla.). For the experiments of tumor growth in vivo, ANOVA with repeated measurement module was used. P≦0.05 was considered significant.

Results

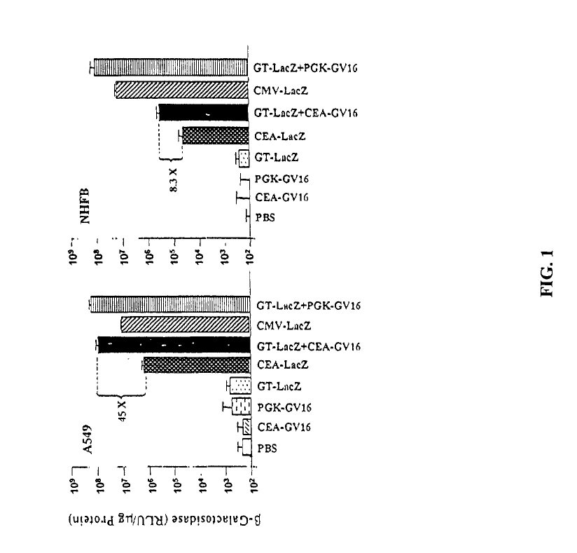

Basal CEA Promoter Levels in CEA-Positive and CEA-Negative Cell Lines. Basal CEA promoter levels were determined in three CEA-positive cell lines (A549, DLD-1, and LoVo), and two CEA-negative cell lines (NHFB and HeLa) by infecting cells with Ad/CEA-LacZ at a dose MOI that resulted in 50% transduction. For A549 and DLD1 cells that dose was 2000 particles/cell; for LoVo, NHLB, and HeLa cells, 1000. Mock-infected cells were used as a background control. Cells infected with the same dose of Ad/E1⁻ or Ad/CMV-LacZ were used as negative or positive controls. β-galactosidase activities were determined 48 h after infection. Ad/E1-infection and mock infection resulted in the same levels of β-galactosidase activities as mock infection (data not shown). The β-galactosidase activities in the CEA-positive cell lines infected with Ad/CEA-LacZ ranged from 1.8×10⁶ RLU/μg to 3×10⁶ (relative light unit) RLU/μg cellular protein. In comparison, β-galactosidase activities in CEA-negative cells infected with Ad/CEA-LacZ were about 4.1-9.5×10⁴ RLU/μg cellular protein (Table 3). Ad/CMV-LacZ infection of the same cell line usually caused 15- to 250-fold higher β-galactosidase activities than did Ad/CEA-LacZ infection (FIG. 1).

In Vitro Augmentation of CEA Promoter by GAL4NVP16. To test whether the transcriptional activity of the CEA promoter could be augmented by the GAL4 gene regulatory system, a binary vector system was used whose first vector (Ad/GT-LacZ) expressed the reporter gene lacZ under the control of a synthetic minimal promoter GT and whose second vector (Ad/CEA-GV16) expressed the transactivating protein GV16 under the control of the CEA promoter. The inventor previously demonstrated that this binary system could effectively induce reporter or transgene expression in vitro and in vivo (Fang et al., 1998; Kagawa et al., 2000b). To determine the levels of β-galactosidase activities in infected cells, cells were infected with Ad/GT-LacZ, Ad/CEA-GV16, Ad/PGK-GV16, Ad/GT-LacZ+Ad/CEA-GV16, or Ad/GT-LacZ+Ad/PGK-GV16. The total MOIs administered in each treatment group were the same as described above. β-galactosidase activities were then determined 48 h after infection. With the exception of HeLa cells treated with Ad/GT-LacZ, cells treated with Ad/CEA-GV16, Ad/PGK-GV16, Ad/GT-LacZ, or the empty vector Ad/E1⁻ expressed the same background levels of β-galactosidase as did cells that were mock infected. β-galactosidase activity in HeLa cells treated with Ad/GT-LacZ was two times higher than the background (P<0.05). The levels of β-galactosidase activity after treatment with Ad/GT-LacZ+Ad/CEA-GV16 or Ad/GT-LacZ+Ad/PGK-GV16 varied among the cell lines. In all cell lines tested, the β-galactosidase activity was significantly higher (p<0.05) after treatment with Ad/GT-LacZ+Ad/CEA-GV16 than after treatment with Ad/CEA-LacZ, although the total dose of LacZ and the CEA promoter in the binary vector system was only half that in the single vector system. Furthermore, the increase in β-galactosidase activities obtained with the binary vector system versus the single vector system was higher in CEA-positive cells (27- to 45-fold) than in CEA-negative cells (6- to 8-fold) (Table 3), suggesting that the GAL4 gene regulatory components not only augmented the levels of transgene expression but also widened the difference in gene expression levels. The mechanisms of this differential augmentation of transgene expression by the GAL4 system were not clear however.

TABLE 3
β-Galactosidase Activity After Infection with Adenoviral Vectors

| | | β-Galactosidase Activity | | −fold |
|---|---|---|---|---|
| | | (RLU/μg protein) | | aug- |
| Cell | | | Ad/GT-LacZ + | men- |
| line | CEA | Ad/CEA-LacZ | Ad/CEA-GV16 | tation |
| A549 | + | 2.0 × 10⁶ ± 4.4 × 10⁵ | 9.1 × 10⁷ ± 1.4 × 10⁷ | 45.5 |
| DLD1 | + | 3.0 × 10⁶ ± 7.9 × 10⁵ | 9.9 × 10⁷ ± 2.5 × 10⁷ | 33 |
| Lovo | + | 1.8 × 10⁶ ± 4.0 × 10⁵ | 4.9 × 10⁷ ± 8.8 × 10⁶ | 27.2 |
| HeLa | − | 9.5 × 10⁴ ± 7.3 × 10³ | 6.2 × 10⁵ ± 2.7 × 10⁵ | 6.5 |
| NHFB | − | 4.1 × 10⁴ ± 2.1 × 10⁴ | 3.4 × 10⁵ ± 9.7 × 10⁴ | 8.3 |

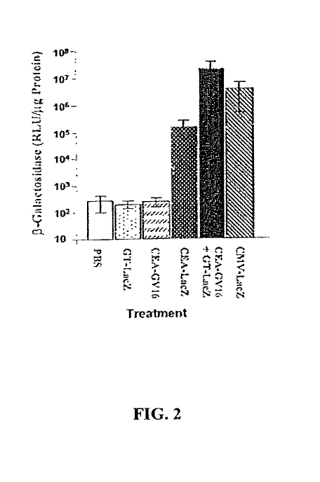

Augmentation of CEA Promoter Expression In Vivo in Established Tumors. Previously, the inventor has shown that use of the binary vector system did not reduce the transduction efficiency of adenoviral vectors in vitro in cultured cells when two vectors were administered at proper ratios (Kagawa et al., 2000b). However, it was not clear whether this would hold true in vivo, where vector dissemination is a major obstacle to efficient transduction and where the absolute requirement that two vectors infect the same target cells might reduce transduction efficiency. To test whether the transcriptional augmentation of the CEA promoter observed in vitro could be similarly elicited in vivo by intratumoral administration, adenoviral vectors (at a fixed total dose of 5×10¹⁰ particles/tumor/mouse) were injected into A549 subcutaneous tumors established in nude mice. When two vectors were used, the vector ratio was 1:1. Mice were killed at 2 days after adenoviral injection, and their tumors and various organs were harvested for biochemical and histochemical analysis of bacterial β-galactosidase activity. As shown by enzymatic analysis of tumor samples, treatment with Ad/CEA-GV16+Ad/GT-LacZ resulted in 2.0×10⁷ RLU/μg cellular protein. In comparison, treatment with Ad/CEA-LacZ resulted in only 1.9×10⁵ RLU/μg cellular protein (FIG. 2). Treatment with Ad/CEA-GV16 or Ad/GT-LacZ alone resulted in only background levels of β-galactosidase. Apparently, the GAL4 gene regulatory system significantly augmented transcriptional activities of CEA promoter in vivo as well (P<0.001). Meanwhile the bacterial β-galactosidase activity in liver, lungs, kidney, spleen, heart, brain, intestine, and ovaries did not exceed background levels in any treatment group, suggesting that little or no vector leaked out of tumors after intratumoral injection.

To test the distribution of transgene expression inside subcutaneous tumors, three sections from each tumor, including the center and 2 mm on either side of the center, were stained with X-gal. Expression of β-galactosidase was detected in the majority of tumor cells treated with either Ad/CMV-LacZ or Ad/CEA-GV16+Ad/GT-LacZ but in less than 30% of tumor cells treated with Ad/CEA-LacZ. Apparently, not only both the total level of transgene expression, but also the percentage of LacZ-positive cells, were augmented by use of the GAL4 gene regulatory system.

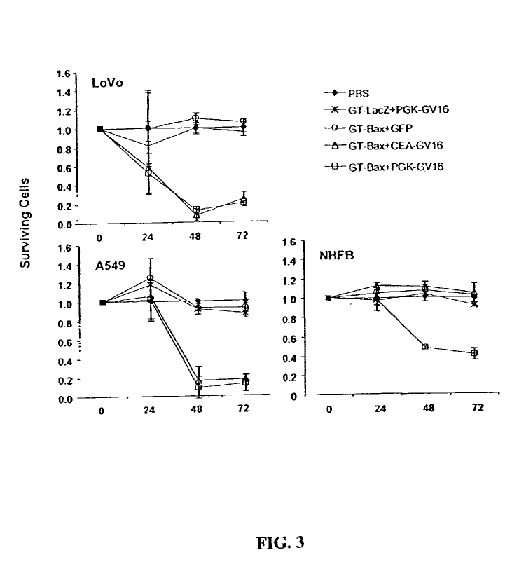

Induction of Cell Death by Ad/CEA-GV16 plus Ad/GT-Bax in CEA-positive Cells. The inventor has developed a binary adenoviral vector system for expression of the pro-apoptotic gene Bax. Ad/GT-Bax+Ad/PGK-GV16 whose administration induced high levels of Bax gene expression, elicited cell death, and suppressed tumor growth in vitro and in vivo (Kagawa et al., 2000a;b). To determine whether co-administration of Ad/CEA-GV16+Ad/GT-Bax would elicit cell death specifically in CEA-positive cells, A549, LoVo, and NHFB cells were infected with this and various other vectors at the fixed total doses described. Cell viability was then monitored by XTT assay over time up to 72 h. While treatment with Ad/PGK-Gv16+Ad/GT-Bax effectively killed cells from all three lines, treatment with Ad/CEA-GV16+Ad/GT-Bax killed only A549 and LoVo cells (FIG. 3). Thus, the use of the GAL4 binary system in combination with the CEA promoter did induce Bax gene-mediated CEA-specific cell death.

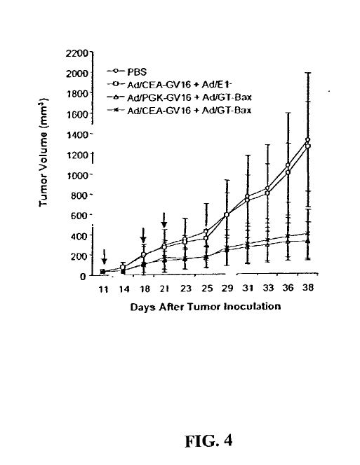

Suppression CEA-Positive Tumor Growth by Ad/CEA-GV16 plus Ad/GT-Bax. To test whether co-administration of Ad/CEA-GV16 plus Ad/GT-Bax will suppress CEA-positive cancer line in vivo, human colon cancer xenografts derived from CEA-positive LoVo cells were established in nude mice. Intratumoral administration of vectors was performed when tumors had reached a diameter of about 0.3-0.5 cm. After three sequential intratumoral injections of adenoviral vectors, animals (5-7 per group) were monitored for tumor growth. Treatment of Ad/CEA-GV16+Ad/GT-Bax or Ad/PGK-GV16+Ad/GT-Bax (positive control) resulted in the same levels of tumor-growth suppression that was significantly different from treatments with PBS or Ad/CEA-GV16+Ad/E1⁻ (P≦0.01) (FIG. 4). This result demonstrated that expression of the Bax gene from the CEA promoter can effectively suppress growth of CEA-positive tumor line. It was also consistent with recent observations that intratumoral administration of binary adenoviral vectors expressing the Bax gene will suppress tumor-growth (Gu et al., 2000; Kagawa et al., 2000a).

### EXAMPLE 2

### Tumor-Specific Transgene Expression from the hTERT Promoter Enables Targeting of the Therapeutic Effects of the Bax Gene to Cancers

Materials and Methods

Construction of recombinant adenovirus vectors. Vectors Ad/E1⁻, Ad/CMV-LacZ, Ad/GT-LacZ, Ad/GT-Bax, and Ad/PGK-GV16 were constructed as described previously (Fang et al., 1997; Kagawa et al., 2000b). Ad/CMV-GFP was provided by Dr. T. J. Liu. To construct Ad/hTERT-LacZ and Ad/hTERT-GV16, plasmid Ad/hTERT-bpA was constructed first by cutting the pGL3-378 plasmid (Takakura et al., 1999) at the Mlu I and Hind III restriction sites and releasing the hTERT core promoter, which was then used to replace the RSV promoter in the shuttle vector Ad/RSV-bpA. Ad/hTERT-LacZ and Ad/hTERT-GV16 were then constructed by placing the LacZ and Ga14/VP16 genes downstream of the hTERT promoter. Recombinant virus from a single plaque was identified by DNA analysis, expanded in 293 cells, and twice purified by ultracentrifugation on a cesium chloride gradient. Virus titers were determined as previously described in Example 1.

Analysis of in vitro gene expression. Human lung cancer cell lines A549 and H1299, and cervical cancer cell line HeLa were originally obtained from ATCC and maintained in the inventor's lab. Human colon cancer cell lines DLD1 and LoVo were obtained from Dr. T. Fujiwara (Okayama University, Japan). Normal human fibroblast (NHFB) cells and normal human bronchial epithelial (NHBE) cells were purchased from Clonetics (San Diego, Calif.) and cultured in media recommended by the manufacturer. Cells were plated 1 day prior to vector infection at densities of 1×10⁵/well in a 24-well plate. Cells were then infected with adenoviral vectors at an MOI (multiplicity of infection) of 1000 viral particles/cell. Twenty-four hours after infection, cells were either stained with X-gal to visualize β-galactosidase expression or harvested for biochemical analysis of β-galactosidase activity.

Biochemical analysis and Cell viability assay. Biochemical analysis was conducted as described in Example 1. For cell viability assays, cells were plated on 96-well plates at 1×10⁴ per well 1 day prior to virus infection. Cells were then infected with adenoviral vectors at a total MOI of 1500 viral particles/cell. Cells were divided into four groups according to the viral vector system given: Ad/CMV-GFP+Ad/PGK-GV16, Ad/GT-Bax+Ad/CMV-GFP, Ad/GT-Bax+Ad/hTERT-GV16 or Ad/GT-Bax+Ad/PGK-GV16. In each group, the ratio of the two viral vectors was 2:1, a ratio shown to be optimal for the induction of transgene expression in previous experiments (Kagawa et al., 2000b). PBS was used for mock infection. The cell viability was determined by XTT assay as described in Example 1. In each treatment group, quadruplicate wells were measured for cell viability at 24, 48, and 72 hr after infection. These experiments were performed at least twice for each cell line.

Apoptosis analysis by flow cytometry. Cells were plated at densities of 1×10⁶/100-mm plate 1 day prior to infection. The cells were then infected with recombinant adenoviral vectors at an MOI of 1500 viral particles/cell. Forty-eight hours later, both adherent and floating cells were harvested by trypsinization, washed with PBS, and fixed in 70% ethanol overnight. Cells were then stained with propidium iodide (PI) for analysis of DNA content. Apoptotic cells were quantified by flow cytometric analysis.

Animal experiments. Animal studies were performed as described in the previous example. In vivo infusion of adenoviral vectors into and subsequent tissue removal from BALB/c mice were done as described in Fang et al, 1997. In the subcutaneous tumor model, 5×10⁶ H1299 cells were inoculated subcutaneously into the dorsal flank of 6- to 8-week-old nude mice (Harlen Sprague Dawley, Indianapolis) to establish tumors. After tumors reached ˜5 mm in diameter, mice were given three sequential intratumoral injections of 9×10¹⁰ viral particles in a volume of 100 μl per dose. Tumor sizes were measured 3 times a week. Tumor volumes were calculated using the formula a×b²×0.5, where a and b represent the larger and smaller diameters, respectively.

Histochemistry study and Analysis of serum AST and ALT. For hematoxylin and eosin (H&E) staining, sectioned tissues or tumors were processed as described in the previous example. For analysis of serum AST and ALT levels, blood was drawn from the tail vein of mice 48 h after adenovirus infusion. The levels of serum AST and ALT were measured as described (Kagawa et al., 2000a). Statistical analysis was conducted as previously described; p≦0.05 was considered significant.

Results

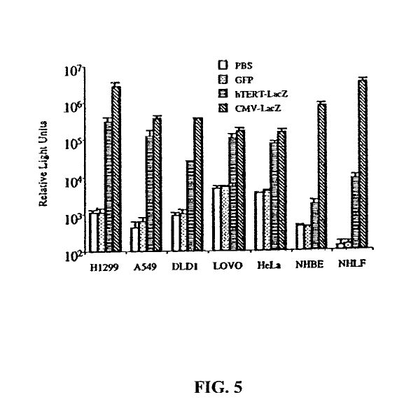

Tumor-specific transgene expression driven by the hTERT promoter in vitro. To assess transgene expression from the hTERT promoter in various cells, the inventor first constructed an adenoviral vector expressing the LacZ gene driven by a 378 bp hTERT core promoter (Takakura et al., 1999). The hTERT promoter activity was assessed in cultured human lung cancer lines cells (H1299 and A549), colon cancer cells (DLD1 and LoVo), cervical cancer cells (HeLa), normal human fibroblast (NHFB) cells, and normal human bronchial epithelial (NHBE) cells by infecting the cells at an MOI of 1000 viral particles. Expression of bacterial β-galactosidase was then analyzed 24 h after infection by either X-gal staining or enzyme assay as described in Methods. In all cancer cell lines tested, both the CMV and hTERT promoters drove strong β-galactosidase expression as evidenced by X-gal staining, while in the two normal cell lines, only infection with Ad/CMV-LacZ produced high levels of transgene expression (nearly 100%). Infection of the normal cells at the same MOI with Ad/hTERT-LacZ resulted in very few LacZ-positive cells. CMV and hTERT promoter activity differed by only 2- to 10-fold in cancer cells compared with more than a 500-fold difference in normal cells (FIG. 5). In all cells tested, hTERT promoter activity was significantly higher in cancer cells than in normal cells (P≦0.05). These results together demonstrated that the hTERT promoter was highly active in a variety of cancer cell lines but not in normal cells, thus suggesting that the hTERT promoter is both strong enough and specific enough to be used in targeting transgene expression to tumors.

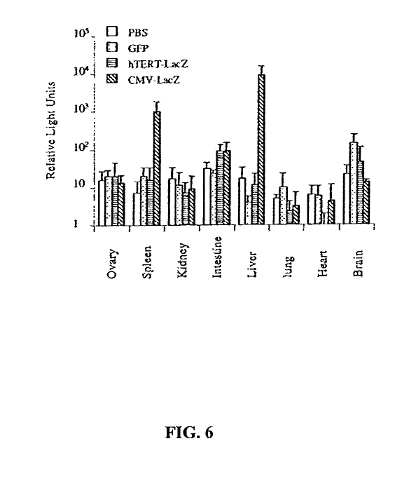

Transcriptional activity of the hTERT promoter in vivo. To investigate the levels of transgene expression induced by the hTERT promoter in vivo, the inventor infused 6×10¹⁰ particles of Ad/hTERT-LacZ, Ad/CMV-LacZ, or Ad/CMV-GFP into BALB/c mice via the tail vein. All mice were euthanized 2 days after vector or PBS infusion; and the liver, spleen, heart, lung, kidney, intestine, ovary, and brain were removed from each for histochemical staining and biochemical analyses of bacterial β-galactosidase expression. High levels of β-galactosidase activity were detected in the livers and spleens of mice treated with Ad/CMV-LacZ. The enzyme activities in other organs of mice treated with Ad/CMV-LacZ were the same as in the background controls. In contrast, the enzyme activities in the livers, spleens, and other organs of mice treated with Ad/hTERT-LacZ were all within the ranges seen in background controls, i.e., PBS- and Ad/CMV-GFP-treated mice (FIG. 6). The failure of the hTERT promoter to drive detectable LacZ expression in adult mouse tissues was not due to the inability of the hTERT promoter to utilize the mouse transcriptional machinery, since a high level of transgene expression was detected in a mouse lung carcinoma cell line (M109) after infection with Ad/hTERT-LacZ (data not shown). Noteworthy is that the promoter region of the mouse TERT gene was also recently cloned; the E-box and two SP1 binding sites in the core promoter region, which were believed to be critical for their high expression in cancer cells, were found to be conserved between hTERT and mTERT (Greenberg et al., 1999). These data together suggest that the hTERT promoter can be used to prevent transgene expression in normal liver and spleen cells and to minimize the liver and spleen toxicity of a therapeutic gene after its systemic delivery.

hTERT promoter-driven Bax gene expression specifically suppresses tumor cells in vitro. The inventor has developed a binary adenoviral vector system that enables us to overcome the difficulties in constructing adenoviral vectors expressing high levels of the strong apoptotic Bax gene (Kagawa et al., 2000a;b). In brief, the system contains two adenoviral vectors. One of these vectors contains a human Bax cDNA under the control of a minimal synthetic promoter comprising five Gal4-binding sites and a TATA box, which is dormant in 293 packaging cells, thus avoiding the toxic effects of the Bax gene on the 293 cells and allowing vector (Ad/GT-Bax) production. The expression of the Bax gene can be induced by co-infecting the Ad/GT-Bax virus with the second adenoviral vector in the binary system (Ad/PGK-GV16). Ad/PGK-GV16 contains a synthetic transactivator, consisting of a fusion protein comprised of a Gal4 DNA-binding domain and a VP16 activation domain under the control of a constitutively active PGK promoter, a housekeeping gene promoter from the mouse 3-phosphoglycerate kinase gene. Previously, it was shown that administration of this binary vector system to cancer cells elicited extensive apoptosis in vitro and suppressed tumor growth in vivo (Kagawa et al., 2000a;b). However, systemic administration of the vector system also resulted in massive apoptosis in the liver, suggesting that overexpression of Bax gene is toxic to normal cells (Kagawa et al., 2000a).

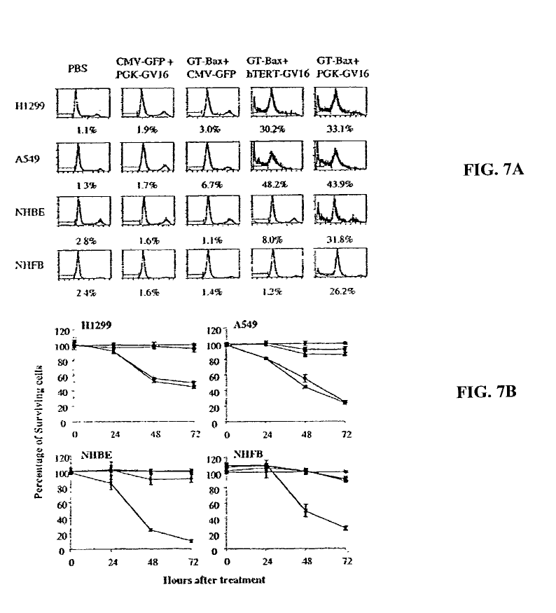

To test whether the hTERT promoter can be used to negate the Bax gene's toxic effects on normal cells while preserving its antitumor activity, the inventor constructed a recombinant adenoviral vector (Ad/hTERT-GV16) by replacing the PGK promoter in Ad/PGK-GV16 with the hTERT promoter. The effects of the Bax gene on normal and tumor cells when induced by the hTERT promoter compared to the PGK promoter were then tested using the binary adenoviral vector system (FIG. 7A). Human lung cancer lines H1299 and A549, NHBE cells, and NHFB cells were treated with PBS, Ad/CMV-GFP+Ad/PGK-GV16, Ad/GT-Bax+Ad/CMV-GFP, Ad/GT-Bax+Ad/hTERT-GV16, or Ad/GT-Bax+Ad/PGK-GV16. The cells were harvested 48 h after the treatment and subjected to FACS analysis to determine the fraction of apoptotic cells by quantifying the sub-G1 population. Induction of apoptosis in H1299 and A549 cells was comparable after infection with either Ad/GT-Bax+Ad/hTERT-GV16 or Ad/GT-Bax+Ad/PGK-GV16, suggesting that the hTERT promoter is as strong as the PGK promoter in inducing Bax gene expression and apoptosis in tumor cells. In the two normal cell lines (NHBE and NHFB), however, treatment with Ad/GT-Bax+Ad/PGK-GV16 elicited substantial apoptosis as well, while treatment with Ad/GT-Bax+Ad/hTERT-GV16 elicited no obvious apoptosis. These results demonstrated that the hTERT promoter can be used to drive tumor-specific proapoptotic gene expression and apoptosis induction while negating the toxicity of a proapoptotic gene to normal cells.

To obtain further evidence that the hTERT promoter could drive specific expression of the Bax gene in tumor cells but not in normal cells, the inventor used the XTT assay to compare cell viability after treatment with either Ad/GT-Bax+Ad/PGK-GV16 or Ad/GT-Bax+Ad/hTERT-GV16. Treatment with either binary vector had comparable cell killing effects on H1299 and A549 cells. However, in NHBE and NHFB cells, treatment with Ad/GT-Bax+Ad/PGK-GV16 also caused dramatic cell loss, while treatment with Ad/GT-Bax+Ad/hTERT-GV16 had only a minimal effect on cell viability (FIG. 7B). The results were further supported by Western blot analysis. Both hTERT and PGK promoter induced strong Bax expression in A549 cells. In comparison, PGK but not hTERT promoter induced strong Bax expression in NHFB cells.

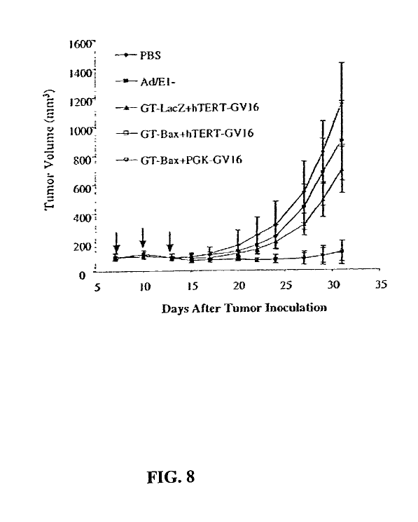

Bax gene expression driven by the hTERT promoter suppresses tumor growth in vivo. To evaluate the possibility of using the hTERT promoter for in vivo Bax gene therapy, the inventor established H1299 tumors subcutaneously in nude mice and treated the tumors with the Bax gene whose expression was driven by the hTERT or PGK promoter. After three sequential intratumoral injections of adenoviral vectors, tumor size changes were monitored for 3 weeks. Treatment with Ad/GT-Bax+Ad/hTERT-GV16 or Ad/GT-Bax+Ad/PGK-GV16 resulted in the same level of tumor growth suppression that were significantly different from treatments with PBS, Ad/E1⁻ or Ad/GT-LacZ+Ad/hTERT-GV16 groups (p≦0.001) (FIG. 8). These results demonstrated that the hTERT promoter can effectively drive transgene expression in tumors in vivo.

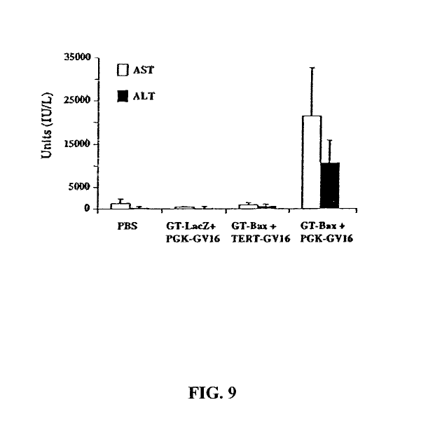

hTERT promoter prevents liver toxicity of the Bax gene. To test whether the hTERT promoter can be used to prevent the toxicity of Bax gene expression in the liver after systemic gene delivery, adult BALB/c or nude mice were infused via the tail vein with PBS, Ad/GT-Bax+Ad/CMV-GFP, Ad/GT-Bax+Ad/hTERT-GV16, or Ad/GT-Bax+Ad/PGK-GV16 at a total dose of 6×10¹⁰ viral particles/mouse. Mice were euthanized 24 h after treatment, and their livers were harvested for histological examination. The majority of hepatocytes underwent apoptosis after PGK promoter-induced Bax expression. In comparison, when Ad/GT-Bax+Ad/hTERT-GV16 was infused, very few apoptotic hepatocytes were observed. To further document the liver toxicity by the Bax gene treatment, blood samples were collected 48 h after intravenous virus injection and the serum levels of liver aspartate transaminase (AST) and alanine transaminase (ALT) were determined (FIG. 9). While treatments with PBS, Ad/GT-LacZ +Ad/PGK-GV16 or Ad/GT-Bax+Ad/hTERT-GV16 resulted in the same serum AST and ALT levels, the treatment with Ad/GT-Bax+Ad/PGK-GV16 resulted over 16- and 41-fold increases in AST and ALT levels, respectively (p≦0.0001). Together, these results suggest that hTERT promoter can be used to prevent the liver toxicity of proapoptotic genes.

### EXAMPLE 3

### Targeted Expression of GFP-TRAIL Fusion Protein from hTERT Promoter Elicits Antitumor Activity Without Toxic Effects on Primary Human Hepatocytes

Methods

Adenoviruses. Adenoviral vectors, Ad/hTERT-LacZ, Ad/hTERT-GV16, Ad/CMV-LacZ, Ad/PGK-GV16, Ad/CMV-GFP, and Ad/GT-TRAIL were described previously (Kagawa et al., 2001; Gu et al., 2000; Fang et al., 1998). Ad/gTRAIL was constructed as described (Fang et al., 1998; Fang et al., 1997). Briefly, an adenoviral shuttle vector (pAd/gTRAIL) was constructed. This vector contains two expression cassettes, one for the GFP/TRAIL fusion protein (Kagawa et al., 2001), whose gene is driven by a synthetic, minimal promoter composed of five sets of GAL4 binding sites and a TATAA sequence (GT promoter), and the other for GAL4NVP16, a transactivator, whose gene is driven by the hTERT promoter. This shuttle plasmid was then cotransfected into 293 cells along with a 35-kb Cla I fragment from adenovirus type 5. Then, recombinant vector Ad/GT-TRAIL was generated by homologous recombination and was plaque-purified. The sequence of its expression cassette was confirmed by automatic DNA sequencing in the DNA sequencing core facility at M. D. Anderson Cancer Center. The expansion, purification, titration, and quality analysis of all vectors used were performed as described in previously examples and in Gu et al., 2000. The titer and yield of Ad/gTRAIL were in the range of other E1-deleted adenoviral vectors.

Cell lines and human hepatocytes. Human lung cancer cell lines, A549 and H460, and human colon cancer cell lines, DLD-1 and Lovo, were grown as described in Example 1. NHPHs were either obtained from Applied Cell Biology Research Institute (Kirkland, Wash.) or isolated from normal, noncirrhotic liver tissues collected from surgical specimens from patients undergoing hepatic resection under a protocol approved by The University of Texas, M. D. Anderson Cancer Center. Collagenase digestion of liver specimens and culturing of primary human hepatocytes were performed as described (Hsu et al., 1985; Strom et al., 1982). Briefly, the liver sample was placed on a sieve over a funnel. Four catheters (connected to the pumping tubes) were inserted into the vessels of liver sample. After residual blood was flushed completely with approximately 500 ml of isolating buffer (8.3 g NaCl, 5 g KCL, 2.4 g HEPES in 1000 ml dH₂O, pH 7.4), the liver sample was perfused with 200 ml of isolating buffer containing 0.5 mg/ml collagenase and 5% bovine serum albumin at 37° C. until the liver started to soften and collapse. Then the sample was torn into small pieces in collagenase solution and filtered through a sieve. Hepatocytes were collected and plated in DMEM medium with 10% FBS and antibiotics for 24 h. The medium of the cells was changed to serum-free medium (Allied Cell Biology Research Institute, Kirkland, Wash.) before the cells were used for experiments.

In vitro gene transfer. The optimal MOI was determined as previously described. MOIs that resulted in 50-80% of cells being stained blue were used in this experiment. These MOIs were 1000 particles for DLD-1, Lovo, A549, NHFB, and primary human hepatocytes and 2000 particles for H460 cells. Unless otherwise specified, Ad/GT-TRAIL+Ad/PGK-GV16 was used as a positive control, and Ad/CMV-GFP was used as a vector control. Cells only treated with PBS were used as a mock control.

Biochemical and flow cytometric assays. β-galactosidase activities were determined using a luminometer and a Galacto-Light Chemiluminescent Assay kit (Tropix, Inc. Bedford, Mass.) as described previously (Gu et al., 2000; Koch et al., 2001). Cell viability was determined by XTT assay as described previously (Kagawa et al., 2000a; Gu et al., 2000). Each experiment was performed in quadruplet and repeated at least twice. Fluorescence-activated cell sorting (FACS) was performed as described previously (Kagawa et al., 2000a;c; Gu et al., 2000). In brief, both adherent and floating cells were harvested at 48 h after treatment. One part was used to analyze GFP expression by determining the percentage of GFP-positive cells through FACS. The other part of the samples was used to quantify apoptotic cells by flow cytometric measuring of cellular DNA content (Kagawa et al., 2000a;c; Gu et al., 2000). Western blot analysis was performed as described (Kagawa et al., 2000a;c; Gu et al., 2000). Antibodies used in this study were anti-caspase-8 (R&D systems, Minneapolis, Minn.), anti-PARP (BD PharMingen, San Diego, Calif.), and anti-GFP (Clontech Inc, Palo Alto, Calif.). Enzyme labeled immunosorbent assay (ELISA) for soluble TRAIL in media of cell cultures was performed as previously described (Kagawa et al., 2001).

Animal Experiments. Animal experiments were carried out as previously described. Human colon carcinoma xenografts were established in nude mice (6-8 w old, Charles River Laboratories Inc. Wilmington, Mass.) by subcutaneous inoculation of 2×10⁶ DLD-1 cells into the dorsal flank of each mouse. Intratumor injection of adenoviral vectors or PBS was performed when the tumors had reached 0.5 cm in diameter. Three intratumoral injections were given every 5 d at a dose of 6×10¹⁰ particles/injection/tumor. Ten mice from each group were followed up by three times per week to measure tumor sizes by calipers. Tumor volume was calculated as volume=a×b²/2 (a: largest diameter, b: smallest diameter) (Kagawa et al., 2001). Mice were sacrificed according to institutional guidelines when the tumor reached 1.5 cm in diameter.

Toxicity after systemic gene delivery also was studied in 6-8 week-old Balb/c mice (Charles River Laboratories Inc.). In brief, mice were given intravenous injections of 6×10¹⁰ particles of adenovirus vectors in a total volume of 200 μl. At 2, 14, and 30 days after injection, 3 mice were sacrificed by CO₂ inhalation. Various organs (brain, heart, lung, liver, intestine, spleen, pancreas, ovary, kidney, and adrenal gland) were then harvested for histopathological examination as described previously (Kagawa et al., 2000a;c; Gu et al., 2000). For liver function analysis, serum samples were collected from mice 2, 10, and 30 days after the treatment. Damage to hepatocytes was monitored by examining serum ALT and AST levels as reported previously (Gu et al., 2000).

TUNEL staining. Tumors were resected at 48 h after intratumoral injection. Apoptosis was assessed by in situ TUNEL staining as reported previously (Kagawa et al., 2001). Briefly, paraffin-embedded sections were deparaffinized and dehydrated. The slides were incubated in 3% H₂O₂ in methanol for 10 min at room temperature and then with 0.02% protease/PBS for 30 min at 37° C. After the slides were incubated for 30 min with reaction buffer containing terminal deoxyribonucleotidyl transferase (TdT) according to the manufacturer's protocol (Roche Molecular Biochemicals), they were stained with ABC reagent. Then, the slides were incubated for 1-2 min with DAB/H2O2 solution washed completely and restained with 4% methyl green.

Statistical analysis. Differences among the treatment groups were assessed by ANOVA using Statistica software (StatSoft Inc., Tulsa, Okla.). Results of the experiments on tumor growth in vivo were analyzed by ANOVA, with a repeated measurement module. P≦0.05 was considered significant.

Results

Augmented transgene expression from the hTERT promoter. The inventor has observed that the hTERT promoter can be used to impose the therapeutic effects of a proapoptotic gene on cancers (Gu et al., 2000). It also was observed that transgene expression from the carcinoembryonic antigen (CEA) promoter can be increased more than 20- to 100-fold in vitro and in vivo via a GAL4 gene regulatory system without loss of the promoter's specificity (Koch et al., 2001). To test whether the levels of transgene expression from the hTERT promoter also can be augmented by using the GAL4 gene regulatory system, LacZ gene expressed directly from the hTERT promoter was compared with that expressed from the hTERT promoter via the GAL4 components after adenovirus-mediated gene transfer. A panel of cell lines was used, including malignant and normal cells for this study. Cells were treated with an adenoviral vector expressing the LacZ gene driven by the hTERT promoter (Ad/hTERT-LacZ) or binary adenoviral vectors consisting of an adenoviral vector containing the LacZ gene driven by the GAL4/TATA promoter (Ad/GT-LacZ) and an adenoviral vector containing hTERT-driving GAL4/VP16 (Ad/hTERT-GV16) fusion gene, whose protein can specifically activate the GAL4/TATA promoter. Cells treated with Ad/CMV-LacZ, Ad/hTERT-GV16, or Ad/GT-LacZ alone were used as treatment controls. Cells treated with PBS were used as a background control.

When cells were treated with binary vectors, the total vector dose remained the same as with the single vectors whereas the ratio for the two vectors was set to 1:1. Levels of LacZ gene expression were determined by X-gal staining and by a β-galactosidase assay. Although treatment with Ad/CMV-LacZ resulted in high levels of LacZ gene expression in all cell lines tested, treatment with Ad/hTERT-LacZ alone or Ad/GT-LacZ+Ad/hTERT-GV16 resulted in high levels of LacZ gene expression only in cancer lines but not in normal fibroblasts. This result is consistent with previous observations that the hTERT promoter can be used for high-level, tumor-specific transgene expression (Gu et al., 2000). On the other hand, cells treated with Ad/GT-LacZ or Ad/hTERT-GV16 alone had only a background level of β-galactosidase, indicating that two vectors are necessary for LacZ gene expression in the binary vector system. Moreover, even though the gene dose in the binary vector system (hTERT and LacZ) was only half that of the single vector system, levels of β-galactosidase activity in cancer cells treated with Ad/GT-LacZ+Ad/hTERT-GV16 were more than 100-fold higher than those in cancer cells treated with Ad/hTERT-LacZ (Table 4). In normal fibroblasts, however, the increase of β-galactosidase activity in the binary vector—treated cells versus single vector—treated cells was about 38-fold, much less than that seen in cancer cells. This result suggests that the GAL4 components can be used to enhance the levels of transgene expression from the hTERT promoter without affecting its specificity.

TABLE 4
β-galactosidase activity after adenovirus-mediated gene transfer *

| | | Ad/hTERT-GV16 + | Augmentation |
|---|---|---|---|
| Cell Lines | Ad/hTERT-LacZ | Ad/GT-LacZ | (Fold) |
| A549 | 6.3 × 10⁴ | 2.2 × 10⁷ | 349 |
| H460 | 3.4 × 10⁴ | 1.3 × 10⁷ | 382 |
| H1299 | 1.5 × 10⁵ | 2.7 × 10⁷ | 180 |
| Lovo | 1.3 × 10⁵ | 2.3 × 10⁷ | 177 |
| NHFB | 2.9 × 10² | 1.1 × 10⁴ | 38 |

* The value represents mean of two triplet assays

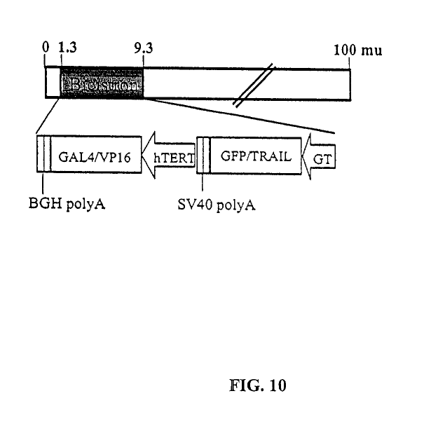

Construction and characterization of Ad/gTRAIL. The ability of the GAL4 gene regulatory components to enhance transgene expression from the hTERT promoter without compromising its specificity led the inventor to design and construct a bicistronic adenoviral vector, Ad/gTRAIL. This bicistronic vector expresses the GFP/TRAIL fusion gene from the hTERT promoter via the GAL4 gene regulatory system (FIG. 10). Although this vector initially was constructed in 293 cells constructed in the inventor's laboratory that expresses trans-repressor GAL4/KRAB-A (Witzgall et al., 1994) (gift of Dr. J. V. Bonventre, Harvard University, Boston), the vector can be expanded and purified from regular 293 cells without any problem.

This vector's functionality was characterized in the human colon cancer cell line DLD 1, which previously was found to be very sensitive to the TRAIL gene (Kagawa et al., 2001). DLD1 cells were infected with a control vector expressing GFP from the cytomegalovirus (CMV) early promoter, Ad/CMV-GFP, at a multiplicity of infection (MOI) of 1000 viral particles (vp)/cell. This resulted in about 50% GPF positive cells, as detected under fluorescence microscope but did not kill the cells. However, infecting these cells with the same dose of Ad/gTRAIL resulted in the same level of GFP-positive cells, yet, more than 90% of the Ad/gTRAIL-treated cells were killed 48 h after treatment, as judged by morphology changes. Thus, treatment with Ad/gTRAIL can effectively kill TRAIL-sensitive cancer cells.

Transgene expression and apoptosis induction by Ad/gTRAIL in cancer cells in vitro. To further document the levels of transgene expression and the antitumor activity of Ad/gTRAIL, human lung cancer cell lines (A549 and H460) and human colon cancer cell lines (DLD-1 and Lovo) were treated with Ad/gTRAIL, Ad/CMV-GFP, or Ad/GT-TRAIL+Ad/PGK-GV16 at a fixed total MOI as described in Materials and Methods. Cells treated with PBS were used as a mock control. Two days later, cells were harvested and divided into two parts. One part was used to analyze GFP expression, and the second part was used to quantify apoptosis; both analyses were performed by flow cytometric assay. Treatment with Ad/CMV-GFP or Ad/gTRAIL resulted in similar levels of GFP-positive cells (70% to 90%) in all the cell lines tested, suggesting that levels of transgene expression for the two vectors were similar in these cancer cell lines (FIG. 11A). However, treatment with Ad/gTRAIL dramatically increased apoptotic cells, a result that is comparable to findings for cells treated with a binary vector system expressing wild-type human TRAIL (Ad/GT-TRAIL+Ad/PGK-GV16) (Kagawa et al., 2001). In comparison, treatment with Ad/CMV-GFP resulted in only background levels of apoptosis, and treatment with Ad/GT-TRAIL+Ad/PGK-GV16 resulted in only background levels of GFP-positive cells because the binary vectors did not contain any GFP component. These results demonstrate that treatment with Ad/gTRAIL can elicit high levels of transgene expression and high levels of apoptosis in cancer cells.

The anti-tumor activity of Ad/gTRAIL was further documented with the XTT assay (FIG. 11B). Treatment of DLD-1, Lovo, A549, and H460 by Ad/gTRAIL or Ad/GT-TRAIL+Ad/PGK-GV16 dramatically reduced the survival of cells in vitro. These results are significantly different from those associated with cells treated with Ad/CMV-GFP or mock control. These results also were supported by Western blot analysis for apoptotic markers. Caspase-8 and poly (ADP—ribose) polymerase (PARP) cleavage occurred in groups treated with Ad/gTRAIL or Ad/GT-TRAIL+Ad/PGK-GV16 as early as 12 h after infection but not in the Ad/CMV-GFP-treated group or in mock controls.

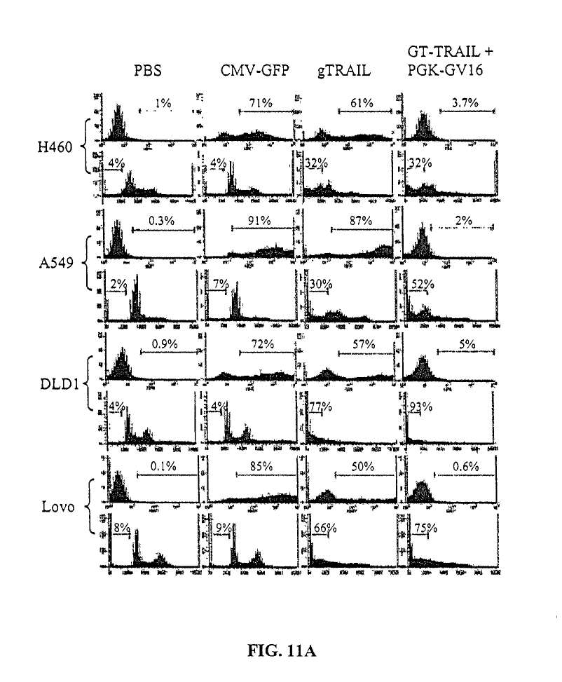

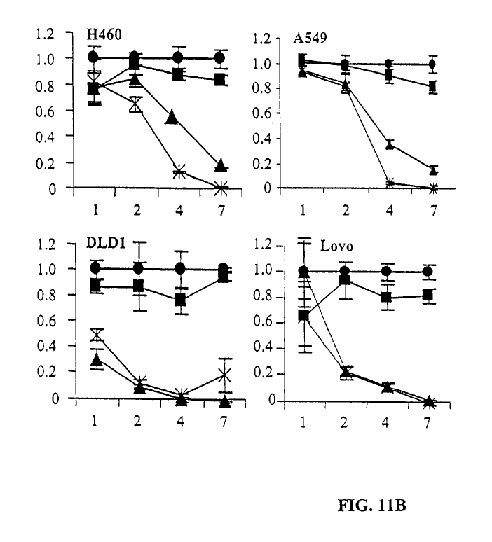

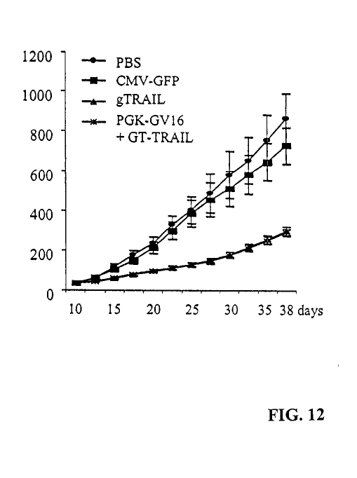

Suppression of tumor growth by Ad/gTRAIL in vivo. Direct intratumoral administration of the binary adenoviral vectors expressing the human TRAIL gene suppressed DLD1 tumor growth in vivo in nude mice (Kagawa et al., 2001). To test whether intralesional administration of Ad/gTRAIL also can suppress tumor growth, the antitumor effect of Ad/gTRAIL was compared with that of the binary vectors (FIG. 12). A direct comparison showed that intralesional administration of Ad/gTRAIL resulted in the same antitumor effects as those of Ad/GT-TRAIL+Ad/PGK-GV16, suggesting that Ad/gTRAIL is as effective as Ad/GT-TRAIL+Ad/PGK-GV16 in terms of antitumor activity in vivo. In comparison, tumors treated with Ad/CMV-GFP grew as fast as those treated with PBS. Post-treatment histochemical examination of tumor tissues supported these results. Treatment with Ad/gTRAIL or Ad/GT-TRAIL+Ad/PGK-GV16 dramatically increased apoptosis, whereas treatment with Ad/CMV-GFP or PBS resulted in only background apoptosis.

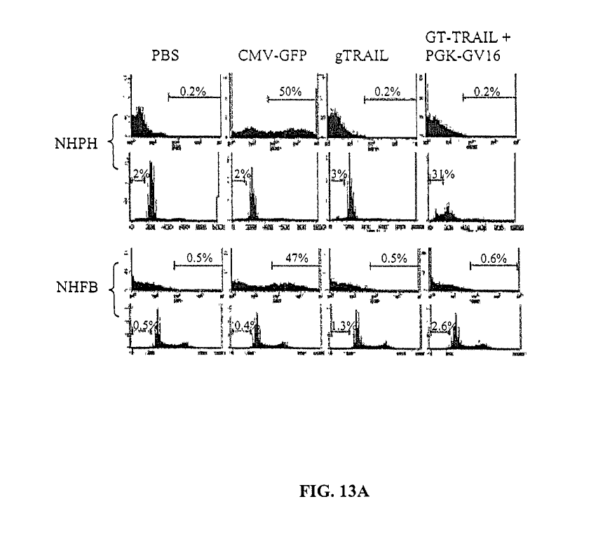

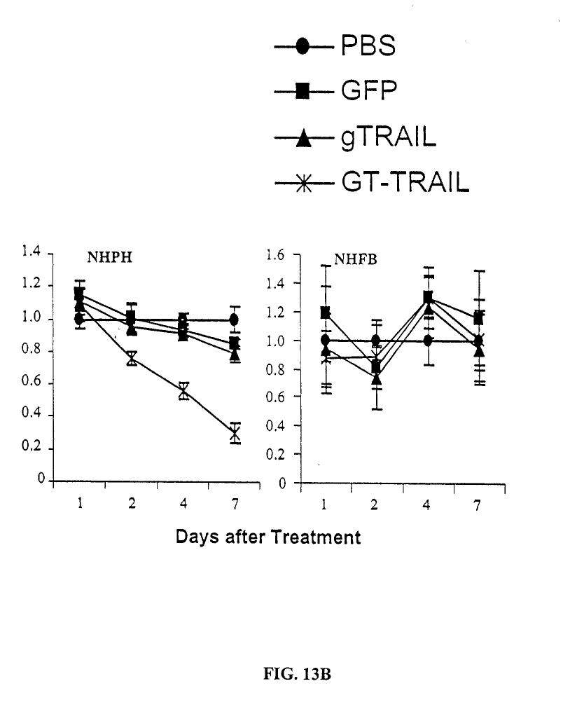

Transgene expression and toxicity Ad/gTRAIL in normal human primary hepatocytes. The effects of Ad/gTRAIL on normal human fibroblasts (NHFBs) or NHPHs isolated from surgical specimens were evaluated. For this purpose, NHPHs or NHFBs were treated either with PBS or with Ad/CMV-GFP, Ad/gTRAIL, or Ad/GT-TRAIL+Ad/PGK-GV16 at a total MOI of 1000 vp/cell. When the binary system was used, the total dose remained the same while the ratio for two vectors was set to 1:1. Two days later, cells were harvested and divided into two parts. One part was used to analyze GFP expression, and the second part was used to quantify apoptotic cells by flow cytometric assay, as described above. Only treatment with Ad/CMV-GFP resulted in more than 50% of GFP-positive cells in either NHPHs or NHFBs. In contrast, treatment with Ad/gTRAIL resulted in less than 1% of GFP-positive cells, similar to what is seen in NHPH or NUFB cells treated with PBS or Ad/GT-TRAIL+Ad/PGK-GV16. (The latter does not have a GFP component; therefore, transgene expression can not be detected by GFP assay) (FIG. 13A). Cytometric analysis of apoptosis showed that treatment with Ad/gTRAIL or Ad/GT-TRAIL+Ad/PGK-GV16 resulted in only background levels of apoptosis in fibroblasts. This finding is consistent with the inventor's previous observation that treatment of the TRAIL-expressing vectors did not result in cell death in NHFBs. Interestingly, however, treatment with Ad/GT-TRAIL+Ad/PGK-GV16 led to a dramatic increase in apoptotic cells (more than 30%) in NHPHs. In comparison, treatment with Ad/gTRAIL resulted in only a background level of cell death similar to that seen in cells treated with PBS or control vector (FIG. 13A). These data indicate that NHPHs are susceptible to full-length human TRAIL molecules and that the hTERT promoter can be used to prevent expression of therapeutic genes in normal human hepatocytes, thereby preventing possible toxicity. This observation was further supported by the fact that treatment of NHPHs with Ad/GT-TRAIL+Ad/PGK-GV16, but not Ad/gTRAIL, Ad/CMV-GFP, or Ad/GT-LacZ+Ad/PGK-GV16, resulted in typical apoptotic morphological changes as revealed by microscopic study or cell viability loss as revealed by XTT assay (FIG. 13B). The same results were observed when using primary hepatocytes obtained from Applied Cell Biology Research Institute (Kirkland, Wash.).

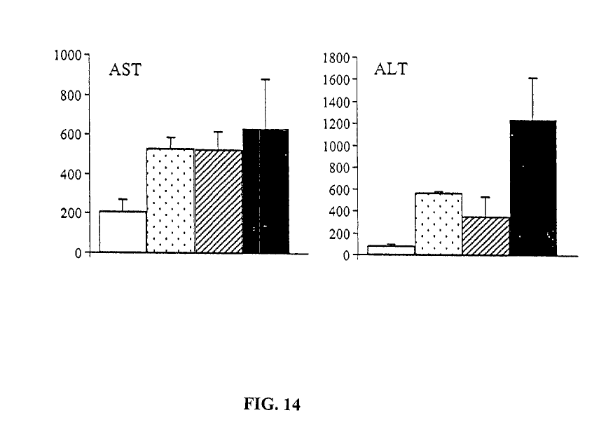

Transgene expression and toxicity of Ad/gTRAIL after systemic administration. The inventor also investigated levels of transgene expression in the liver and the possible toxicity of Ad/gTRAIL after systemic administration. For this purpose, adult Balb/c mice (6-8 old) were infused with PBS, Ad/CMV-GFP, Ad/gTRAIL, and Ad/GT-TRAIL+Ad/PGK-GV16 via the tail vein (ratio 1:1 in this group) at a total dose of 6×10¹⁰ particles/mouse. The inventor's previous study had shown that more than 90% of liver cells are transduced at this dose (Gu et al., 2000). Animals were sacrificed at 2, 14, and 30 days after injection. Liver, spleen, lung, heart, pancreas, kidney, intestine, gonad, and brain were harvested for histopathological examination. No significant microscopic lesions were observed in any animals at 2 days after treatment. By 2 weeks, all animals treated with adenoviral vectors showed lymphoid hyperplasia in the spleen and inflammatory cell (lymphocytes, plasma cells, and neutrophils) infiltration in some portal areas in the liver. In addition, animals treated with Ad/GT-TRAIL+Ad/PGK-GV16 showed scattered necrotic hepatocytes and had numerous binucleated or trinucleated hepatocytes (polyloidy) and hepatocytes with large irregular-shaped nuclei (karyomegaly). Changes in hepatocytes were recovered by day 30. The results of serum liver enzyme assays were consistent with histopathological changes observed in the liver. Aspartate transaminase (AST) and alanine transaminase (ALT) levels were within normal ranges at day 2 and day 30, but were elevated at day 14 in animals treated with adenoviral vectors (FIG. 14). The elevation was more pronounced in animals treated with Ad/GT-TRAIL+Ad/PGK-GV16. Of note, E1-deleted adenoviral vectors are immunogenic and will cause a subacute inflammatory response in the livers of immunocompetent animals after systemic delivery (Ji et al., 1999; Yang et al., 1994). Because a similar degree of inflammatory response was observed in animals treated with Ad/CMV-GFP or Ad/gTRAIL, this response is regarded as vector related rather than as transgene related. Interestingly, the inflammatory response was more severe in animals treated with Ad/GT-TRAIL+Ad/PGK-GV16. The significance of this finding is not yet clear, however, this phenomenon was not observed in nude mouse (Kagawa et al., 2001).

Because systemic administration of adenoviral vector namely resulted in transduction of liver cells (Gu et al., 2000; Fang et al., 1994), liver samples from the above animals also were collected for Western blot analysis of GFP or GFP/TRAIL fusion protein expression and for polymerase chain reaction (PCR) analysis of the viral genome. For animals treated with Ad/CMV-GFP, Western blot analysis with anti-GFP polyclonal antibody showed a strong GFP band by day 2 that became much weaker by day 14 (data not shown). The GFP was not detectable by Western blot by day 30. This result is consistent with observations that transgene expression from adenoviral vectors is transient in immunocompetent animals (Fang et al., 1994; 1995; 1996). In animals treated with Ad/gTRAIL, however, no transgene expression was detected by Western blot analysis in any animals at any of the time points tested. Of note, the GFP antibody used can readily detect the GFP/TRAIL fusion protein in DLD1 cells treated with Ad/gTRAIL. Furthermore, the same level of viral DNA was detected in the livers of animals treated with either Ad/CMV-GFP or Ad/gTRAIL by a semi-quantitating PCR analysis, suggesting that the lack of transgene expression by Ad/gTRAIL was not caused by an artifact. These findings suggests that the hTERT promoter can be used to prevent expression of the GFP/TRAIL gene in the liver after systemic adminstration of Ad/gTRAIL. This is consistent with the inventor's previous observation that the hTERT promoter can be used to prevent Bax gene-related liver toxicity after systemic administration of the Bax-expressing binary adenoviral vectors (Gu et al., 2000).

### EXAMPLE 4

### Treatment with the GFP-TRAIL Fusion Gene Expressed from the hTERT Promoter in Breast Cancer Cells

Materials and Methods

Cell Lines and Reagents. Human breast cancer cell lines MCF7, MDA-MB-231, MDA-MB-453, and MDA-MB-468 were grown in RPMI 1640 medium supplemented with 10% heat-inactivated fetal bovine serum, and antibiotics, and glutamine. Immortalized nontransformed breast epithelial cell lines MCF10A and MCF10F and normal human mammary epithelial cells (NHMEC) were purchased from Clonetics (San Diego, Calif.). Primary normal human mammary epithelial cells (PNHMEC) isolated from normal breast tissue (Stampfer and Yaswen, 1994) were provided by Dr. Yinhua Yu (The University of Texas M. D. Anderson Cancer Center). Doxorubicin-resistant MDA-MB-231 cells were obtained through the stepwise exposure of the parental cells to doxorubicin. In brief, the parental MDA-MB-231 cells were treated with doxorubicin (Ben Venue Labs, Inc., Bedford, Ohio) at concentration of 2 μM (1.16 μg/ml, which is the IC₈₀ of MDA-MB-231 cells). Most cells were killed after day 4 of treatment. The residual surviving cells were then allowed to grow in fresh medium. When these residual cells reached 70-80% confluence in the plate, they were treated again with the same concentration of doxorubicin. After this cycle was repeated several times, the concentration of doxorubicin was increased multiple times followed by the regrowth of cells. Finally, MDA-MB-231 resistant to doxorubicin at a concentration of 16 μM were selected (designated 231/ADR) and used in the subsequent experiments. Other agents used in these experiments included gemcitabine (Eli Lilly and Co., Indianapolis, Ind.), vinorelbine (Pierre Fabre, Idron 64320, France), paclitaxel (Bristol-Myers Squibb Co., Princeton, N.J.), irinotecan (Pharmacia & Upjohn Co., Kalamazoo, Mich.), floxuridine (Ben Venue Labs, Inc., Bedford, Ohio), and TRAIL protein (R & D Systems, Minneapolis, Minn.).

Adenovectors. Adenovectors Ad/CMV-LacZ, Ad/PGK-GV16, Ad/CMV-GFP, Ad/GT-TRAIL and Ad/gTRAIL have been described previously (Gu et al., 2000; Kagawa et al., 2001; Zhang et al., 2002). The expansion, purification, titration, and quality analysis of all vectors used were performed as described in previous examples. The MOIs used in this experiment were those that resulted in 50-80% of cells being stained blue. The MOIs for each of the cell lines were as follows: 2000 particles for MDA-MB-468, 4000 particles for MDA-MB-231 and MDA-MB-453, 8000 particles for MCF7, respectively. The MOI of MCF-10A, MCF10F and NHME were 4000 particles. Unless otherwise specified, Ad/CMV-GFP was used as a vector control and PBS as a mock control.

Assays. Cell viability, flow cytometry, and Western blot analysis were performed as described in previous examples. See also Gu et al., 2000 and Kagawa et al., 2001. Biochemical analysis, using β-galactosidase was determined as dexcribed in Example 1. For Western analysis anti-caspase-8 (R&D systems, Minneapolis, Minn.), anti-caspase-3 (BD PharMingen, San Diego, Calif.), anti-PARP (BD PharMingen), anti-GFP (Clontech Inc., Palo Alto, Calif.), and anti-hTRAIL (Alexis, San Diego, Calif.) antibodies were used. Apoptosis in tumors was assessed by in situ TUNEL staining as decribed in Example 3.

Animal Experiments. Animal experiments were carried out as described in the previous examples. Human breast cancer xenografts were established in 6 to 8 week old nude mice (Charles River Laboratories Inc., Wilmington, Mass.) by subcutaneous inoculation of 5×10⁶ MDA-MB-231 or 231/ADR cells into the dorsal flank of each mouse. The levels of serum AST and ALT were measured as described in example 2 and in Gu et al., 2000 and Kagawa et al., 2001.

Statistical Analysis. Differences among the treatment groups were assessed as described in previous examples. Survival was assessed using the Kaplan-Meier method. The drug concentration that inhibited cells growth by 50% (IC₅₀) was calculated by CurveExpert 1.3 software (Starkville, Miss.).

Results

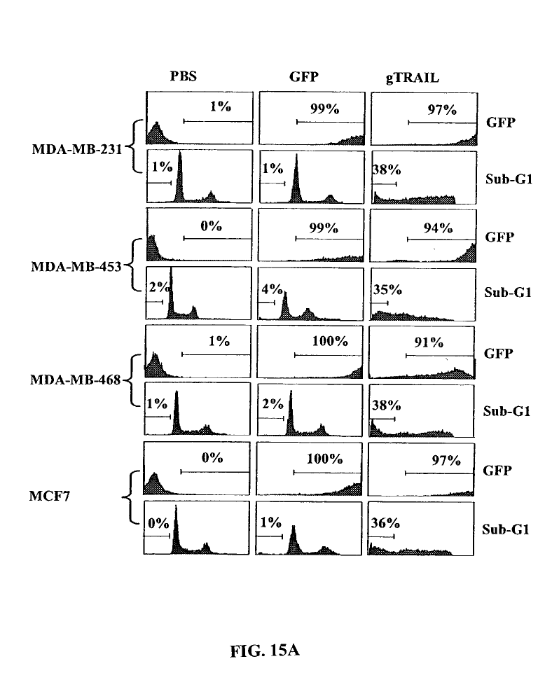

Transgene expression and apoptosis induction by Ad/gTRAIL in vitro in breast cancer cells. To test the levels of transgene expression and the antitumor activity of Ad/gTRAIL, four human breast cancer cell lines (MDA-MB-231, MDA-MB-453, MDA-MB-468, and MCF7) were treated with Ad/gTRAIL and Ad/CMV-GFP at a fixed total MOI, as described previously. Cytometry was used to analyze GFP expression, and quantify apoptosis. Treatment with Ad/CMV-GFP or Ad/gTRAIL resulted in similar levels of GFP-positive cells (70%-90%) in all cell lines (FIG. 15A). However, only treatment with Ad/gTRAIL significantly increased the number of apoptotic cells. The induction of apoptosis by Ad/gTRAIL was confirmed by Western blot analysis. Caspase-8, caspase-3, and PARP cleavage were observed only in cells treated with Ad/gTRAIL or Ad/GT-TRAIL+Ad/PGK-GV16, a binary vector system expressing wild type TRAIL cDNA from a PGK promoter 24. The results were validated by cell viability analysis. Treatment of the cancer cell lines with Ad/gTRAIL significantly reduced cell viability compared with cells treated with Ad/CMV-GFP or PBS (FIG. 15B). Together, these results suggest that the cancer cell lines tested were highly susceptible to Ad/gTRAIL.

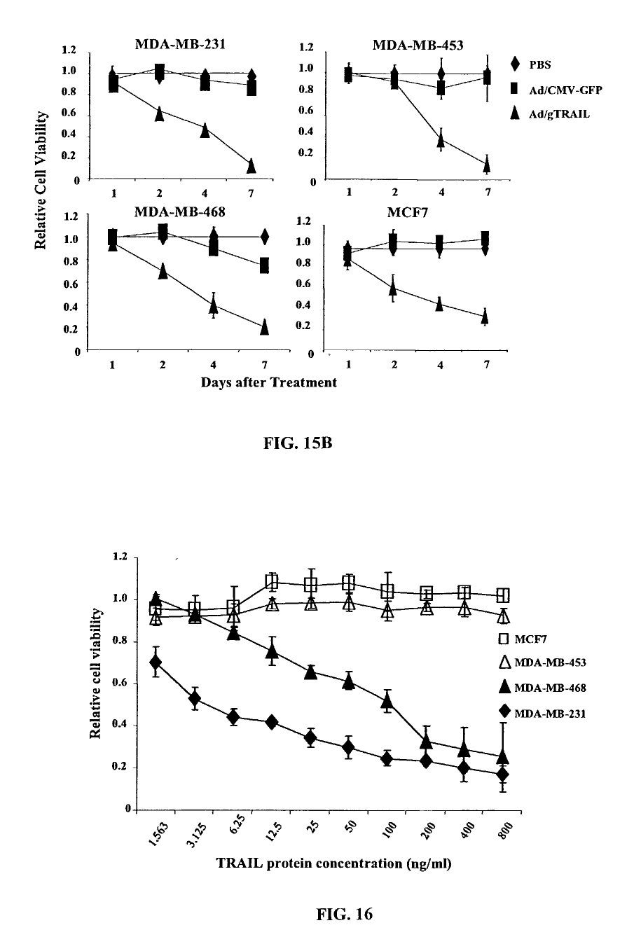

To test whether these cells were similarly sensitive to soluble TRAIL protein, cells were treated with recombinant TRAIL protein at various concentrations up to 800 ng/ml. Cell viability was then determined at 24, 48, and 96 h after treatment. While MDA-MB-231 and MDA-MB-468 were sensitive to TRAIL protein at various concentrations, MDA-MB-453 and MCF7 cells were resistant to TRAIL protein even at a concentration of 800 ng/ml (FIG. 16). Interestingly, however, these two cell lines were as sensitive to Ad/gTRAIL as the other cell lines. Although the mechanisms responsible remain to be characterized, this result suggests that membrane-bound TRAIL may be more effective than soluble TRAIL in certain cells.

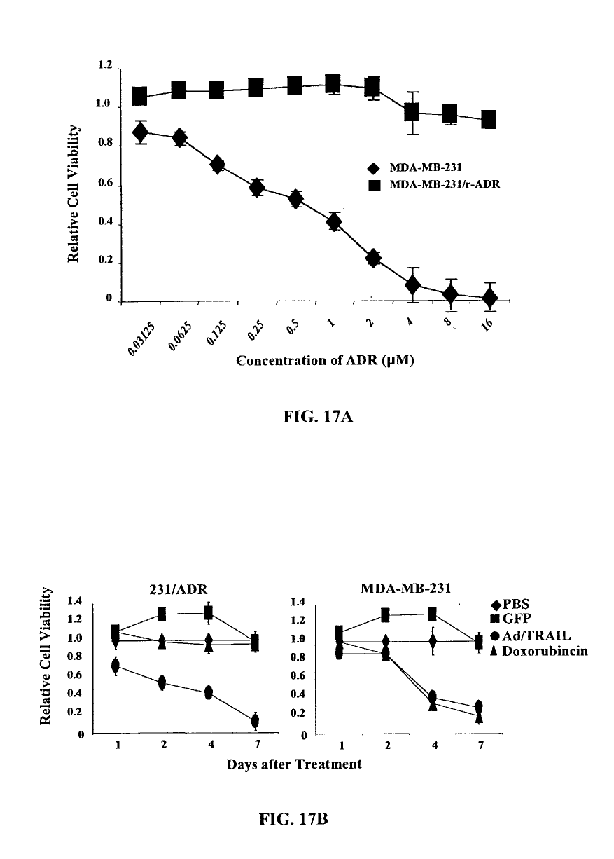

Effects of Ad/gTRAIL on doxorubicin resistant cancer cells. To test whether Ad/gTRAIL is also effective in cancers resistant to chemotherapy, MDA-MB-231 cells were repeatedly treated with doxorubicin and selected doxorubicin-resistant cells (231/ADR cells). While parental MDA-MB-231 cells were susceptible to doxorubicin at 2 μM, 231/ADR was resistant to doxorubicin at a concentration of 16 μM (FIG. 17A). Indeed, a subsequent study showed that the IC₅₀ of a variety of chemotherapeutic agents was increased in 231/ADR cells as compared with parental MDA-MB-231 cells, suggesting that 231/ADR cells are also relatively resistant to many chemotherapy drugs (Table 5).

TABLE 5
IC₅₀ of different chemotherapeutic agents and Ad/gTRAIL in parental and
Doxorubicin-resistant MDA-MB-231 cells.

IC₅₀

| Agents | MDA-MB-231 | 231/ADR |
|---|---|---|
| Doxorubicin (μM) | 1.14 | 97.63 |
| Paclitexol (nM) | 3.46 | 16.93 |
| Vinorelbine (nM) | 2.40 | 22.94 |
| Irinotecan (μM) | 1.54 | 5.94 |
| Floxuridine (μM) | 0.10 | 4.70 |
| Gemcitabine (nM) | 18.63 | 36.97 |
| Ad/gTRAIL (vp/cell) | 1994 | 1211 |

The ability of Ad/gTRAIL to induce transgene expression and apoptosis in MDA-MB-231 and 231/ADR cells was analyzed. Flow cytometry showed that levels of transgene expression and apoptosis induced by Ad/gTRAIL were similar in 231/ADR and MDA-MB-231 parental cells. And, these results were supported by the XTT assay (FIG. 17B). The 231/ADR cells were as sensitive to Ad/gTRAIL as the parental cells, suggesting that Ad/gTRAIL is useful for the treatment of cancers resistant to conventional therapy.

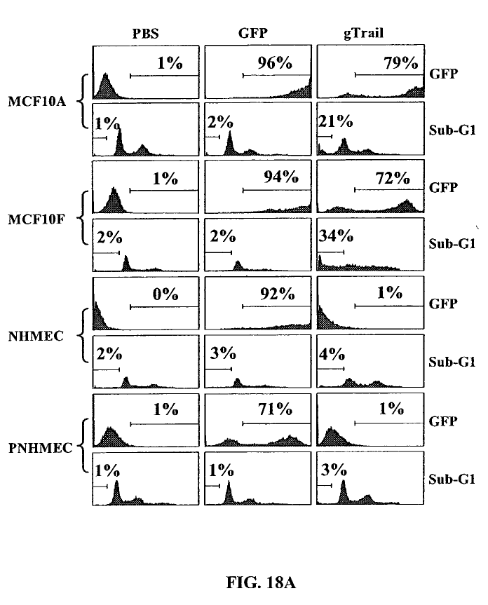

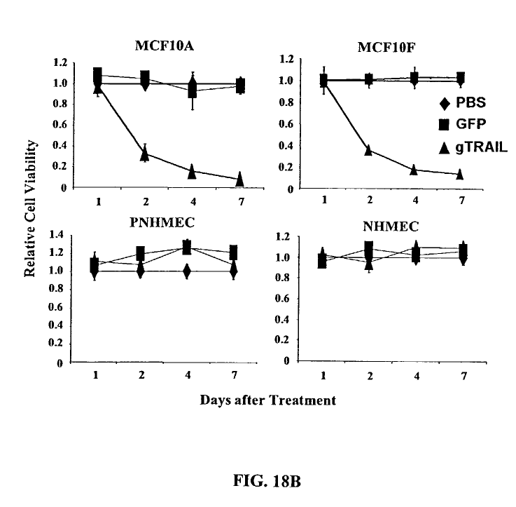

Effects of Ad/gTRAIL on normal cells. The inventors were also interested in the ability of Ad/gTRAIL to induce transgene expression and apoptosis in the immortalized nontransformed breast epithelial cell lines MCF10A and MCF10F, normal human mammary epithelial cell (NHMEC), and primary normal human mammary epithelial cells (PNHMEC) isolated from surgical specimens. More than 70% of NHMEC and PNHMEC treated with Ad/CMV-GFP were positive for GFP, whereas only 1% of cells treated with Ad/gTRAIL were positive for GFP. However, only background levels of apoptosis were observed in these two cells after treatment with either Ad/CMV-GFP or Ad/gTRAIL. This finding is consistent with recent observation that the hTERT promoter is minimally active in normal human cells (Gu et al., 2000). Interestingly, however, Ad/gTRAIL induced substantial levels of transgene expression and apoptosis in both MCF10A and MCF10F, the two of immortalized nontransformed breast cell lines (FIG. 18A). This result is consistent with observation in 293 cells, an immortalized human kidney epithelial cell line that has high hTERT promoter activities (Gu et al., 2000) and is sensitive to TRAIL (Kagawa et al., 2001). Cell viability analysis also supported the results from the cytometry assay (FIG. 18B). In summary, therefore, treatment with Ad/gTRAIL elicited cell death in MCF10A and MCF10F cells but not in NHMEC and PNHMEC.

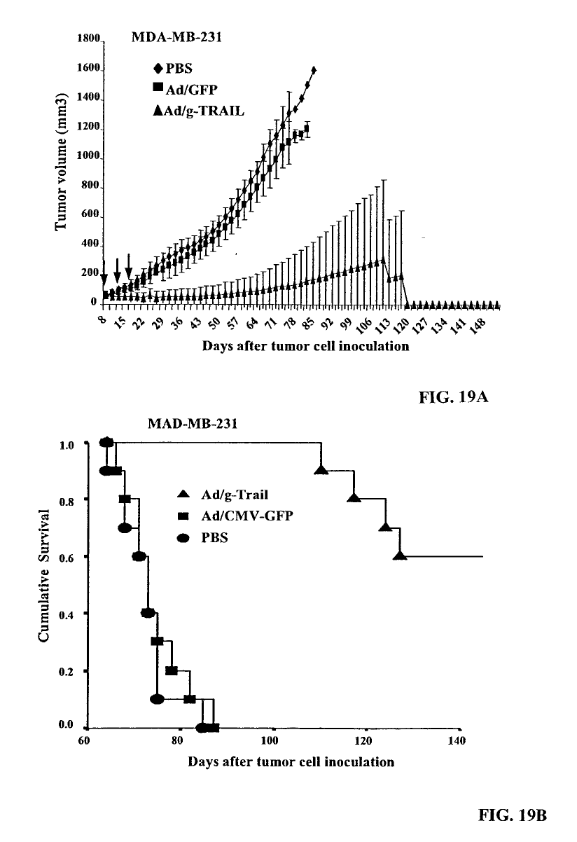

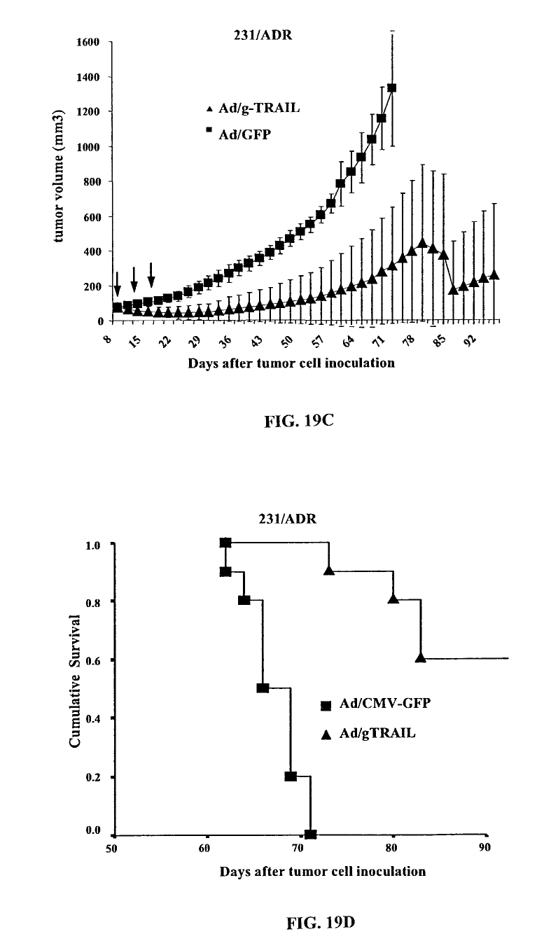

Suppression of tumor growth in vivo. To further evaluate the antitumor activity of Ad/gTRAIL in breast cancer cells, human breast cancer xenografts from MDA-MB-231 were established in 6-8 week old nude mice by inoculating tumor cells subcutaneously. The intralesional administration of Ad/gTRAIL or control vector was initiated when tumors reached 0.4 to 0.5 cm in diameter. Animals received a dose of 9×10¹⁰ particles/injection/tumor every 5 days for a total of three injections. Tumor growth was then monitored. Blood samples were also taken from these animals to test for liver toxicity. The intratumoral injection of Ad/gTRAIL significantly suppressed tumor growth in vivo compared with tumor growth in control groups (P<0.01) (FIG. 19A). Specifically, complete tumor regression occurred in 60% of the animals and these animals remained tumor-free for over 6 months. In comparison, tumors treated with Ad/CMV-GFP grew as fast as those treated with PBS, and all animals in these two groups died within 3 months (FIG. 19B). Histochemical analysis of tumor tissues 2 days after the first injection showed substantial transgene expression in the tumors after treatment with Ad/CMV-GFP or Ad/gTRAIL. However, apoptosis was only observed in the tumors treated with Ad/gTRAIL. Serum liver enzyme assays of aspartate transaminase (AST) and alanine transaminase (ALT) showed all samples were within normal ranges at 2, 12 and 30 days after the first treatment.

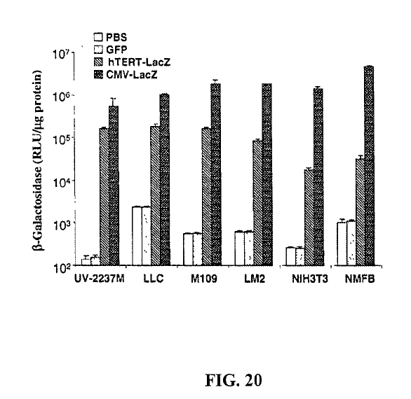

Similar results were observed in mice bearing tumors derived from the doxorubicin-resistant breast cancer cell line 231/ADR. Mice (10/group) bearing 231/ADR tumors were treated with Ad/gTRAIL or Ad/CMV-GFP as described above. Ad/gTRAIL significantly suppressed tumor growth as compared with tumor growth in controls (P<0.05) (FIG. 19C). In addition, four of the ten animals remained tumor-free at 3 months after treatment with Ad/gTRAIL, whereas all the animals in control groups died of tumor burden during this time of period (FIG. 20D).

### EXAMPLE 5

### hTERT Promoter Induces Tumor-Specific Bax Gene Expression and Cell Killing in Syngenic Mouse Tumor Model and Prevents Systemic Toxicity

Materials and Methods

Recombinant adenoviral vectors. Vectors Ad/E1⁻ Ad/hTERT-LacZ, Ad/CMV-LacZ, Ad/GT-LacZ, Ad/GT-Bax, Ad/PGK-GV16, Ad/CMVGFP and Ad/hTERT-GV16 have been previously described. Viral titers were determined as in previous examples. Particle/plaque ratios normally fell between 50:1 and 100:1.

Analysis of in vitro gene expression. Mouse Uv-2237m fibrosarcoma cells were used. Normal mouse fibroblast (NMFB) cells were isolated from the pancreases of Balb/c mice using conventional techniques. Lewis lung carcinoma cells, M109 lung carcinoma cells, LM2 lung epithelial cells, and NIH3T3 cells were maintained in the laboratory. Normal human bone marrow CD34⁺ progenitor cells were separated by magnetic cell sorting (MACS) as described previously (Andreeff et al., 1999). Cells were plated 1 day before vector infection at densities of 1×10⁵/well in 24-well plates. Cells were then infected with adenoviral vectors at multiplicities of infection (MOI) of 3000 viral particles/cell for UV-2237m, LLC, M109 and NMFB cells, 6000 viral particles/cell for NIH3T3 cells, and 10,000 viral particles/cell for LM2 and human CD34⁺ bone marrow stem cells. The optimal MOI of viral infection in each cell line was predetermined by which over 60% of cells were infected. To achieve this high efficiency of adenovirus infection, Superfect was used in combination with adenovirus in LLC, M109, NIH3T3 and CD34⁺ cells as described (Howard et al, 1999). Twenty-four hours after infection, cells were either stained with X-gal to visualize β-galactosidase expression or harvested for biochemical analysis of β-galactosidase activity.

Histochemical studies. X-gal staining was performed as described in example 1. For immunohistochemical analysis of the Bax protein, tumors or livers were fixed in 10% formalin, embedded in paraffin, and then cut into 4-μm sections. To retrieve antigens, the sections were baked, deparaffinized, and heated in citrate buffer (10 mm citric acid, pH 6.0) in a steamer. After endogenous peroxidase was inactivated by a 10-min exposure to 1.5% H₂O₂/methanol, the sections were incubated with blocking serum (goat serum) at room temperature for 30 min, rabbit anti-Bax polyclonal antibody (Santa Cruz Biotechnology, Santa Cruz, Calif., USA) for 1 h, and biotinylated goat anti-rabbit IgG antibody for 30 min. The specific binding of anti-Bax antibody was visualized with an avidin-biotin-peroxidase reagent (Vector Laboratories, Burlingame, Calif., USA) and its substrate diaminobenzidine tetrachloride (Sigma, St Louis, Mo., USA) and by counterstaining with Mayer's hematoxylin.

Assays. β-Galactosidase activities, cell viability assay, and Western analysis for Bax expression were performed as described in previous examples and in (Kagawa et al., 2000). For cell viablity assays, cells were infected with adenoviral vectors at a total MOI of 4500 viral particles/cell. Cells were divided into four groups according to the viral vector system given: Ad/CMVGFP+Ad/PGK-GV16, Ad/GT-Bax+Ad/CMV-GFP, Ad/GT-Bax+Ad/hTERT-GV16, or Ad/GT-Bax+Ad/PGK-GV16. For the apoptosis analysis by flow cytometry, the cells were then infected with recombinant adenoviral vectors at an MOI of 4500 viral particles/cell.

Animal experiments. Animal experiments were performed as discussed in previous examples. For the subcutaneous tumor model, 5×10⁶ UV-2237m cells were inoculated subcutaneously into the dorsal flank of 6- to 8-week-old C3H mice (National Cancer Institute) to establish tumors. The levels of serum aspartate transaminase (AST) and alanine transaminase (ALT) were measured as described (Kagawa et al., 2000).

Results

Transgene expression driven by the hTERT promoter in murine cell lines. hTERT promoter activity was assessed in cultured murine fibrosarcoma UV-2237m cells, Lewis Lung carcinoma (LLC) cells, M109 lung carcinoma cells, LM2 cells, NIH3T3 cells, and normal mouse fibroblast (NMFB) cells by infecting the cells with Ad/hTERT-LacZ as described in Materials and methods. Ad/CMV-LacZ was used as the positive control and Ad/CMV-GFP as the negative control. Expression of bacterial β-galactosidase was analyzed 24 h after infection by either X-gal staining or enzyme assay as described in Materials and methods. In all the tumor cells, both the CMV and hTERT promoters drove strong β-galactosidase expression as shown by Xgal staining, while in NIH3T3 and NMFB cells, only infection with Ad/CMV-LacZ produced high levels of transgene expression. CMV and hTERT promoter activity differed by only three- to 10-fold in tumor cells compared with about 150-fold in NMFB cells (FIG. 20). It is noteworthy that in LM2 cell, a transformed murine lung epithelial cell line derived from a papillary tumor, (Oreffo et al., 1998) CMV and hTERT promoter activity differed by 21-fold; whereas in NIH3T3 cell, a non-transformed special ‘normal’ mouse fibroblastic cell line capable of indefinite growth, CMV and hTERT promoter activity differed by more than 80-fold. These results are in line with those obtained in human cancer cell lines and normal cells (Gu et al., 2000), demonstrating that the hTERT promoter can efficiently use mouse transcription machinery and that hTERT promoter is highly active in murine tumor cells and transformed cells, but relatively quiescent in normal cells.

hTERT promoter-driven Bax gene expression suppresses tumor cells in vitro. A binary adenoviral vector system was recently developed that overcome the difficulties in constructing adenoviral vectors expressing high levels of the strongly apoptotic Bax gene (Kagawa et al., 2000). In brief, the system contains two adenoviral vectors. One of these vectors contains human Bax CDNA under the control of a minimal synthetic promoter comprising five Gal4-binding sites and a TATA box, which is dormant in 293 packaging cells, thus avoiding the toxic effects of the Bax gene on 293 cells and allowing vector (Ad/GT-Bax) production. Expression of the Bax gene can be induced by co-infecting the Ad/GT-Bax virus with another adenoviral vector that expresses a synthetic transactivator consisting of a fusion protein comprising a Gal4 DNA-binding domain and a VP16 activation domain. Administration of this binary vector system to cancer cells elicited extensive apoptosis in vitro and suppressed tumor growth in vivo (Kagawa et al., 2000). The toxicity of the Bax gene in normal cells was prevented by using the hTERT promoter to drive tumor-specific Bax gene expression in human cancer cells (Gu et al., 2000).

To test whether the hTERT promoter can similarly drive Bax gene expression in murine tumor cells, the effects of Bax gene expression induced by the hTERT promoter were compared with that induced by the PGK promoter, a constitutive promoter from mouse housekeeping gene 3-phosphoglycerate kinase. The XTT assay was used to compare cell viability after treating UV-2237m cells, LLC cells and M109 cells with PBS, Ad/CMV-GFP+Ad/PGK-GV16, Ad/GT-Bax+Ad/CMV-GFP, Ad/GT-Bax+Ad/hTERT-GV16, or Ad/GT-Bax+Ad/PGK-GV16 (FIG. 21A). Treatment with either Ad/GT-Bax+Ad/PGK-GV16 or Ad/GT-Bax+Ad/hTERT-GV16 had comparable cell-killing effects on these tumor cells, while all the control treatments had minimal effects on cell viability. The results demonstrate that the hTERT promoter can drive Bax gene expression in murine tumor cells and suppress tumor cell growth in vitro. To confirm that the growth suppression caused by Bax expression was due to apoptosis rather than growth inhibition, fluorescence-activated cell sorter (FACS) analysis was used. UV2237m cells were treated with binary vectors and cells were harvested 48 or 72 h after the treatment and subjected to FACS analysis to determine the fraction of apoptotic cells by quantifying the sub-G1population (FIG. 21B). The Ad/GT-Bax+Ad/hTERT-GV16 and Ad/GT-Bax+Ad/PGK-GV16 treatments resulted in comparable apoptosis populations, suggesting that the hTERT promoter is as strong as the PGK promoter in inducing Bax gene expression and apoptosis in murine tumor cells. Expression of the Bax gene in Uv2237m cells treated by Ad/GT-Bax+Ad/hTERT-GV16 or Ad/GT-Bax+Ad/PGK-GV16 were confirmed by Western analysis. Both hTERT promoter and PGK promoter induced very high expression of Bax gene in Wv-2237m cells, whereas there were minimal Bax expression in control cells.

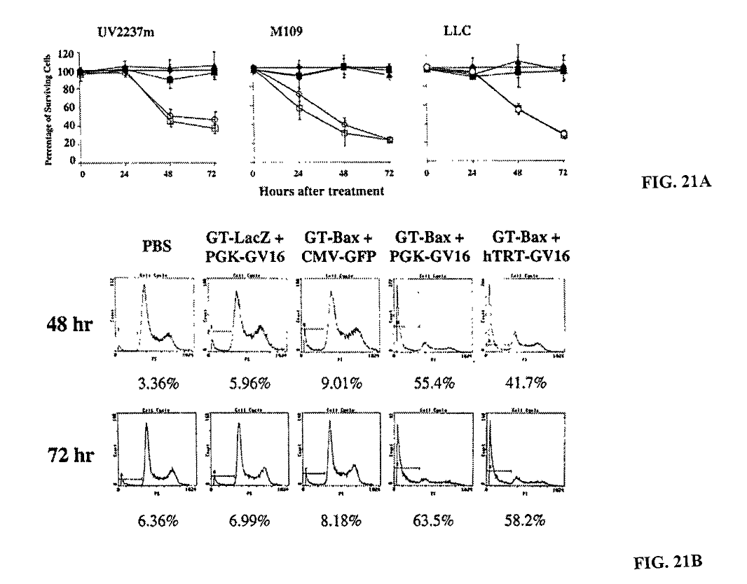

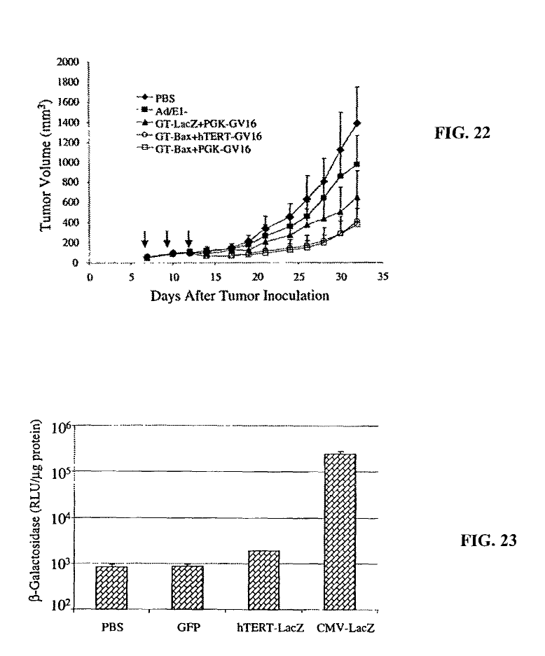

hTERT promoter drives tumor-specific Bax gene expression in vivo and suppresses syngenic tumor growth. To further evaluate the feasibility of using the hTERT promoter for in vivo Bax gene therapy in syngenic tumors, UV-2237m tumors were established subcutaneously in immune-competent C3H mice and treated the tumors with hTERT or PGK promoter-driven Bax gene vectors. After three sequential intratumoral injections of the vectors, tumor size changes were monitored for 3 weeks, by which time the mice in control groups had to be killed according to institutional policy because the tumor sizes reached 15 mm in diameter. Treatment with Ad/GT-Bax+Ad/hTERT-GV16 or Ad/GT-Bax+Ad/PGK-GV16 resulted in the same level of tumor growth suppression, which was significantly different from the changes resulting from treatments with PBS, Ad/E1⁻, or Ad/GT-LacZ+Ad/PGK-GV16 (FIG. 22). The group treated with Ad/GT-LacZ+Ad/PGK-GV16 also showed mild inhibition of tumor growth, probably due to the immune response in C3H mice (Lu et al., 1999). Expression of the Bax gene in tumors was confirmed by immunohistochemical staining. Intratumoral injection of either Ad/GT-Bax+Ad/hTERT-GV16 or Ad/GT-Bax+Ad/PGK-GV16 binary vector resulted in strong Bax gene expression in Uv-2237m tumors. In comparison, when the binary vectors were systemically injected through the tail veins of mice, only the PGK promoter induced strong Bax expression in the liver, while the hTERT promoter did not induce detectable Bax expression. These results demonstrate that the hTERT promoter is highly active in murine tumors, but quiescent in normal liver in vivo and that it effectively drives tumor-specific Bax gene expression in vivo and suppresses syngenic tumor growth.

hTERT promoter prevents acute liver toxicity of Bax gene with no obvious long-term toxicity. To test the potential toxicity of systemic delivery of the Bax gene in mice, adult BALB/c mice in groups of 10 were infused via the tail vein with PBS, Ad/GT-Bax+Ad/CMV-GFP, Ad/GT-Bax+Ad/hTERT-GV16, or Ad/GT-Bax+Ad&PGK-GV16 at a dose of 6×10¹⁰ viral particles/mouse, three times within 1 week. The mice were monitored for up to 6 months. Blood samples were collected at 2 days, 10 days, 30 days, 100 days and 6 months after the last injection to determine the hemogram and serum levels of AST and ALT. The most significant toxic effects occurred in the group treated with Ad/GT-Bax+Ad/PGK-GV16: within 1 week of viral injections, six out of the 10 mice died of acute liver toxicity (P≦0.01). The remaining four in the group recovered and survived the 6-month experiment. In contrast, none of the mice in the Ad/GT-Bax+Ad/hTERT-GV16 group or the other control groups died during the experiment. In all of the mice that finished the experiment, none had significant differences in hemoglobin level or white blood cell (WBC) or red blood cell (RBC) counts, suggesting that the bone marrow toxicity of the adenovirus mediated Bax gene is minimal (Table 6). As reported previously (Gu et al., 2000), AST and ALT levels were significantly higher (P≦0.001) only in the Ad/GT-Bax+Ad/PGKGV16 group 2 days after viral injection than in all other groups, indicating acute liver toxicity caused by Bax expression. From day 10 and thereafter, no significant differences in AST or ALT were seen in any of the groups (Table 7). Together, these results suggest that the hTERT promoter can be used to prevent the acute liver toxicity of proapoptotic genes systemically while having no obvious long-term toxic effects.

TABLE 6
Short- and long-term effects of adenovirus-mediated Bax expression on
blood cells.

| | | | | GT-Bax | |
|---|---|---|---|---|---|
| | | | GT-LacZ | hTERT- | GT-Bax |
| | Days | PBS | PGK-GV16 | GV16 | PGK-GV16^{α} |
| Hg | 2 | 12.80 ± 0.57 | 12.60 ± 0.00 | 11.93 ± 0.21 | 11.36 ± 1.03 |
| (g/dl) | 10 | 14.20 ± 0.87 | 12.60 ± 0.46 | 13.37 ± 0.35 | 13.93 ± 0.70 |
| | 30 | 13.13 ± 1.50 | 13.20 ± 0.96 | 12.90 ± 0.59 | 14.15 ± 0.42 |
| | 100 | 13.45 ± 1.00 | 12.73 ± 0.74 | 12.73 ± 0.63 | 12.85 ± 0.62 |
| | 180 | 13.42 ± 0.77 | 12.60 ± 0.56 | 12.80 ± 0.64 | 12.83 ± 0.94 |
| WBC | 2 | 3.43 ± 1.32 | 4.21 ± 0.04 | 3.66 ± 0.65 | 4.46 ± 2.06 |
| (10³/μl) | 10 | 5.08 ± 2.17 | 3.95 ± 0.40 | 5.13 ± 0.97 | 5.89 ± 2.50 |
| | 30 | 5.03 ± 1.37 | 5.38 ± 0.37 | 4.02 ± 0.56 | 6.46 ± 2.37 |
| | 100 | 10.47 ± 2.01 | 7.38 ± 2.62 | 8.25 ± 3.30 | 6.71 ± 1.22 |
| | 180 | 7.37 ± 1.98 | 6.32 ± 2.65 | 5.74 ± 2.93 | 6.13 ± 1.73 |
| RBC | 2 | 8.67 ± 0.10 | 8.91 ± 0.29 | 7.73 ± 0.37 | 8.36 ± 0.9 |
| (10³/μl) | 10 | 9.66 ± 0.58 | 8.99 ± 0.55 | 9.27 ± 0.25 | 9.15 ± 0.93 |
| | 30 | 10.07 ± 0.77 | 9.61 ± 0.58 | 9.28 ± 0.30 | 9.68 ± 0.62 |
| | 100 | 10.10 ± 0.91 | 8.84 ± 0.37 | 9.02 ± 0.34 | 8.78 ± 0.19 |
| | 180 | 9.29 ± 1.20 | 8.58 ± 0.51 | 8.75 ± 0.34 | 9.09 ± 0.33 |

Values are mean ± s.d. for three to five samples.
^{α}Six mice died within 1 week after adenovirus injection.

TABLE 7
Short- and long-term effects of adenovirus-mediated Bax expression on
serum levels of liver enzymes.

| | | | GT-LacZ + | GT-Bax + | GT-Bax + |
|---|---|---|---|---|---|
| | Days | PBS | PGK-GV16 | hTERT-GV16 | PGK-GV16 |
| ASL | 2 | 28 ± 3 | 25 ± 5 | 27 ± 10 | 475 ± 201^{a} |
| (IU/l) | 10 | 58 ± 15 | 47 ± 8 | 57 ± 20 | 58 ± 23 |
| | 30 | 106 ± 67 | 102 ± 14 | 89 ± 19 | 120 ± 34 |
| | 100 | 104 ± 54 | 102 ± 18 | 92 ± 30 | 88 ± 34 |
| | 180 | 99 ± 43 | 118 ± 21 | 105 ± 33 | 80 ± 31 |
| ALT | 2 | 85 ± 9 | 87 ± 8 | 50 ± 9 | 770 ± 405a |
| (IU/l) | 10 | | | | |
| | 30 | 54 ± 8 | 48 ± 12 | 42 ± 18 | 50 ± 11 |
| | 100 | 46 ± 5 | 47 ± 8 | 29 ± 11 | 59 ± 54 |
| | 180 | 40 ± 10 | 62 ± 8 | 59 ± 16 | 36 ± 5 |

Values are mean ± s.d. for three to five samples.
^{a}P ≦ 0.001.

Minimal hTERT activity in hematopoietic CD34⁺ progenitor cells. One of the major concerns about the use of the hTERT promoter to drive expression of proapoptotic or cytotoxic genes is its potential toxicity to stem cells. To test whether hTERT is active in progenitor stem cells, normal human bone marrow CD34⁺ hematopoietic progenitor cells were isolated and the β-galactosidase activity of these cells compared when infected with Ad/hTERT-LacZ or Ad/CMV-LacZ. Adenoviral vectors infect stem cells poorly. A very high dose of an adenoviral vector and prolonged cell-vector contact are required to infect stem cells, and even under these conditions, only a limited percentage of stem cells will be infected (Watanabe et al., 1996; Feldman et al., 1997). However, many liposome reagents, such as Superfect (Qiagen, Hilden, Germany), have been shown to increase the efficiency of adenoviral infection of stem cells (Howard et al., 1999). Indeed, when Superfect was combined with Ad/CMV-GFP, the fluorescent (i.e. infected) population of CD34⁺ cells reached 60% at MOI of 10,000 (data not shown). When human CD34⁺ progenitor cells were infected with Ad/hTERTLacZ or Ad/CMV-LacZ under similar conditions, the difference in β-galactosidase activity was more than 100-fold (FIG. 23), which is similar to that in other normal cells (FIG. 20) (Gu et al., 2000). Moreover, hTERT promoter activity was very close to basal levels, indicating hTERT promoter activity is very low in these CD34⁺ progenitor cells. The low transcriptional activity of the hTERT promoter in human progenitor cells and the lack of detectable changes in blood cell profiles in the long-term in vivo study suggest that the potential stem cell-related toxicity of adenovirus mediated, hTERT-driven proapoptotic gene expession, if any would be limited.

### EXAMPLE 6

### Overcoming Resistance to Adenovirus-Mediated Proapoptotic Gene Therapy in DLD1 Human Colon Cancer Cell Line

Materials and Methods

Cell lines and adenoviruses. Cells of the human colon cancer cell line DLD1 were grown in RPMI 1640 supplemented with 10% fetal bovine serum (FBS) and antibiotics. DLD1 cells stably transfected with the human Bcl-xL gene were obtained by transfecting DLD1 cells with pGT60-hBcl-xL (InvivoGen, San Diego, Calif., USA) using LipofectAMINE (Invitrogen, Carlsbad, Calif., USA). Cells were then cultured in RPMI 1640 medium containing 500 μg/ml hygromycin. Hygromycin-resistant single-cell clones overexpressing Bcl-xL were picked up and identified by Western blot analysis. The adenoviral vectors used, Ad/PGK-GV16, Ad/hTERT-GV16, Ad/GT-Bak, Ad/GT-Bax, Ad/GT- TRAIL, and Ad/CMV-GFP, were described previously (Gu et al., 2000; Kagawa et al., 2001). Ad/gTRAIL, was constructed as previously described. Both expression cassettes are inserted into the adenoviral E1 region, in a right-to-left sequence order direction, as GT-GFP/TRAIL-simian virus 40 polyadenylation signal-hTERT-GAL4/VP16 bovine growth hormone polyadenylation signal. The GAL4 gene regulatory components are included in the vector Ad/gTRAIL because recent study showed that the yeast GAL4 gene regulatory system can augment transgene expression from a tumor-specific promoter without compromising promoter-specificity (Koch et al., 2001). The expansion, purification, titration, and quality analysis of all the vectors used were performed as previously described (Kagawa et al., 2000; Gu et al., 2000; Kagawa et al., 2001).

In vitro gene transfer, cell viability and flow cytometry assays. In vitro gene transfer was conducted as previously described using DLD1 cells. Cell viability assays were conducted as described in previous examples using cells seeded on 96-well plates at densities of 1×10⁴ cells/well 1 day before infection. Flow cytometry analysis for GFP expression and apoptosis were performed as previously described.

Western blot analysis. Rabbit anti-human DR4, and mouse monoclonal antibodies against human Bax, Bak, XIAP, caspase-2, -7, and 8 were purchased from PharMingen (San Diego, Calif., USA). Rabbit anti-human DR5 was obtained from Imegenex (San Diego, Calif., USA). The rabbit antihuman/ mouse FLIP was provided by R&D Systems Minneapolis, Minn., USA). The rabbit anti-human Bcl-2 and Bcl-xL/S were purchased from Santa Cruz Biotechnology (Santa Cruz, Calif., USA). For Western blot analysis, 80 μg of total cellular proteins was separated by 10-12% sodium dodecyl sulfate-polyacrylamide gel electrophoresis (SDS-PAGE) and then transferred to Hybond enhanced chemiluminescence membranes (Amersham, Arlington Heights, Ill., USA). Membranes were then blocked with blocking buffer containing 5% low-fat milk and 0.05% Tween-20 in PBS for 1 h or overnight at 4° C., washed three times with PBS containing 0.05% Tween-20 PBST), and then incubated with primary antibodies for at least 1 h at room temperature. After washing with PBST again, membranes were incubated with peroxidase conjugated secondary antibodies and developed with a chemiluminescence detection kit (ECL kit, Amersham Phamarcia).

RNase protection assays. The total RNA was extracted using a total RNA isolation kit (PharMingen). RNase protection assay was performed according to the manufacturer's protocol (RiboQuant Multiprobe RNase Protection Assay System, PharMingen). Human glyceraldehyde-3-phosphate dehydrogenase (GAPDH) was used as an internal control for normalization of the mRNA amount.

Real-time polymerase chain reaction. Real-time polymerase chain reaction (PCR) analysis was performed in the ABI Prism 7700 Sequence Detection System according to the protocol of the manufacturer Applied Biosystems, Foster City, Calif., USA). Primers and probes for each gene tested were designed with built-in software in the 7700 System provided by Applied Biosystems and sysnthesized by Invitrogen (Frederick, Md., USA). Primers were placed in different exons of a gene eliminate the effect from contaminated genomic DNA. For example, the forward primer for Bcl-xL was 5′- GTCGGATCGCAGCTTGGA-3′, located in exon 2, and the reverse primer was 5′-GCTGCTGCATTGAACCCAT- AGAGTTC-3′, located in exon 3. The probe sequence for Bcl-xL was 5′-GTCGGATCGCAGCTTGGA-3′, located in exon 2. All probes used were labeled at the 5′-end with carboxy-fluorescein phosphoramidite as a reporter dye and at the 3′-end with carboxy-tetramethyl-rhodamine for quenching. One microgram of total RNA from each cell line was reverse-transcribed into cDNA by using random hexamers as reverse transcription primer (TaqMan Reverse Transcription Reagents, Applied Biosystems). The human GAPDH gene was used as an internal control for normalization of the mRNA amount. A typical real-time PCR mix (25 μl ) contained the sample DNA (or cDNA), 10× TaqMan Buffer (2.5 μl), 200 μM dATP, dCTP, dGTP, and 400 μM dUTP, 5 mM MgCl₂, 0.65 units of AmpliTaq Gold, 0.25 units of AmpErase uracil N-glycosylase (UNG), 200 nM each primer, and 100 nM probe. The thermal cycling conditions consisted of one cycle at min for 50° C. and 10 min for 95° C., and 50 cycles of 95° C. for 15 s and 60° C. for 1 min. All reactions were performed duplicate. After the reaction, the built-in software of the 7700 System was used to analyze all the data and to generate the standard curve. The Ct (threshold cycle) value of each testing sample and its corresponding starting quantity were based on the standard curve. Statistical analysis was performed as prevously described.

Results

Selection of DLD1/Bax-R and DLD1/TRAIL-R cells. To determine whether resistance develops during adenovirus-mediated proapoptotic gene therapy, the human colon cancer line DLD1 was treated with binary adenoviral vectors that expressed either Bax (Ad/PGK-GV16+Ad/GT-Bax) or TRAIL (Ad/PGK-GV16+Ad/GT-TRAIL) at a total MOI of 1000 vp/cell (Kagawa et al., 2001; Kagawa et al., 2000). Seventy-two hours after infection, about 95% of cells in each treatment were apoptotic. In contrast, treatment with a control vector Ad/CMV-GFP at the same total dose resulted in only background levels of cell death that were similar to the level of apoptosis seen in mock control. These results demonstrate that parental DLD1 cells were susceptible to both Bax and TRAIL gene delivered by adenoviral vectors. After treatment with the Bax- or TRAIL-expressing adenoviral vectors, the surviving DLD1 cells were allowed to grow in regular medium. When these cells reached 70-80% confluency, a second round of infection at the same MOI was performed. Seventy-two hours after the fourth round of infection of surviving cells with either the Bax- or the TRAIL-expressing adenoviral vectors, the level of cell death in each treatment was less than 5%, suggesting that these cells were resistant to the respective gene Bax or TRAIL driven by-PGK promoter and delivered by adenoviral vectors. These two resistant cell lines were named DLD1/Bax-R and DLD1/TRAIL-R cells.

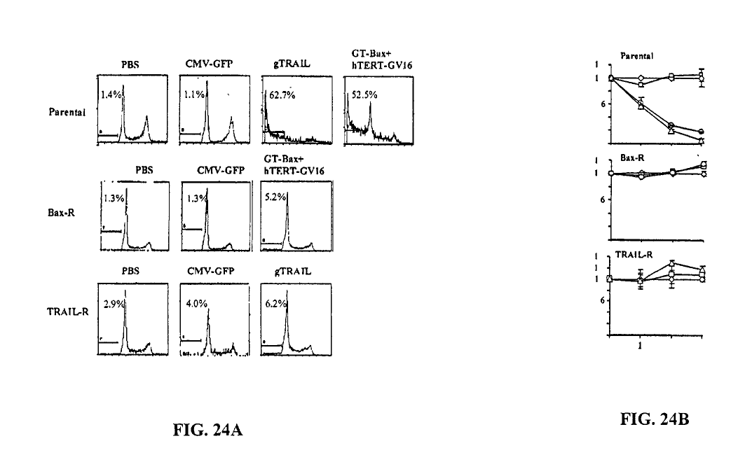

Whether these two resistant cell lines were also resistant to the Bax or the TRAIL gene driven by hTERT promoter (Gu et al., 2000) and delivered by adenoviral vectors was tested. Parental DLD1, DLD1/Bax-R and DLD1/TRAIL-R cells were treated with Ad/hTERT-GV16+Ad/GT-Bax or with bicistronic adenoviral vector Ad/gTRAIL at a total MOI of 1000 vp/cell. PBS was used as a mock control and Ad/CMV-GFP as a vector control. In all three cell lines tested, FACS analysis showed that only background levels of cells death (0.4-2.1%) 48 h after treatment with either PBS or Ad/CMV-GFP. In parental DLD1 cells, FACS analysis showed that the percentage of apoptotic (sub-G1) cells was 52.5% 48 h after treatment with Ad/hTERT-GV16+Ad/GT-Bax and 62.7% 48 h after treatment with Ad/gTRAIL. In contrast, only 5.2% of DLD1/Bax-R cells were apoptotic 48 h after treatment with Ad/hTERT-GV16+Ad/GT-Bax, and only 6.2% of DLD1/TRAIL-R cells were apoptotic 48 h after treatment with Ad/gTRAIL (FIG. 24A). These results demonstrated that the mechanisms of resistance of DLD1/Bax-R and DLD1/TRAIL-R cells do not involve the PGK promoter. Cell viability assay with XTT showed the same results (FIG. 24B) as those of the FACS assay. Almost all parental DLD1 cells treated with Ad/hTERT-GV16+Ad/GT-Bax or with Ad/gTRAIL were apoptotic 3 days after infection. In contrast, there was no significant difference in the cell death levels between DLD1I/Bax-R cells treated with Ad/hTERT-GV16+Ad/GT-Bax and the control vectors or between DLD1/TRAIL-R cells treated with Ad/gTRAIL and the control vectors.

Susceptibility of DLD1/Bax-R and DLD1/TRAIL-R cells adenoviral infection. To determine whether resistance is caused by reduced adenoviral vector transduction, the transduction efficiencies of adenoviral vectors in parental DLD1, DLD1/Bax-R, and DLD1/TRAIL-R cells was evaluated. Cells were infected with either Ad/CMV-GFP or Ad/gTRAIL a total MOI of 1000 vp/cell. Cells were harvested 48 h after infection and subjected to FACS analysis. Treatment with Ad/CMV-GFP resulted in levels of GFP-positive cells of 98±2.0% for parental DLD1 cells (mean fluorescence intensity 45±4.3), and 70±4.5% for DLD1/Bax-cells (mean fluorescence intensity 4±1.2), and 99±1.5% for DLD1/TRAIL-R cells (mean fluorescence intensity 42±3.9). Thus the level of transgene expression of DLD1/TRAIL-R cells is similar to that of the parental DLD1 cells, in terms of both the percentage and the mean fluorescence intensity of GFP-positive cells. However, in DLD1/Bax-R cells, the level of transgene expression was dramatically reduced. Similar results were obtained when the three cell lines were treated with Ad/gTRAIL. Because Ad/gTRAIL expresses the GFP/TRAIL fusion protein from the hTERT promoter, analysis of GFP-positive cells allowed us to estimate levels of transgene expression after treatment with Ad/gTRAIL. Levels of transgene expression were similar in parental DLD1 and DLD1/TRAIL-R cells, but dramatically reduced in DLD1/Bax-R cells (Table 8). These observations were consistent with the results of Western blot analysis. Compared with parental DLD1 cells, DLD1/Bax-R cells expressed much less Bax protein after treatment with Ad/hTERT-GV16+Ad/GT-Bax at a total MOI of 1000 vp/cell. Increasing the total vector dose to five times the original dose (Ad/hTERT-GV16+Ad/GT-Bax at a total MOI of 5000 vp/cell) still did not induce Bax-R cells to express levels of Bax protein similar to the level expressed by parental DLD1 cells treated with an MOI of 1000 vp/cell.

TABLE 8
Susceptibility of parental, Bax-R and TRAIL-R DLD1 cells to
adenoviral infection.

| | CMV-GFP | | gTRAIL | |
|---|---|---|---|---|
| | Positive | | Positive | |
| Cells | cells (%) | Mean intensity | cells (%) | Mean intensity |
| Parental | 98 ± 2.0 | 45 ± 3 | 80 ± 3.2 | 30 ± 1.3 |
| Bax-R | 70 ± 4.5* | 4 ± 1.2** | 17 ± 3.2** | 3 ± 1.2** |
| TRAIL- | 99 ± 1.5 | 42 ± 3.9 | 84 ± 1.2 | 25 ± 2.3 |
| R | | | | |

Values are means ± s.d. for a quadruplicate assay,
*P < 0.05,
**P < 0.01 compared with parental DLD1.

To further investigate the differences in susceptibility adenoviral infection in parental DLD1 and DLD1I/Bax- cells, these two cell lines were infected with Ad/CMV-GFP at different MOIs. The percentage of GFP-positive cells and the mean fluorescence intensity were then determined by FACS analysis as described above. DLD1/Bax-R cells treated with an MOI of 10,000 vp/cell had levels of GFP expression equivalent to those of parental DLD1 cells treated with an MOI of 1000 vp/cell. Together, these results suggested that resistance to adenoviral infection may be responsible for resistance in DLD1/Bax-R cells.

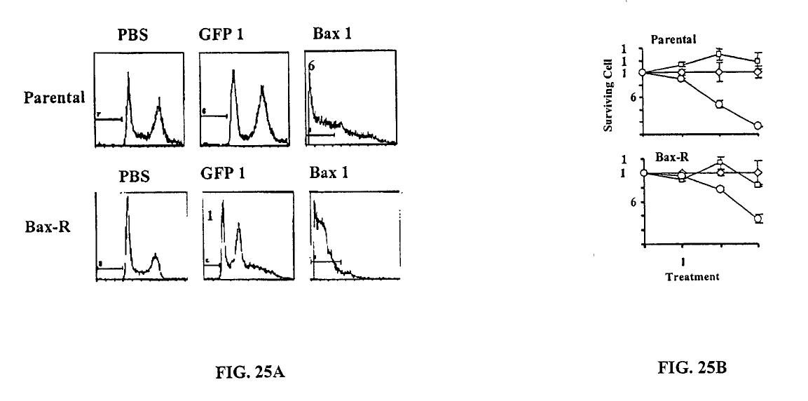

Cell killing effects by dose escalation. To further investigate whether reduced transduction efficiency is sufficient to induce the resistance in DLD1/Bax-R cells, DLD1/Bax-R cells were infected with Ad/hTERT-GV16+Ad/GT-Bax at a total MOI of 10,000 vp/cell. Cells infected with Ad/CMV-GFP at the same total MOIs were used as controls. Levels of apoptosis were then determined by FACS analysis and compared with the levels of apoptosis among parental DLD1 cells treated with Ad/hTERT-GV16+Ad/GT-Bax at a total MOI of 1000 vp/cell (FIG. 25A). The level of apoptosis for DLD1/Bax-R cells treated with Ad/hTERT-GV16+Ad/GT-Bax at a total MOI of 10,000 vp/cell was similar to that for parental DLD1 cells treated with the same vectors at a total MOI of 1000 vp/cell. DLD1/Bax-R cells treated with control vector at an MOI of 10 000 vp/cell showed only a background level of cell death similar to that of mock controls. This observation was further supported by cell viability assay with XTT (FIG. 25B). A vector dose that resulted in transgene expression equivalent to that in parental DLD1 cells resulted in significant cell killing in DLD1/Bax-R cells, suggesting that reduced transduction efficiency may account for resistance in DLD1/Bax-R cells.

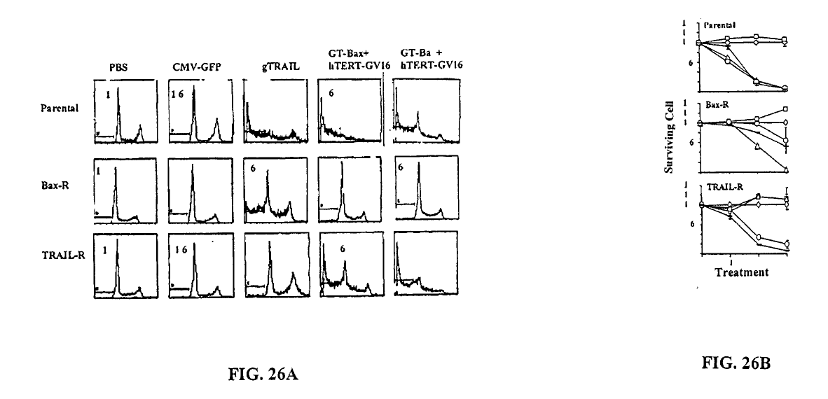

Susceptibility to adenoviral vectors expressing alternative proapoptotic genes. Whether DLD1/Bax-R or DLD1/TRAIL-R cells were susceptible to adenoviral vectors that expressed alternative proapoptotic genes without dose escalation was investigated. For this purpose, groups of parental DLD1, DLD1/Bax-R, and DLD1/TRAIL-R cells were treated with Ad/hTERT-GV16+Ad/GT-Bax, Ad/hTERT-GV16+Ad/GT-Bak, or Ad/gTRAIL at a total MOI of 1000 vp/cell. Apoptotic cell death was quantified by FACS assay. Parental DLD1 cells were susceptible to all the three treatments, whereas DLD1/Bax-R cells were resistant to adenoviral vectors expressing either the Bax or the Bak gene, but were susceptible to the adenoviral vector expressing the TRAIL gene. At 48 h after treatment with Ad/gTRAIL, about 36.4% of DLD1/Bax-R cells were apoptotic. In contrast, only background levels (<6.5%) of DLD1/Bax-R cells were apoptotic after treatment with adenoviral vectors expressing either the Bax or the Bak gene. DLD1/TRAIL-R cells remained resistant to Ad/gTRAIL, but were susceptible to adenoviral vectors expressing either the Bax or the Bak gene. For DLD1/TRAIL-R cells, the level of apoptosis was 45.6% 48 h after treatment with Ad/hTERT-GV16+Ad/GT-Bax and 54.3% at 48 h after treatment with Ad/hTERT-GV16+Ad/GT-Bak (FIG. 26A). Cell viability assay with XTT showed similar results (FIG. 26B). Almost all parental DLD1 cells treated with Ad/hTERT-GV16+Ad/GT-Bax, Ad/hTERT-GV16+Ad/GT-Bak, or Ad/gTRAIL were apoptotic at 3 days after infection. In contrast, no significant difference was observed between groups of DLD1/Bax-R cells treated with Ad/hTERT-GV16+Ad/GT-Bax, Ad/hTERT-GV16+Ad/GT-Bak, or control vectors, but almost all DLD1/Bax-R cells treated with Ad/gTRAIL were killed. For DLD1/TRAIL-R cells, no difference in cell viability levels was observed between cells treated with Ad/gTRAIL and cells treated with control vector, but more than 80% of cells treated with Ad/hTERT-GV16+Ad/GT-Bax or Ad/hTERT-GV16+Ad/GT-Bak were killed. Together, these results suggested that DLD1/Bax-R and DLD1/TRAIL-R cells are susceptible to adenoviral vectors expressing proapoptotic genes involved in different apoptotic pathways or different models of cell killing, even without escalation of vector doses.

Molecular difference in parental DLD1, DLD1/Bax-R and DLD1/TRAIL-R cells. To investigate molecular differences among these three cells, the levels of several proteins that are known to be involved in Bax- or TRAIL-mediated apoptosis pathways, including Bcl2; Bcl-xL; Bcl-xS; DR4; DR5; DcR1; FLIP; Bax; Bak; XIAP and caspase-2, -7, -8, and -9 were analyzed. RNase protection assay and real-time PCR analysis were both used to determine mRNA levels. Western blot analysis was used to determine protein levels. Human GAPDH was used as an internal control for mRNAs. β-Actin was used as load control for Western blot analysis. In most cases, mRNA levels determined by RNase protection assay or real-time PCR assay correlated well with protein levels determined by Western blot analysis. However, there were some discrepancies. For example, the RNase protection assay showed comparable levels of DR4 expression between DLD1 and DLD1/TRAIL-R cells and a much lower level in DLD1/Bax-R cells. In contrast, Western blot analysis showed comparable DR4 levels between DLD1 and DLD1/Bax-R cells, but a slightly lower level in DLD1/TRAIL-R cells. In these cases, values that were consistent between the two methods of assay were considered true values. No dramatic differences were found for caspase-2, -7, -8, or -9; DR5; DcR1; Bcl-xS; Bcl-2; Bax; Bak; FLIP or XIAP among the parental DLD1, DLD1/Bax-R and DLD1/TRAIL-R cells (data not shown). However, Bcl-xL was up-regulated in DLD1/TRAIL-R cells with both RNA (real-time PCR) and protein levels about three times higher than those in either parental DLD1 or DLD1/Bax-R cells.

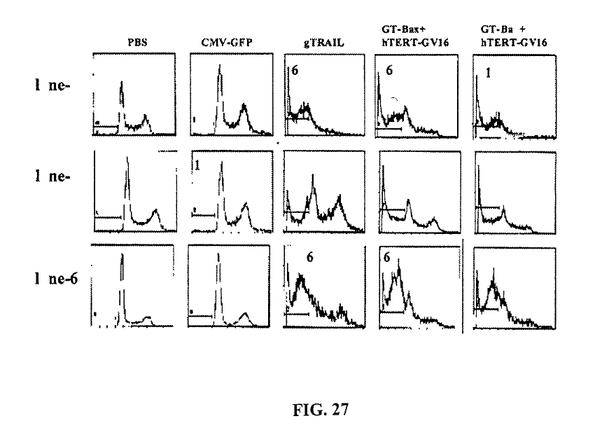

Bcl-xL overexpression does not protect DLD1 cells fromTRAIL-, Bax-, or Bak-induced apoptosis. Because Bcl-xL was the only gene overexpressed as shown both by RNA levels and by protein levels in DLD1/TRAIL cells, whether overexpression of the Bcl-xL gene is responsible for resistance in DLD1/TRAIL-R cells was tested. DLD1 cells overexpressing Bcl-xL were then constructed by transfection with the plasmid pGT60-hBcl-xL and selected against hygromycin. Of seven hygromycin-resistant clones tested for Bcl-xL expression by Western blots, five overexpressed the Bcl-xL gene at different levels. The level of apoptosis induced in each clone by treatment with adenoviral vectors expressing the Bax, Bak, or TRAIL gene was then measured. Regardless of the level of Bcl-xL expression, all five clones tested were susceptible to treatment with Ad/hTERT-GV16+Ad/GT-Bax, Ad/hTERT-GV16+Ad/GT-Bak, or Ad/gTRAIL. At 48 h after treatment, the percentage of apoptotic cells in sub-G1 phase ranged from 30.0% to 71.4% (FIG. 27). These results suggested that Bcl-xL overexpression does not protect DLD1 cells from adenoviral vectors expressing the TRAIL, Bax, or Bak gene.

### EXAMPLE 7

### A novel Single Tetracycline-Regulative Adenoviral Vector for Tumor-Specific Bax Gene Expression and Cell Killing in vitro and in vivo

Materials and Methods

Construction of recombinant adenovirus vectors. Vectors Ad/GT-Bax and Ad/hTERT-GV16 were constructed as described previously (Gu et al., 2000). Ad/CMV-GFP was provided by Dr. T. J. Liu (MDACC, Houston, Tex.). Ad/gBax was constructed using a previously described method (Kagawa et al., 2000). Briefly, an adenoviral shuttle vector (pAd/hTERT-gBax) was constructed that contained two expression cassettes, one for the GFP-Bax fusion, whose gene is driven by a synthetic minimal promoter composed of tetracycline-responsive elements (TRE) and CMV mini- promoter, and the other for rTA, a transactivator whose gene is driven by the hTERT promoter. This shuttle plasmid was then cotransfected into 293 cells along with a 35-kb Clal fragment from adenovirus type 5 in the presence of 10 mg/ml tetracycline (Tc). Recombinant vector Ad/gBax was generated by homologous recombination and plaque-purified. The expansion, purification, titration, and quality analysis of all vectors used were performed as described previously. The titer and yield for Ad/gBax were in the range seen for other E1 -deleted adenoviral vectors when amplified n the presence of tetracycline.

Cell lines and in vitro gene transfer. Human lung cancer cell lines H1299, A549, H358, and H322, hepatoma cancer cell line HepG2, cervix cancer cell line Siha, ovarian cancer cell line OVCAR3, prostate cancer cell line DU145, bladder cell line HTB9 and osteosarcoma cell line Saos-2 were originally obtained from the American Type Culture Collection (ATCC). Colon cancer cell line Lovo was obtained from Dr. T. Fujiwara (Okayama University, Japan). Normal human lung fibroblast (NHFB) cells were purchased from Clonetics (San Diego, Calif., USA). The optimal MOI, at which over 80% of the cells were infected by reporter virus as determined by pilot experiments, for these cell lines were: 2000 viral particles/cell for H1299, H358, Lovo and HepG2 cells and 3000 viral particles/cell for A549, H322, DU145, OVCAR, HTB9 and NHFB cells. For fluorescence and cell morphology experiments, cells were plated onto 60 mm dishes at a density of 2×5⁵/dish and treated with viruses and 48 h later, pictures were taken with a fluorescence microscope for GFP expression or a Nikon digital camera for cell morphology. The XTT assay was performed as previously described.

Apoptosis assays and Animal experiments. Flow cytometry and TUNEL staining assays were conducted as previously described. For flow cytometry assays cells were plated at densities of 1×10⁶/100 mm and infected with different recombinant adenoviral vectors.

Animal experiments were performed as previously described, except that in the subcutaneous tumor model, 5×10⁶ H1299 cells were inoculated subcutaneously into the dorsal flank of 6- to 8-week-old nude mice (Harlan-Sprague Dawley, Indianapolis, Ind., USA) to establish tumors. Statistical analysis was conducted as previously described.

Results

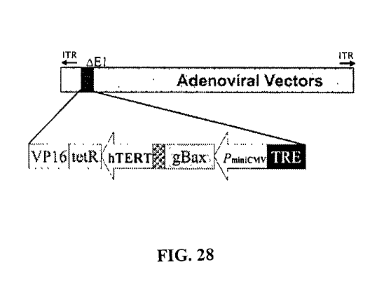

Construction of Ad/gBax. The high activity of the hTERT promoter in 293 cells and the strong pro-apoptotic activity of Bax protein led to the design and construct of a bicistronic adenoviral vector, Ad/hTERT-gBax. This bicistronic vector as modified combined the two expression cassettes of the Tet-Off system (BD Clontech, San Francisco, Calif., USA) into a single vector (FIG. 28). One expression cassette consisted of a transactivator, tTA, which is a fusion of the tetracycline repressor (TetR) to the VP16 activation domain and is driven by the hTERT promoter. The second cassette contained the cDNA of the GFP/Bax fusion protein under the control of a compound promoter consisting of TRE and the minimal immediate early promoter of CMV. GFP was included to facilitate the selection of recombinant virus and the detection of expression products. In the absence of Tc, hTERT would drive the expression of tTA protein, which would then turn on the expression of GFP-Bax. However, when Tc is added, for instance, in 293 cells, it will bind to the tTA protein and inhibit the binding of tTA to TRE, thus inhibiting the expression of GFP-Bax and protecting the 293 cells from Bax-induced apoptosis, hence allowing virus particle packaging and propagation. Using the above strategy, Ad/gBax was successfully constructed and the titers of Ad/gBax produced in the presence of tetracycline were in line with those of control, nontoxic vectors (data not shown). The GFP-Bax expression induced by Ad/gBax after a 24-h infection in H1299 lung cancer cells, and the repressibility of gene expression by Tc was observed. As little of 1 ng/ml Tc started to inhibit GFP-Bax expression; 5 ng/ml significantly reduced the GFP-Bax level; and by 1 mg/ ml, GFP-Bax was expressed at a level close to endogenous Bax protein (data not shown). Further increases in the Tc concentration, however, did not inhibit GFP-Bax anymore (data not shown), suggesting the bottom-line leakage of the Tet-Off system.

Ad/gBax drives tumor-specific GFP/Bax expression in human cancer cells. To test whether Ad/gBax can drive tumor-specific GFP-Bax gene expression and induce apoptosis, several human cancer cell lines from varying origins were used and the expression of GFP-Bax from Ad/hTERT-gBax with the expression of GFP from Ad/CMV-GFP. These cancer cell lines include: lung cancers with various p53 status H1299 (null), H358 null H322 (mut), and A549 (wt); HepG2 (liver); Lovo (colon); Siha (cervix); OVCAR3 (ovary); DU145 (prostate); HTB9 (bladder) and Saos-2 (bone). NHFB cells were used as a control. At 48 h after virus infection, Ad/CMV-GFP prove very strong GFP expression in all tumor and normal cells, while Ad/gBax only induced a high level of GFP-Bax in cancer cells. There was no detectable GFP-Bax in the NHFB cells. More importantly, many of the cells of cancer cell lines underwent apoptosis, suggesting that the fusion protein GFP-Bax has similarly strong pro-apoptotic activity to that of the intact Bax protein. These cancer anrd normal cells have varying endogenous Bax protein expression (data not shown), which apparently is not a factor in determining the apoptotic effect of Ad/gBax. It is noteworthy that Ad/gBax induced weak GF-Bax expression in some Saos-2 cells, which reportedly used an alternative lengthening of telomeres (ALT) pathway to maintain telomere length rather than using telomerase (Bryan et al., 1997). A small percentage of Saos-2 cells also underwent apoptosis. Komata et al. (2001) recently showed that their hTERT/rev-caspase-6 construct could not kill two immortalized ALT pathway fibroblast cell lines. In comparison, osteosarcoma Saos-2 cells apparently have heterogeneous population of both ALT pathway cells and telomerase pathway cells. When Saos-2 cells were infected with a reporter construct Ad/hTERT-LacZ, and stained for β-gal-positive cells, about 25% of cells have weak hTERT promoter activity (data not shown).

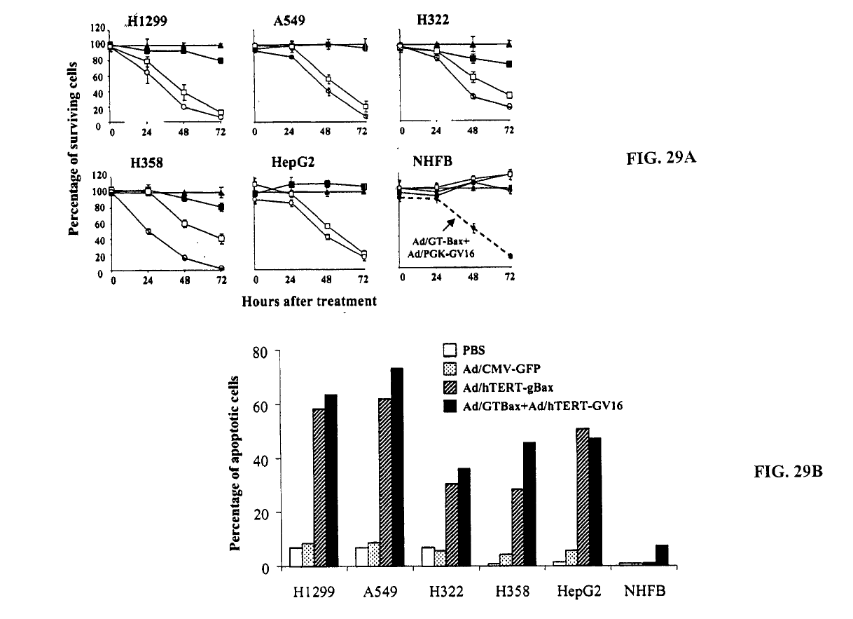

Ad/gBax induces apoptosis in human cancer cell in vitro. To further investigate the apoptosis-inducing effect of Ad/gBax as suggested by the above fluorescent microscopy observations, two additional experiments were performed. In the first experiment, cell viability was determined by an XTT assay at 24, 48 and 72 h after infection with Ad/gBax. Ad/CMV-GFP was used as a negative control, and Ad/GT-Bax +Ad/hTERT-GV16, a previously characterized binary system used to induce Bax expression, was used as a positive control. PBS was used as a mock infection. As shown in (FIG. 29A) both Ad/gBax and Ad/GT-Bax+Ad/TERT-GV16 resulted in comparable cell killing in all cancer cells, including cells of the four lungs cancer cell lines, and HepG2 liver cancer cells. In the NHFB cells, either treatment had an obvious effect on cell growth because of the low hTERT promoter activity in normal cells. It should be pointed out, however, that normal cells are susceptible to Bax-induced apoptosis, since these of a constitutively active promoter PGK to drive Bax expression in NHFB cells or normal human bronchial epithelial cells can kill these cells (Gu et al., 2000; also see FIG. 29A, NHFB panel, dashed line). Ad/CMV-GFP had no obvious effect on any of these cell lines. In the second experiment, FACS analysis was used to confirm that the growth suppression caused by GFP-Bax was due to apoptosis rather than to growth inhibition. All the cancer cells and NHFB cells were treated with Ad/gBax and binary vectors. Cells were harvested 72 h later and subjected to FACS analysis to determine the fraction of apoptotic cells, which was done by quantifying the sub-G1 population (FIG. 29B). Ad/gBax and Ad/GT-Bax+Ad/hTERT-GV16 produced comparable apoptosis populations in cancer cells but had minimal toxic effects on NHFB cells, suggesting that Ad/gBax can induce tumor-specific GFP-Bax expression and that GFP-Bax fusion protein is as potent as the Bax protein in inducing apoptosis in cancer cells. Similar results were obtained with other cancer cells. Expression of GFP, GFP-Bax and Bax in cancer cells was confirmed using H1299 cells as an example. Using either anti-GFP or anti-Bax antibodies detected a strong GFP-Bax fusion protein band. Both Ad/gBax and Ad/GT-Bax+Ad/hTERT-GV16 resulted in obvious caspase-3 activation and PARP cleavage, suggesting Bax induced apoptosis by activating the caspase-3 cascade. In contrast, 0.01 mg/ ml of Tc dramatically reduced the GFP-Bax level and caspase-3 activation, and the cytotoxic effect of Ad/gBax was also almost completely blocked (data not shown).

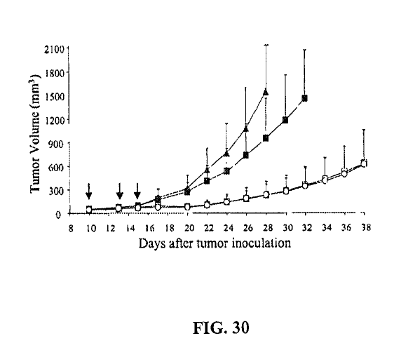

Ad/gBax induces tumor-specific GFP-Bax gene expression in vivo and suppresses xenograft tumor growth. To further evaluate the feasibility of using Ad/gBax for in vivo Bax gene therapy, H1299 tumors were established subcutaneously in nude mice and treated the tumors with Ad/gBax. Again, Ad/CMV-GFP was used as a negative control, Ad/GT-Bax+Ad/hTERT-GV16 as a positive control, and PBS as a mock infection. After three sequential intratumoral injections of the vectors, tumor size was monitored for 4 weeks. When tumors reached 15 mm in diameter, the mice had to be sacrificed according to institutional policy. Ad/gBax and Ad/GT-Bax+Ad/hTERT-GV16 produced the same level of tumor growth suppression, and this differed significantly from the results in tumors treated with PBS or Ad/CMV-GFP (FIG. 30, P≦0.01). It is noteworthy that tumors in Ad/gBax and Ad/GT-Bax+Ad/hTERT-GV16 treatment groups started to grow again after about 30 days. It has been previously shown that regrowing tumors after intralesional administration of adenovectors were susceptible to a second round of such treatment (Kagawa et al., 2001), suggesting that tumor regrowth may derive from initially untransduced tumor cells. The expression of GFP-Bax in tumors was confirmed by fluorescent microscopy and immunostaining. TUNEL staining of tumor sections confirmed that the apoptosis resulted from a single Ad/gBax intratumoral injection. In contrast, when Ad/CMV-GFP and Ad/gBax were systematically injected through the tail veins of mice, only the Ad/CMV-GFP induced strong fluorescence in the liver; the Ad/gBax did not induce detectable GFP-Bax expression. However, when a constitutively active promoter (PGK) was used to drive Bax expression, extensive expression of Bax, massive apoptosis of hepatocytes, and destruction of basic liver structure were observed. These results demonstrate that Ad/gBax can produce GFP-Bax and induce apoptosis in tumors and but not in normal tissue in vivo.

### EXAMPLE 8

### Combined TRAIL and Bax Gene Therapy Prolonged Survival in Mice with Ovarian Cancer Xenograft

Materials and Methods

Cell lines. The human ovarian cancer cells SKOV3ip and DOV13, and human lung cancer cell lines H1299 were grown in DMEM or RPMI 1640 medium supplemented with 10% heat-inactivated fetal bovine serum, antibiotics and glutamine. Normal human ovarian surface epithelial cells (Kruk et al., 1990) were grown in medium 199/MCDB-105, supplemented with 15% heat-inactivated fetal bovine serum, antibiotics, glutamine and epidermal growth factor (20 ng/ml).

Adenoviruses. The adenoviral vectors Ad/hTERT-LacZ, Ad/hTERT-GV16, Ad/CMV-LacZ, Ad/PGK-GV16, Ad/CMV-GFP, Ad/GT-Bax and Ad/gTRAIL were constructed as described previously.

In vitro vector administration. The optimal MOI was determined by infecting each cell line with Ad/CMV-LacZ and assessing the expression of β-galactosidase as described previously. MOIs that resulted in more than 80% of cells being stained blue were used in this experiment. These MOIs were 1000 particles for H1299, normal ovarian epithelial cells (NHOE); 2000 particles for DOV13 cell; and 6000 particles for the SKOV3ip cell.

Biochemical and flow cytometric assays. FACS analysis was performed as previously described to determine the level of in vitro GFP/TRAIL expression and number of apoptotic cells (Kagawa et al., 2000; Kagawa et al., 2001). In addition, Western blot analysis was performed as previously described using the following primary antibodies: anti-PARP (4C10-5; PharMingen, San Diego, Calif., USA), anti-Bax (6A7; PharMingen), anti-caspase-3 (CPP32; PharMingen); anti-caspase-8 (3-1-9; PharMingen); and anti-actin (AC-15; Sigma). Cell viability was determined via XTT assay as described in previous examples.

Mouse experiments and in vivo transgene expression assay. Animal experiments were conducted as previously described using SKOV3ip cells. Measurement of tumor volume, assessment of histopathological changes in the liver, spleen, intestine, lung, kidney, ovary, pancreas and heart; and serum liver enzyme ALT and AST assays were performed as described previously (Kagawa et al., 2000; Gu et al, 2002; Kagawa et al., 2001). In addition, toxicity after adenoviral vector delivery was monitored. After the mice were killed, various organs were harvested and fixed in 3.8% formalin in PBS. These organs were then sectioned, stained with hematoxylin and eosin, and examined histopathologically. For in vivo transgene expression assay, 26 days after cell inoculation, animals were given an intraperitoneal injection of Ad/hTERT-LacZ or a control vector at a dose of 1×10¹¹ vp/mouse. These animals were killed 2 days after treatment and their liver, spleen, intestine, kidney, pancreas, lung, and ovary were collected for frozen sectioning to test LacZ gene expression as described previously (Gu et al., 2000). Statistical analysis to assess the differences among the treatment groups was conducted as previously described.

Results

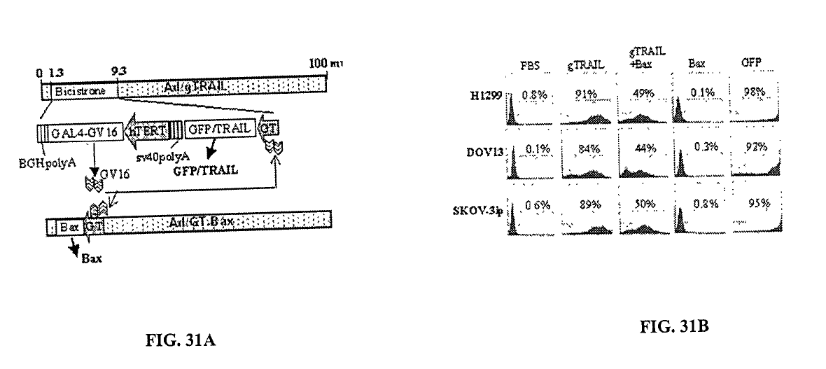

Transgene expression and apoptosis induction in cancer cells in vitro. The bicistronic adenoviral vector Ad/gTRAIL contains two expression cassettes. One cassette expresses the GFP/TRAIL fusion gene driven by the GAL4/TATA (GT) promoter, (Fang et al, 1997; Fang et al., 1998) while the other expresses the GAL4NVP16 (GV16) fusion gene driven by the hTERT promoter. As previously shown, the GT promoter is silent in mammalian cells in the absence of GV16 but highly active in the presence of GV16 (Fang et al., 1998). Thus, expression of GAL4/GV16 driven by the hTERT promoter leads to activation of the GT promoter and expression of GFP/TRAIL fusion protein. As also previously shown, the adenoviral vector Ad/GT-Bax expresses the Bax gene when it is co-administered with an adenoviral vector expressing the GV16 fusion protein (Gu et al., 2000; Kagawa et al., 2000). Therefore, it is believed that Ad/gTRAIL expresses the GFP/TRAIL fusion protein in hTERT-active cells. When co-infected with Ad/GT-Bax, Ad/gTRAIL also induces Bax gene expression in hTERTactive cells (FIG. 31). Thus, unless otherwise stated, all the studies undertook used Ad/gTRAIL for the expression of GFP/TRAIL; Ad/hTERT-GV16 plus Ad/GT-Bax for the expression of Bax; Ad/gTRAIL plus Ad/GT-Bax for the expression of both GFP/TRAIL and Bax; phosphate-buffered saline (PBS) and Ad/CMV-GFP plus Ad/GT-Bax were used as mock or vector controls.

To determine the level of the transgene expression in cancer cells, cells were harvested 48-72 h after treatment, and the expression of GFP/TRAIL and Bax was analyzed using FACS and Western blot analysis, respectively. Cells treated by PBS or Ad/CMV-GFP±Ad/GT-Bax were used as mock or vector control. Cells were harvested 48-72 h after the treatment and the expression of GFP/TRAIL and Bax were analyzed by FACS and Western blot analysis, respectively. Treatment using Ad/gTRAIL alone resulted in GFP/TRAIL expression in 84% to 91% of the cancer cells tested (FIG. 31B). However, treatment using Ad/gTRAIL plus Ad/GT-Bax, resulted in GFP/TRAIL expression in 43% to 56% of the cells, reflecting an Ad/gTRAIL dose reduction in this group. GFP-positive cells were not detected when cells were treated using PBS or Ad/hTERT-GV16 plus Ad/GT-Bax. Additionally over-expression of the Bax protein was observed only in cells treated using Ad/gTRAIL plus Ad/GT-Bax or Ad/hTERT-GV16 plus Ad/GT-Bax. These results demonstrated that Ad/gTRAIL can express GFP/TRAIL fusion protein itself and induce the Bax gene expression when co-administered with Ad/GT-Bax.

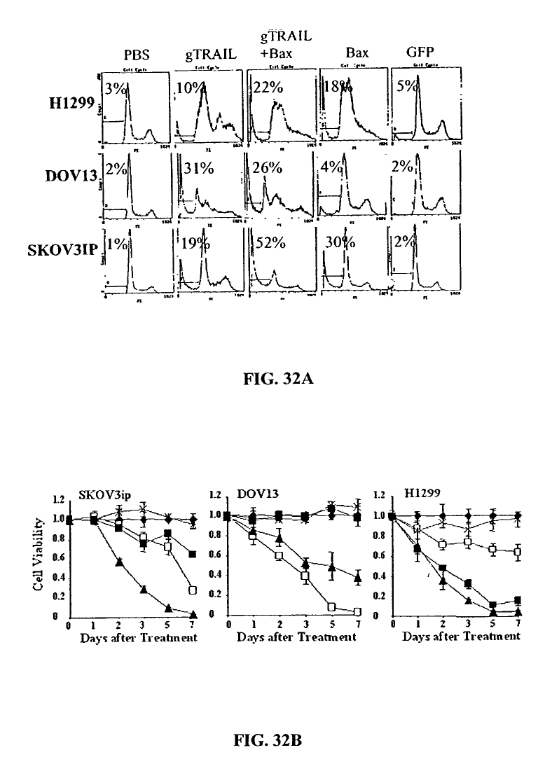

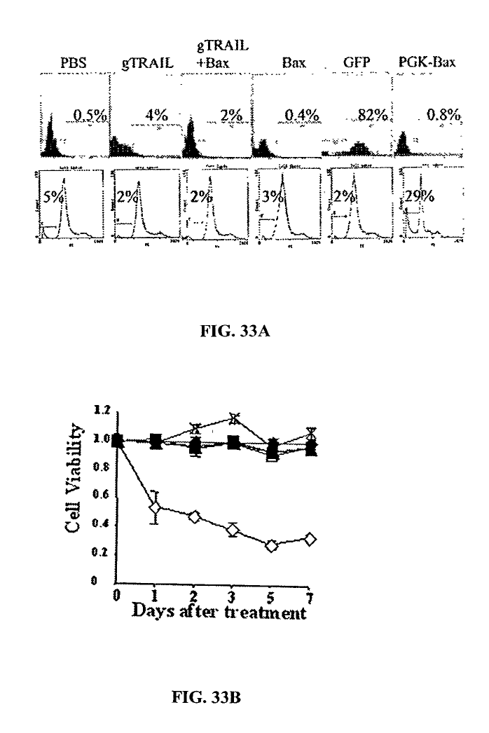

As previously reported, the direct transferal of the Bax gene or GFP/TRAIL fusion gene will induce apoptosis of cancer cells (Kagawa et al., 2000; Kagawa et al., 2001). To examine apoptosis induction by the GFP/TRAIL and Bax gene both separately and together, the activation of caspase-8 and -3, and cleavage of poly (ADP-ribose) polymerase (PARP) were tested using the same SKOV3ip samples that were used in testing for Bax expression. Activation of caspase-8 and -3 and cleavage of PARP was detected in cells treated using vectors expressing the GFP/TRAIL and/or Bax genes but not in cells treated using PBS or control vectors. In addition, to quantify the level of apoptosis induction in cancer cells, the DNA contents in the human ovarian cancer cell lines SKOV3ip and DOV13, and human lung cancer cell line H1299 were analyzed using FACS. Apoptosis was induced in SKOV3ip and H1299 cells when treated using GFP/TRAIL- or Bax- expressing vectors alone. Nevertheless, even though the same total vector dose was used in each cell line, apoptosis induction was more profound in cells treated using Ad/gTRAIL plus Ad/GT-Bax, which were the vectors expressing both GFP/TRAIL and Bax. This phenomenon was more prominent in SKOV3ip cells (FIG. 32A). However, in DOV13 cells, treatment using Ad/gTRAIL alone resulted in more apoptotic cells than did that using Ad/gTRAIL plus Ad/GT-Bax. This can be explained by the fact that the DOV13 cells were sensitive to the GFP/TRAIL-expressing vector, but highly resistant to the Bax-expressing vector. Thus, at the same total multiplicity of infection (MOI), the dose of the effective vector (Ad/gTRAIL) was doubled in the group that received Ad/gTRAIL alone when compared with the group that received Ad/gTRAIL plus Ad/GT-Bax. Nevertheless, treatment of DOV13 cells using Ad/gTRAIL plus Ad/GT-Bax effectively elicited apoptosis of DOV13 cells, while treatment using Ad/hTERT-GV16 plus Ad/GT- Bax did not.

To further document the cell-killing effects of the Bax and GFP/TRAIL genes both separately and together, an XTT assay was performed to measure cell viability within 1 week after treatment (FIG. 32B). The results of this assay were inconsistent with those of FACS analysis. Specifically, the DOV13 cells were highly sensitive to the GFP/TRAIL-expressing vector, but highly resistant to the Bax-expressing vector. The cell-killing effect of the combined therapy was therefore derived mainly from the expression of GFP/TRAIL. Furthermore, because the dose of effective vector (Ad/gTRAIL) in the combined group was only half of that in the group that received Ad/gTRAIL alone group, the cell-killing effect was reduced when compared with that in cells treated using Ad/gTRAIL alone. In contrast, in SKOV3ip and H1299 cells, treatment using the GFP/TRAIL- or Bax-expressing vectors alone elicited substantial cell death. Combined therapy using the GFP/TRAIL and Bax genes enhanced cell killing in these two cell lines. This enhancement of cell killing is more profound in SKOV3ip cells that are modestly sensitive to both the Bax- and the GFP/TRAIL-expressing vectors.

Transgene expression and apoptosis induction in normal human ovarian surface epithelial cells in vitro. To test whether treatment using Ad/gTRAIL plus Ad/GT-Bax resulted in transgene expression and apoptosis induction in normal cells, FACS analysis and the XTT assay were performed using normal human ovarian surface epithelial cells (NHOE) after treatment using various vectors. Also, Ad/PGK-GV16 plus Ad/GT-Bax was used as a positive control for apoptosis induction in those cells because as previously found, most normal cells are sensitive to Bax gene overexpression (Gu et al., 2000; Kawaga et al., 2000). FACS analysis showed that less than 5% of the cells were GFP-positive after treatment using Ad/gTRAIL at an MOI of 1000 vp/cells (FIG. 33A). In comparison, more than 80% of the cells were GFP-positive when treated using Ad/CMV-GFP plus Ad/GT-Bax at the same MOI (ratio, 1:1). This result suggests that normal ovarian epithelial cells are sensitive to adenoviral infection but have low hTERT promoter activity, which is consistent with previous observation that the hTERT promoter is highly active in cancer cells but relatively quiescent in normal cells in vitro (Gu et al., 2000; Gu et al., 2002). FACS analysis and XTT assay showed that treatment using Ad/gTRAIL, Ad/gTRAIL plus Ad/GT-Bax, and Ad/hTERT-GV16 plus Ad/GT-Bax all resulted in only a background level of cell killing in normal ovarian epithelial cells (FIGS. 33A & 33B). In comparison, substantial apoptosis was induced when the cells were treated using Ad/PGK-GV16 plus Ad/GT-Bax, suggesting that the normal ovarian epithelial cells are sensitive to Bax overexpression. These results were also consistent with previous observation that normal cells are susceptible to Bax gene overexpression and that the hTERT promoter can be used to prevent transgene expression and its related toxicity in normal cells (Gu et al., 2000).

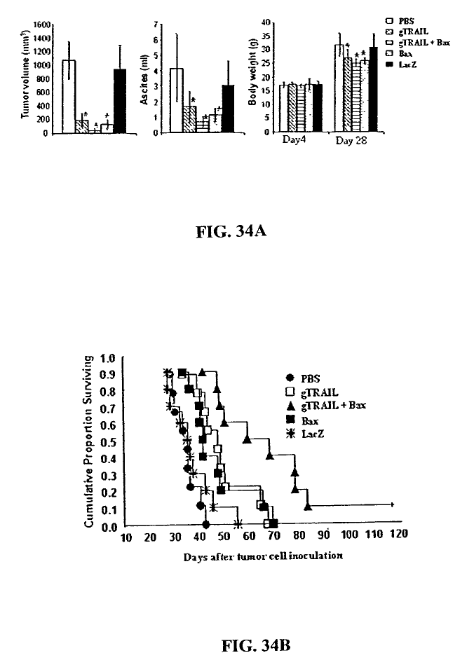

Suppression of intraperitoneal tumor growth and ascite formation in vivo. The effect of combined therapy in vivo in an abdominally spread tumor model derived from SKOV3ip cells was also tested. Specifically, 1×10⁶ SKOV3ip cells were inoculated into nude mice intraperitoneally. Four days after cell inoculation, the mice were grouped randomly (15 mice per group), and intraperitoneal administration of vectors at a total dose of 6×10⁶ viral particles (vp)/mouse/treatment was initiated. When two vectors were used, the total dose remained the same while the ratio of the two vectors was set at 1:1. Animals that received PBS were used as mock-treatment controls. At weeks after cell inoculation, five mice in each group were killed, and the volume of their ascites and largest tumor nodule in their abdominal cavity were measured. In addition, the mice body weight was determined before treatment was started and 4 weeks after tumor-cell inoculation. No differences in the volume of ascites, volume of the largest tumor nodule in the abdominal cavity, or body weight were found in the animals that received PBS or control vector (FIGS. 34A & 34B). However, when compared with the pretreatment amount, the mice's body weight was higher in all of the groups 4 weeks after tumor-cell inoculation. This increase in body weight may have reflected the formation of ascites in these animals, also the increase was less prominent in animals that received Ad/gTRAIL, Ad/gTRAIL plus Ad/GT-Bax or Ad/hTERT-GV16 plus Ad/GT-Bax. In these three groups, the volume of both ascites and largest tumor nodules in the abdominal cavity was significantly reduced when compared with that in the animals that received PBS or control vector (P<0.05), suggesting that antitumor activities were elicited by these treatments. Additionally, the volume of the largest tumor nodules in animals that received Ad/gTRAIL plus Ad/GT-Bax was significantly lower than that in animals that received Ad/gTRAIL or Ad/hTERT-GV16 plus Ad/GT-Bax (P<0.05). However, the number of the tumor nodules in the abdominal cavity was not countable in all of the groups. There were no tumor-free mice in any of the groups when the mice were killed.

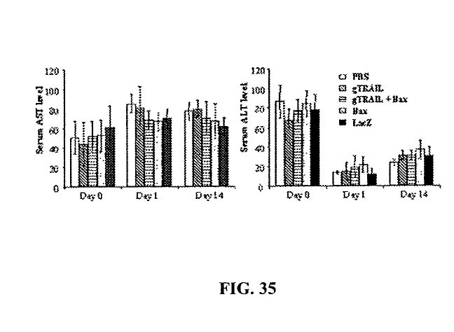

The remaining 10 mice in each group were monitored for survival. The results showed that treatments using Ad/gTRAIL or Ad/hTERT-GV16 plus Ad/GT-Bax significantly improved the survival duration in animals that received it when compared with that in animals that received PBS or the control vector (P<0.05). Furthermore, combined TRAIL and Bax treatment prolonged survival significantly when compared with treatment using either gene alone (P<0.05) (FIG. 35). The mean survival durations in mice that received PBS, the control vector, the TRAIL gene, the Bax gene, and both the TRAIL and Bax genes was 35, 37, 49, 48 and 68 days, respectively. Thus, significantly prolonged survival can be achieved using combined TRAIL and Bax gene therapy without an increase in vector doses.

Toxicity and transgene expression after intraperitoneal vector administration. As previously shown, the hTERT promoter is silent in murine liver, but active in murine tumor cells (Gu et al., 2000; Gu et al., 2002). Transgene expression in murine liver was not detected after systemic administration of an adenovirus expressing a transgene from the hTERT promoter in that study. However, the level of transgene expression after intraperitoneal vector administration remained unknown. Therefore, to test transgene expression from the hTERT promoter after intraperitoneal administration in the present study, mice bearing abdominal tumors derived from SKOV3ip cells were given an intraperitoneal injection of Ad/hTERT-LacZ or a control vector at a single dose of 1×10¹¹ vp/mouse. The animals were killed 2 days after injection and collected their liver, spleen, intestine, kidney, pancreas, lung, ovary and peritoneum for frozen sectioning to check the level of in vivo gene expression using X-gal staining. Expression of bacterial β- galactosidase was detected only in abdominal tumors, not in the collected organs or in the serosa or peritoneal cavity. In comparison, bacterial β-galactosidase expression was not detected in normal tissue, as well as in tumors after treatment using a control vector. These results confirmed the targeted gene expression using the hTERT promoter after intraperitoneal vector administration. The toxicity of both intraperitoneal administration of the GFP/TRAIL- and Bax-expressing vectors and the combined therapy regimen described above was also tested. For this testing, serum samples were collected before treatment started and both 2 and 14 days after treatment ended. Analysis of the serum alanine transaminase (ALT) and aspartate transaminase (AST) level showed that they were both in the normal range in all of the treatment groups and at all of the time-points tested (FIG. 35). Histopathological changes in liver, spleen, intestine, lung, kidney, ovary, pancreas and heart were also examined after these organs were collected. No obvious lesions were found in any of the organs in either treatment group. Taken together, these results suggest that intraperitoneal administration of the GFP/TRAIL and Bax genes separately or combined resulted in minimal toxicity when transgene expressions was controlled by the hTERT promoter.

### EXAMPLE 9

### Combined Ad/gTRAIL and Chemotherapy for Treatment of Cancers

Materials and Methods

Tumor Model. Human breast cancer lung metastatic tumor model were established in 6 to 8 weeks old nude mice by injecting of 2×10⁶ 231/ADR breast cancer cells/0.2 ml into the tail vein of each mouse. Then animals were randomized into different treatment group. There were five groups including Ad/CMV-GFP, Paclitaxol, Ad/gTRAIL, Paclitaxol+Ad/CMV-GFP, and Paclitaxol+Ad/gTRAIL. Treatment started one week later after inoculating the cells into mice. Paclitaxol was administered intravenously via the tail vein on day 1 and 21 (4 mg/kg). Adenovirus (Ad/GFP or Ad/gTRAIL) was delivered by aerosol on day 1, 7, 14, 21, and 28. The mouse was sacrificed on day 35 after starting treatment. Lungs were harvested for either histopathological assay or for surface tumor nodule assay (by injecting inks to lung).

The aerosol vector administration are the following: adenovirus of 1×10¹² particles was diluted in PBS to a final volume of Iml and was mixed with 500 μl (5 mg) of protamine. The Protamine-adenovirus complex was mixed with 5 ml hydrocortisone (concentration of 250 ng/ml in H2O) after incubating 10 min at room temperature. The complex was placed into the nebulizer chamber. The aerosol from the nebulizer was passed through a sealed plastic cage that housed the mice. The exposure required approximately 50 minutes, during which time the mice were allowed to move freely about the cage.

Results

The breast cancer cell line MDA-MB-231 repeatedly treated with doxorubicin resulted in selection of doxorubicin-resistant cells (named 231/ADR). Whether the combination of Ad/gTRAIL adenovector with chemotherapeutic agents can be used to treat these TRAIL-resistant or chemo-resistant cancer cells was tested.

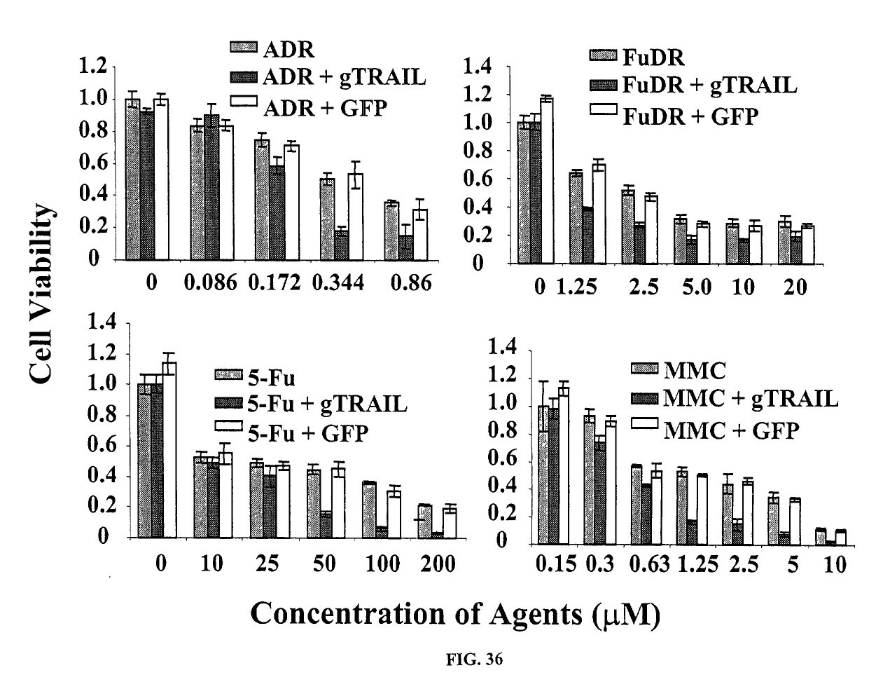

FIG. 36 showed that human colon cancer cell lines DLD1/TRAIL-R, a cell line resistant to Ad/gTRAIL can be sensitized to Ad/gTRAIL by chemotherapeutic agents, such as doxorubicin (ADR), floxuridine (FuDR); fluorouracil (5-FU) and mutamycin (MMC). In this case, DLD1/TRAIL-R cells were treated with PBS or Ad/gTRAIL or Ad/CMV-GFP at 1000 viral particles/cell. Chemotherapeutic agents concentrations are shown on the bottom of each graph. The concentration 0 indicates that cells treated with PBS, Ad/gTRAIL or Ad/CMV-GFP only. Cell viability was determined at 96 h after treatment. Levels are mean+/−SD of two quadruplet assays. As shown in the FIG. 36, doxorubicin, fluorouracil and mutamycin all can sensitize DLD1/TRAIL-R to Ad/gTRAIL at certain concentrations. Western blot analysis of DLD1/TRAIL-R cells after combination treatment showed an increase in PARP cleavage. Cells were treated with adenovector at 1000 moi (vp), 5-FU 50 μm and 1.25 μm MMC. In combination group, the dose were the same as the single agent group. Cells were harvested at 96 h after treatment and cell lysate was used for Western blot analysis.

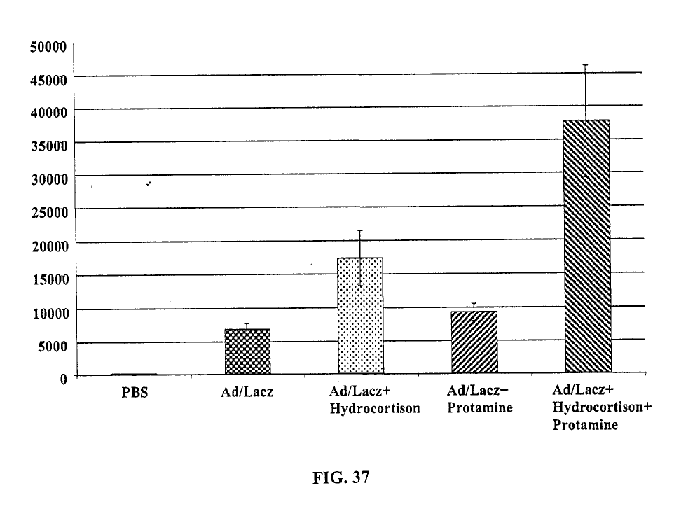

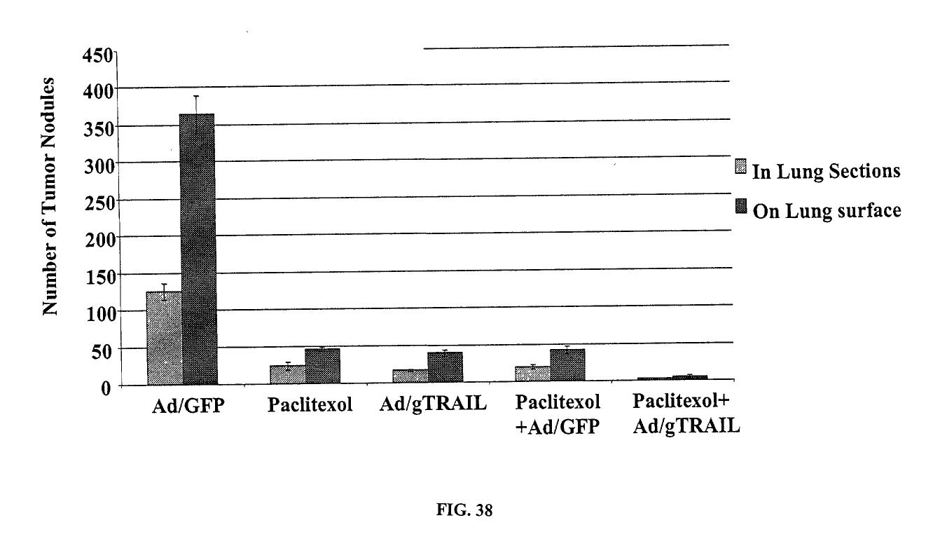

Treatment of lung metastasis by aerosolized vector administration was also tested. The results demonstrated that transgene expression in lung can be dramatically augmented by including protamine and hydrocortisone in the vector solution. FIG. 37 show that reporter gene LacZ expression is dramatically increased by including protamine (1 mg/ml) and hydrocortisone (250 ng/ml). When protamine and hydrocortisone are both present in the solution, the expression level is increase further. Subsequent aerosol administration of adenovectors will contain protamine and hydrocortisone in the vector solution.

FiIG. 38 show the results of aerosolized vector administration of Ad/gTRAIL in combination with paclitaxol for treatment of lung metastasis derived from breast cancer cells 231/ADR that are resistant to doxorubicin. The results showed that animals treated with Ad/CMV-GFP developed numerous tumor nodules in the lung. In comparison, treatment with either paclitaxol and Ad/gTRAIL dramatically reduced the numbers of tumor nodules. Combination of Ad/gTRAIL with paclitaxol further reduced the tumor numbers (hard to find any). However, combination of Ad/CMV-GFP with paclitaxol has the similar results as paclitaxol alone.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

VII. References

The following references, to the extent that they provide exemplary procedural or other details supplementary to those set forth herein, are specifically incorporated herein by reference.

U.S. Pat. No. 4,797,368

U.S. Pat. No. 5,139,941

U.S. Pat. No. 5,359,046

U.S. Pat. No. 5,464,758

U.S. Pat. No. 5,925,565

U.S. Pat. No. 5,935,819

U.S. Pat. No. 5,994,136

U.S. Pat. No. 6,013,516

Andreeffet al., Leukemia, 13:1981-1892, 1999.

Andriani et al., JNCI, 93(17):1314-24, 2001.

Ausubel et al., In: Current Protocols in Molecular Biology, John, Wiley and Sons, Inc, NY, 1994.

Baichwal and Sugden, In: Gene Transfer, Kucherlapati (ed.), NY, Plenum Press, 117-148, 1986.

Bakhshi et al., Cell, 41(3):899-906, 1985.

Bedzyk et al., J. Biol. Chem., 265(30):18615-18620, 1990.

Blomer et al., J. Virology, 71(9):6641-6649, 1997.

Bodnar et al., Science, 279:349-352, 1998.

Burbage et al., Leuk. Res., 21(7):681-690, 1997.

Caldas et al., Nat. Genet., 8(1):27-32, 1994.

Carbonelli et al., FEMS Microbiol. Lett., 177(1):75-82, 1999.

Chandler et al., Proc. Natl. Acad. Sci. USA, 94(8):3596-601, 1997.

Chaudhary et al., Proc. Natl. Acad. Sci. USA, 87(3):1066-1070, 1990.

Chen et al., J. Clin. Invest., 96:2775-2782, 1995.

Cheng et al., Cancer Res., 54(21):5547-5551, 1994.

Cleary and Sklar, Proc. Natl. Acad. Sci. USA, (21):7439-7443, 1985.

Cleary et al., J. Exp. Med., 164(1):315-320, 1986.

Cocea, Biotechniques, 23(5):814-816, 1997.

Cong et al., Hum. Mol. Genet., 8:137-142, 1999.

Cotten et al., Proc. Natl. Acad. Sci. USA, 89(13):6094-6098, 1992.

Counter et al., Oncogene, 16:1217-1222, 1998.

Coupar et al., Gene, 68:1-10, 1988.

Courey and Tjian, Cell, 55:887-898, 1988.

Culver et al., Science, 256(5063):1550-1552, 1992.

Curiel, Nat. Immun., 13(2-3):141-64, 1994.

Fang et al., J. Biol. Chem., 273:4972-4975, 1998.

Fang et al., J. Virology, 71:4798-4803, 1997.

Fang et al., Gene Ther., 1:247-254, 1994.

Fang et al., Hum. Gene Ther., 6:1039-1044, 1995.

Fang et al., Anal. Biochem., 254:139-143, 1997.

Fang et al., Gene Ther., 3:217-222, 1996.

Feldman et al., Stem Cells, 15:386-395, 1997.

Forster and Symons, Cell, 49(2):211-220, 1987.

Friedmann, Science, 244:1275-1281, 1989.

Gerlach et al., Nature (London), 328:802-805, 1987.

Giniger and Ptashne, Nature, 330:670-672, 1987.

Greenberg et al., Mol Endo, 8:230-9, 1994.

Greenberg et al., Proc Natl. Acad. Sci. USA, 92:3439-43, 1995.

Grunhaus et al., Seminar in Virology, 200(2):535-546, 1992.

Gu et al., Cancer Res,. 60:5359-5364, 2000.

Gu et al., Gene Therapy, 9:30-37, 2002.

Hahn et al., Nature, 400:464-468, 1999.

Harrington et al., Gene Dev., 11:3109-3115, 1997.

Hauck and Stanners, J. Biol. Chem., 270:3602-3610, 1995.

Hillen and Wissmann, In: Protein-Nucleic Acid Interaction, Saeger and Heinemann (Eds.) Macmillan, London, 10: 143-162, 1989.

Hinoda et al., Japanese J. Clin. Oncol., 21:75-81, 1991.

Hollstein et al., Science, 253(5015):49-53, 1991.

Horikawa et al., Cancer Res., 59:826-830, 1999.

Horwich et al., Virol., 64:642-650, 1990.

Howard et al., Leukemia, 13:1608-1616, 1999.

Hsu et al., Devel. Biol., 21:154-160, 1985.

Hussussian et al., Nat. Genet., 8(1):15-21, 1994.

Inouye and Inouye, Nucleic Acids Res., 13: 3101-3109, 1985.

Jessup and Thomas, Cancer and Metastasis Rev,. 8:263-280, 1989.

Ji et al., Gene Ther., 6:393-402, 1999.

Joyce, Nature, 338:217-244, 1989.

Kagawa et al., Cancer Res., 3330-3338, 2001.

Kagawa et al., Cancer Res., 60:1157-1161, 2000.

Kagawa et al., Gene Therapy, 7:75-79, 2000.

Kamb et al., Nat. Genet., 8(1):23-2, 1994.

Kelleher and Vos, Biotechniques, 17(6):1110-7, 1994.

Kerr et al., Br. J Cancer, 26(4):239-257, 1972.

Kijima et al., Cancer Res., 59:4906-4911, 1999.

Kilian et al., Hum. Mol. Genet., 12:2011-2019, 1997.

Kim and Chech, Proc. Natl. Acad. Sci. USA, 84(24):8788-8792, 1987.

Koch et al., Gene Ther., 3:278-283, 2001.

Koch et al., Mol. Ther., 3:278-283, 2001.

Kruk et al., Lab. Invest., 63:132-136, 1990.

Latham et al., Cancer Res., 60(2):334-41, 2000.

Laughlin et al., J. Virology, 60(2):515-24, 1986.

Lebkowski et al., Mol. Cell. Biol., 8(10):3988-96, 1988.

Levenson, Transue, Roninson, Hum. Gene Ther., 9(8): 1233-6, 1998.

Lidor et al., Am. J. Obstet. Gynecol., 177(3):579-585, 1997.

Lu et al., Cancer Res., 59:5202-5208, 1999.

Macejak and Samow, Nature, 353:90-94, 1991.

Mann, Mulligan, Baltimore, Cell, 33:153-159, 1983.

Massuda, et al., Proc. Natl. Acad. Sci. USA, 94(26):14701-14706, 1997.

McLaughlin et al., J Virology, 62(6):1963-73, 1988.

Mermod et al., Cell, 58:741-753, 1989.

Meyerson et al., Cell, 90:785-795, 1997.

Michel and Westhof, J. Mol. Biol., 216:585-610, 1990.

Miller et al., Am. J. Clin. Oncol., 15(3):216-21, 1992.

Muzyczka, Curr. Top Microbiol. Immunol., 158:97-129, 1992.

Nakamura et al., Science, 277:955-959, 1997.

Nakayama et al., Nature Genet., 18:65-68, 1998.

Naldini et al., Science, 272(5259):263-267, 1996.

Neumaier et al., J. Biol. Chem., 263:3202-3207, 1988.

Nicolas and Rubinstein, In: Vectors: A survey of molecular cloning vectors and their uses, Rodriguez and Denhardt (Eds), Stoneham: Butterworth, 494-513, 1988.

Nobori et al., Nature, 368(6473):753-756, 1994.

Oikawa et al., Biochem. Biophys. Res. Comm., 146:464-469, 1987.

Okamoto et al., Proc. Natl. Acad. Sci. USA, 1(23): 11045-1104994.

Oreffo et al., Mol. Carcinog., 21:37-49, 1998.

Orlow et al., Int. J Oncol., 15(1):17-24, 1999.

Osaki et al., Cancer Res., 54:5258-5261, 1994.

Paskind et al., Virology, 67:242-248, 1975.

Paxton et al., Proc. Natl. Acad. Sci. USA, 84:920-924, 1987.

Pelletier and Sonenberg, Nature, 357:173-176, 1992.

Remington's Pharmaceutical Sciences, 18th Edition, Chapter 61, pages 1289-1329, 1990.

Rennie et al., Mol Endo, 7:23-36, 1993.

Richards et al., Hum. Gene Ther., 6:881-893, 1995.

Ridgeway, In: Vectors: A survey of molecular cloning vectors and their uses. Rodriguez and Denhardt (Eds), Stoneham: Butterworth, 467-492, 1988.

Robbins et al., Int. J Cancer, 53:892-897, 1993.

Roux et al., Proc. Natl. Acad. Sci. USA, 86:9079-9083, 1989.

Sarver et al., Science, 247:1222-1225, 1990.

Scanlon et al., Proc. Natl. Acad. Sci. USA, 88:10591-10595, 1991.

Schrewe et al., Mol. Cell. Biol., 10:2738-2748, 1990.

Schuur et al., J. Biol. Chem., 271(12):7043-7051, 1996.

Serrano et al., Science, 267(5195):249-252, 1995.

Serrano et al., Nature, 366:704-707, 1993.

Strom et al., J. Nat. Cancer Inst., 68:771-778, 1982.

Takakura et al., Cancer Res., 59:551-557, 1999.

Tanaka and Herr, Cell, 60:375-386, 1990.

Thompson et al., J. Clin. Lab. Anal., 5:344-366, 1991.

Tratschin et al., Mol. Cell. Biol., 4:2072-2081, 1984.

Triezenberg et al., Genes Dev. 2(6):718-729, 1988.

Tsujimoto and Croce, Proc. Natl. Acad. Sci. USA, 83(14):5214-5218, 1986.

Tsujimoto et al., Science, 228(4706):1440-1443, 1985.

Watanabe et al., Blood, 87:5032-5039, 1996.

Weinberg, Science, 254(5035):1138-1146, 1991.

Weinrich et al., Nature Genet., 17:498-502, 1997.

Witzgall et al., Proc. Natl. Acad. Sci. USA, 91:4514-4518, 1994.

Yang et al., Proc. Natl. Acad. Sci. USA, 91:4407-4411, 1994.

Zufferey et al., Nat. Biotechnol., 15(9):871-875, 1997.

What is claimed is:

1. A method for expressing gene product in a cell type-preferential manner comprising:
(a) providing a first expression cassette comprising a cell type-preferential promoter that directs the expression of a nucleic acid encoding a transcription factor;
(b) providing a second expression cassette comprising an inducible promoter, responsive to said transcription factor, that directs the expression of a nucleic acid encoding a selected polypeptide; and
(c) transferring said first and second expression cassettes into a cell in which said cell type-specific preferential promoter is active,
wherein said transcription factor is expressed and directs expression of said selected polypeptide.

2. The method of claim 1, wherein said cell type-preferential promoter is hTERT, CEA, PSA, probasin, ARR2PB or AFP.

3. The method of claim 1, wherein said transcription factor is GAL4-VP16 fusion and said inducible promoter is GAL4/TATA.

4. The method of claim 1, wherein said transcription factor is tetR-VP16 fusion and said inducible promoter is tet operator.

5. The method of claim 1, wherein said first and second expression cassettes are located in different expression constructs.

6. The method of claim 1, wherein said first and second expression cassettes are located in the same expression construct.

7. The method of claim 1, wherein at least one of said expression cassettes is located in a viral expression construct.

8. The method of claim 1, wherein at least one of said expression cassettes is located in a non-viral expression construct.

9. The method of claim 1, wherein said cell is a tumor cell.

10. The method of claim 1, wherein said selected polypeptide is a tumor suppressor, an inducer of apoptosis, a cytokine, an enzyme or a toxin.

11. The method of claim 1, wherein said tumor suppressor is p53, Rb, PTEN, BRCA1 and BRCA2.

12. The method of claim 1, wherein said inducer of apoptosis is Bax, Bad, Bid, Bik, Bak, TRAIL, FasL, Noxa, PUMA, p53AIP1, TGF-β, Granzyme A or Granzyme B.

13. The method of claim 10, wherein said cytokine is IL-2, IL-4, IL-10, IL-12, GM-CSF, MCP-3, TNF-α, or INF-β.

14. The method of claim 10, wherein said enzyme is cytosine deaminase.

15. The method of claim 10, wherein said toxin in ricin A chain, cholera toxin and pertussis toxin.

16. The method of claim 9, wherein said tumor cell is selected from the group consisting of a brain tumor cell, a head & neck tumor cell, an esophageal tumor cell, a lung tumor cell, a thyroid tumor cell, a stomach tumor cell, a colon tumor cell, a liver tumor cell, a kidney tumor cell, a prostate tumor cell, a breast tumor cell, a cervical tumor cell, an ovarian tumor cell, a testicular tumor cell, a rectal tumor cell, a skin tumor cell or a blood tumor cell.

17. A method of treating a human subject having cancer comprising:
(a) providing a first expression cassette comprising a CEA or hTERT promoter that directs the expression of a nucleic acid encoding a transcription factor;
(b) providing a second expression cassette comprising an inducible promoter, responsive to said transcription factor, that directs the expression of a nucleic acid encoding a therapeutic polypeptide; and
(c) transferring said first and second expression constructs into a cancer cell in said subject,
wherein said transcription factor is expressed and directs expression of said therapeutic polypeptide.

18. The method of claim 17, wherein (i) said transcription factor is GAL4-VP16 fusion and said inducible promoter is GAL4/TATA, or (ii) said transcription factor is tetR-VP 16 fusion and said inducible promoter is tet operator.

19. The method of claim 17, wherein said first and second expression cassettes are located in different expression constructs.

20. The method of claim 17, wherein said first and second expression cassettes are located in the same expression construct.

21. The method of claim 17, wherein at least one of said expression cassettes is located in a viral expression construct.

22. The method of claim 17, wherein at least one of said expression cassettes is located in a non-viral expression construct.

23. The method of claim 21, wherein said viral expression construct is an adenoviral expression construct, a herpesviral expression construct, a retroviral expression construct, a vaccinia viral expression construct, an adeno-associated viral expression construct or a polyoma viral expression construct.

24. The method of claim 1, wherein said selected polypeptide is a tumor suppressor, an inducer of apoptosis, a cytokine, an enzyme or a toxin.

25. The method of claim 24, wherein said tumor suppressor is p53, Rb, PTEN, BRCA1 and BRCA2.

26. The method of claim 24, wherein said inducer of apoptosis is Bax, Bad, Bid, Bik, Bak, TRAIL, FasL, Noxa, PUMA, p53AIP1, TGF-β, Granzyme A or Granzyme B.

27. The method of claim 24, wherein said cytokine is IL-2, IL-4, IL-10, IL-12, GM-CSF, MCP-3, TNF-β or INF-β.

28. The method of claim 24, wherein said enzyme is cytosine deaminase.

29. The method of claim 24, wherein said toxin in ricin A chain, cholera toxin and pertussis toxin.

30. The method of claim 17, wherein said cancer is selected from the group consisting of brain cancer, head & neck cancer, esophageal cancer, lung cancer, thyroid cancer, stomach cancer, colon cancer, liver cancer, kidney cancer, prostate cancer, breast cancer, cervical cancer, ovarian cancer, testicular cancer, rectal cancer, skin cancer or blood cancer.

31. The method of claim 17, further comprising administering a second cancer therapy comprising surgery, immunotherapy, chemotherapy or radiation therapy.

32. A method for treating a human cancer patient comprising:
(a) providing a non-viral expression cassette comprising an hTERT promoter that directs the expression of a nucleic acid encoding a tumor suppressor or an inducer of apoptosis; and
(b) administering said expression cassette into said subject,
wherein said tumor suppressor or inducer of apoptosis is expressed and inhibits growth of cancer cells, thereby treating said cancer.

33. The method of claim 32, wherein said tumor suppressor is p53, Rb, PTEN, BRCA1 and BRCA2.

34. The method of claim 32, wherein said inducer of apoptosis is Bax, Bad, Bid, Bik, Bak, TRAIL, FasL, Noxa, PUMA, p53AIP1, TGF-β, Granzyme A or Granzyme B.

35. The method of claim 32, wherein said cancer cells are killed.

36. The method of claim 32, wherein at least one of said expression cassettes is located in a viral expression construct.

37. The method of claim 36, wherein said nucleic acid encoding a tumor suppressor or an inducer of apoptosis further encodes a screenable marker fused to said tumor suppressor or said inducer of apoptosis.

38. The method of claim 32, wherein at least one of said expression cassettes is located in a non-viral expression construct.

39. The method of claim 32, further comprising administering a second cancer therapy comprising surgery, immunotherapy, chemotherapy or radiation therapy.

40. The method of claim 32, wherein said cancer is selected from the group consisting of brain cancer, head & neck cancer, esophageal cancer, lung cancer, thyroid cancer, stomach cancer, colon cancer, liver cancer, kidney cancer, prostate cancer, breast cancer, cervical cancer, ovarian cancer, testicular cancer, rectal cancer, skin cancer or blood cancer.


SEQUENCE LISTING (2 sequences)

SEQ ID NO: 1 - DNA, 378 bp, Artificial Sequence
  Description of Artificial Sequence  Synthetic Primer
caccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgc

SEQ ID NO: 2 - DNA, 425 bp, Artificial Sequence
  Description of Artificial Sequence  Synthetic Primer
gccctggagagcatggggagacccgggacctgctgggtttctctgtcacaaaggaaaataatccccctggtgtgacagacccaaggacagaacacagcagaggtcagcactggggaaagacaggttgtccacaggggatgggggtccatccaccttgccgaaaagatttgtctgaggaactgaaaatagaagggaaaaaagaggagggacaaaagaggcagaaatgagaggggaggggacagaggacacctgaataaagaccacacccatgacccacgtgatgctgagaagtactcctgccctaggaagagactcagggcagagggaggaaggacagcagaccagacagtcacagcagccttgacaaaacgttcctggaactcaagctcttctccacagaggaggacagagcagacagcagagac